(19) United States
(12) Patent Application Publication
(10) Pub. No.: US 2022/0281863 A1
(43) Pub. Date: Sep. 8, 2022

(54) COMPOUNDS, COMPOSITIONS AND METHODS OF TREATING DISORDERS

(71) Applicant: Reverie Labs, Inc., Cambridge, MA (US)

(72) Inventors: Mark Rex Spyvee, Hampstead, NH (US); Jonah Milton Kallenbach, Somerville, MA (US); Ankit Gupta, Cambridge, MA (US); Elana Simon, Cambridge, MA (US); Gabriel J. Grand, Cambridge, MA (US)

(21) Appl. No.: 17/744,228

(22) Filed: May 13, 2022

Related U.S. Application Data
(63) Continuation of application No. PCT/US2020/061796, filed on Nov. 23, 2020.
(60) Provisional application No. 62/939,283, filed on Nov. 22, 2019.

(51) Int. Cl. C07D 417/12; A61K 31/428; A61K 45/06; C07D 277/84; A61K 31/496; A61K 31/5377; A61K 31/454

(52) U.S. Cl. CPC C07D 417/12; A61K 31/428; A61K 45/06; C07D 277/84; A61K 31/496; A61K 31/5377; A61K 31/454

(57) ABSTRACT

The present disclose includes, among other things, compounds that inhibit CDK9, pharmaceutical compositions and methods of making and using the same.

### RELATED APPLICATIONS

This application is a continuation of International Application No. PCT/US2020/061796, filed Nov. 23, 2020, which claims priority to U.S. Provisional Application No. 62/939,283, filed Nov. 22, 2019, each of which are incorporated herein by reference in their entirety.

### BACKGROUND

Cyclin-dependent protein kinases (CDKs) represent a family of serine/threonine protein kinases that become active upon binding to a cyclin regulatory partner. CDK/cyclin complexes were first identified as regulators of cell cycle progression. More recently however, CDK/cyclin complexes have also been implicated in transcription and mRNA processing. CDK9/PTEFb (positive transcription elongation factor b) phosphorylates the carboxyl-terminal domain (CTD) of the large subunit of RNA polymerase II (RNAP II), predominantly Ser-2, regulating elongation of transcription. Inhibition of CDK9 and transcriptional repression results in the rapid depletion of short lived mRNA transcripts and associated proteins including Mcl-1 and c-myc, leading to induction of apoptosis in tumor cells hyper dependent on these survival proteins. Targeting transcriptional CDKs including CDK9, therefore, represents a therapeutic strategy for treating tumor types hyper dependent on these labile pro-survival proteins including, but not limited to, hematological malignancies such as acute myeloid leukemia, multiple myeloma, chronic lymphocytic leukemia, diffuse large B cell lymphoma. Burkitt's lymphoma, follicular lymphoma and solid tumors such as breast cancer, lung cancer, neuroblastoma and colon cancer. CDK9 inhibitors may also have therapeutic utility in other disease indications including cardiology, virology, inflammation and pain. Therefore there remains a need to develop novel inhibitors of CDKs.

### SUMMARY

The present disclosure provides for compounds of formula (I):

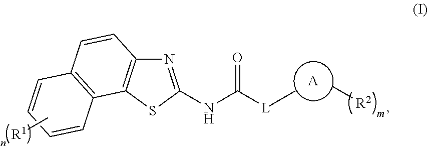

or a pharmaceutically acceptable salt thereof. Additionally, the present disclosure includes, among other things, pharmaceutical compositions, methods of using and methods of making a compound of formula (I).

### DETAILED DESCRIPTION

In some embodiments, the present disclosure includes a compound of Formula (I):

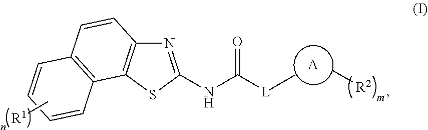

or a pharmaceutically acceptable salt thereof,
wherein
L is selected from a group consisting of a bond, an optionally substituted C₁-C₃ alkylene chain, or —C(H)═C(H)—;
Ring A is selected from the group consisting of optionally substituted C₃-C₆ carbocyclyl, optionally substituted phenyl, optionally substituted naphthyl, optionally substituted 5-10-membered heteroaryl containing 1-3 heteroatoms selected from the group consisting of N, O, and S, and optionally substituted 5-10-membered heterocyclyl containing 1-3 heteroatoms selected from the group consisting of N, O, and S;
each R¹ is independently selected from the group consisting of halogen, —CN, —OR^{a}, —NR^{a}R^{b}, optionally substituted C₁-C₆ alkyl, C₁-C₃ haloalkyl, optionally substituted C₁-C₆ alkoxy, and C₁-C₃haloalkoxy;
each R² is independently selected from the group consisting of halogen, oxo, —CN, —OH, —NR^{a}R^{b}, —NR^{a}C(O)R^{d}, —S(O)₂R^{c}, —NR^{a}S(O)₂R^{c}, —C(O)R^{d}, —C(O)OH, —C(O)OR^{d}, optionally substituted C₁-C₆ alkyl, C₁-C₃ haloalkyl, optionally substituted C₁-C₆ alkoxy, C₁-C₃ haloalkoxy, optionally substituted phenyl, optionally substituted 5-6-membered heteroaryl containing 1-3 heteroatoms selected from the group consisting of N, O, and S, and optionally substituted 5-6-membered heterocyclyl containing 1-3 heteroatoms selected from the group consisting of N, O, and S;
   R^{a} is hydrogen or optionally substituted C₁-C₆ alkyl;
   R^{b} is hydrogen or optionally substituted C₁-C₆ aliphatic;
   R^{c} is optionally substituted C₁-C₆ aliphatic or optionally substituted phenyl;
   R^{d} is optionally substituted C₁-C₆ alkyl, C₁-C₃ haloalkyl, optionally substituted phenyl, optionally substituted benzyl or —O(optionally substituted C₁-C₆ alkyl);
n is 0, 1, 2, or 3; and
m is 0, 1, 2, or 3.

In some embodiments, present disclosure includes a compound of formula (I-a):

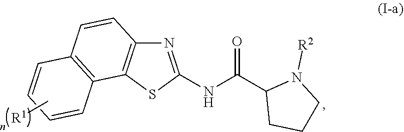

or a pharmaceutically acceptable salt thereof, wherein R¹, R², and n are defined above and described in classes and subclasses herein.

In some embodiments, present disclosure includes a compound of formula (I-b):

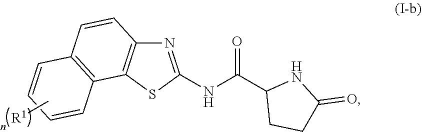

or a pharmaceutically acceptable salt thereof, wherein R¹ and n are defined above and described in classes and subclasses herein.

In some embodiments, Ring A is selected from the group consisting of phenyl, pyrazine, piperidine, morpholine, tetrahydrofuran, C₂-C₆ carbocyclyl, pyrrolidine, pyrrolidone, pyrazole and benzofuran. In some embodiments, Ring A is selected from the group consisting of phenyl, pyrazine, piperidine, morpholine, tetrahydrofuran, C₂-C₆ carbocyclyl, pyrazole and benzofuran. In some embodiments, Ring A is selected from the group consisting of:

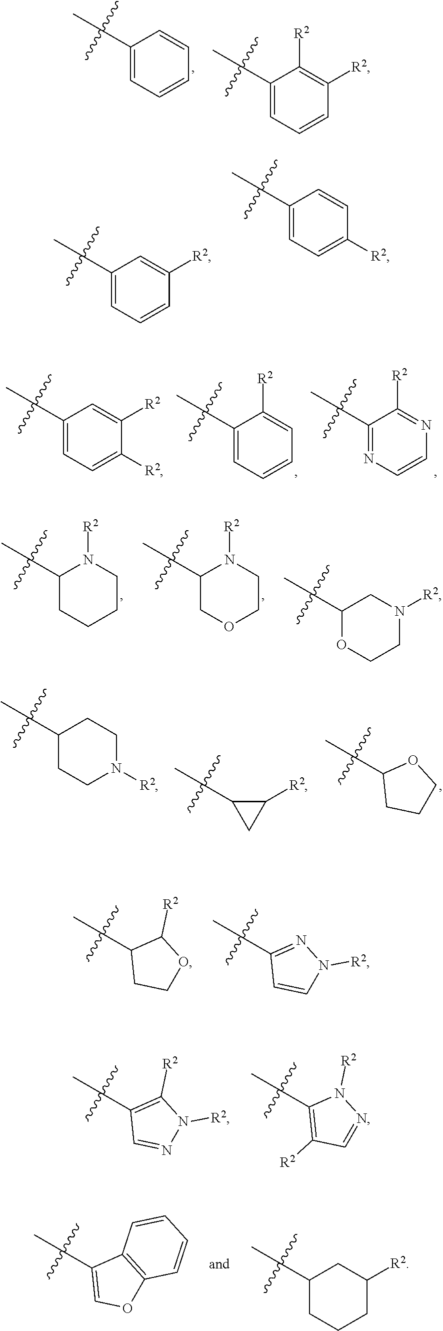

In some embodiments, Ring A is selected from the group consisting of:

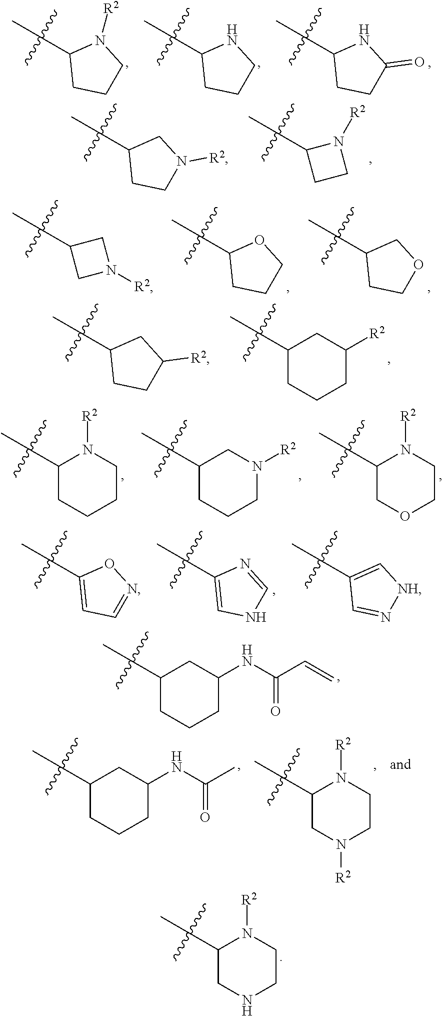

In some embodiments, each R¹ is independently selected from the group consisting of C₁-C₆ aliphatic, halogen, and —CO₂R^{b}. In some embodiments, each R¹ independently is C₁-C₃ aliphatic or halogen. In some embodiments, each R¹ is independently halogen.

In some embodiments, each R² is independently selected from the group consisting of hydrogen, optionally substituted C₁-C₆ aliphatic, optionally substituted C₁-C₃ haloaliphatic, 5-6-membered heterocyclyl containing 1-3 heteroatoms selected from the group consisting of N, O, and S, —C(O)NR^{a}R^{b}, —NR^{a}R^{b}, —S(O)₂R^{c}, and —C(O)R^{d}. In some embodiments, each R² is independently —S(O)₂R^{c}, or —C(O))R^{d}. In some embodiments, R² is hydrogen. In some embodiments, each R² is independently selected from the group consisting of:

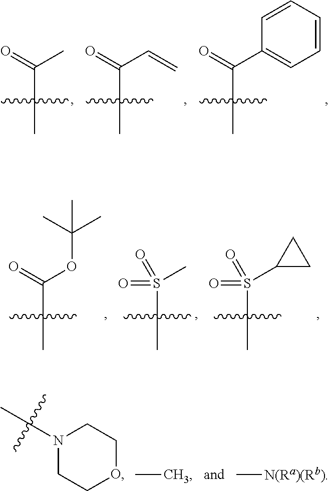

In some embodiments, each Ra is independently hydrogen or optionally substituted C1-C6 aliphatic. In some embodiments, each Ra is independently optionally substituted C1-C6 aliphatic. In some embodiments, each Ra is —CH3. In some embodiments, each Ra is —C(O)CH3. In some embodiments, each Ra is —C(O)CH═CH2. In some embodiments, Ra is hydrogen.

In some embodiments, each Rb is independently hydrogen or optionally substituted C1-C6 aliphatic. In some embodiments, each Rb is independently optionally substituted C1-C6 aliphatic. In some embodiments, each Ra is —CH3. In some embodiments, Rb is hydrogen.

In some embodiments, each Rc is independently optionally substituted C1-C6 aliphatic.

In some embodiments, each Rd is independently optionally substituted C1-C6 aliphatic.

In some embodiments, m is 0. In some embodiments, m is 1. In some embodiments. m is 2. In some embodiments, m is 3.

In some embodiments, n is 0. In some embodiments, n is 1. In some embodiments, n is 2. In some embodiments, n is 3.

In some embodiments, the present disclosure includes, among other things, a compound selected from the group consisting of those described in Table 1:

TABLE 1

| Compound No. | Structure |
|---|---|
| 1 | 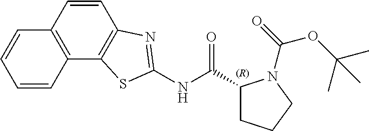 |
| 2 | 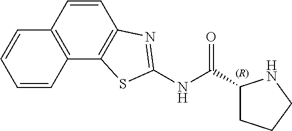 |
| 3 | 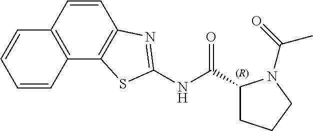 |
| 4 | 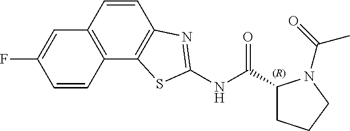 |
| 5 | 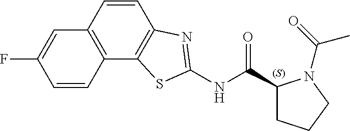 |
| 6 | 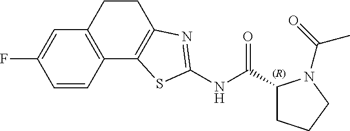 |
| 7 | 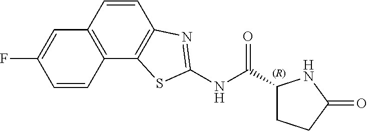 |
| 8 | 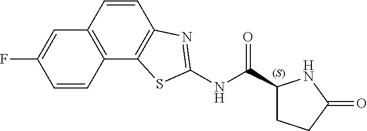 |
| 9 | 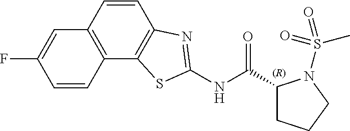 |
| 10 | 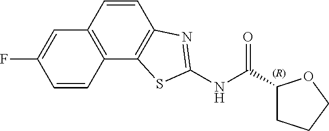 |
| 11 | 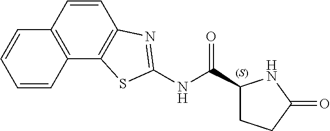 |
| 12 | 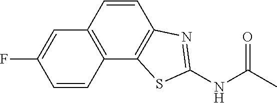 [SMILES: CC(=O)Nc1nc2ccc3cc(F)ccc3c2s1] |
| 13-a | 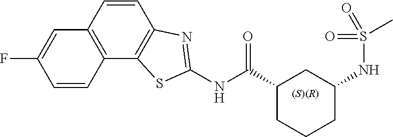 |
| 13-b | 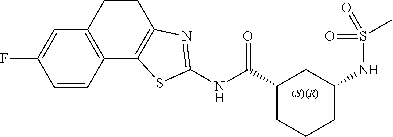 |
| 14 | 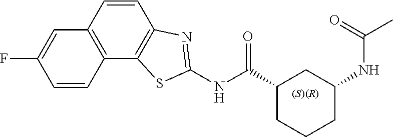 |
| 15 | 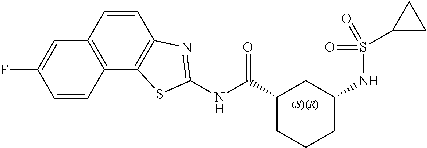 |
| 16 | 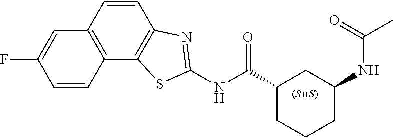 |
| 17 | 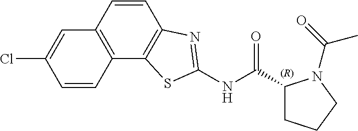 |
| 18-a* | 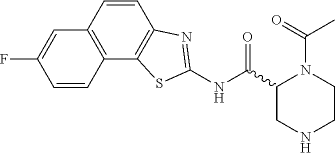 [SMILES: CC(=O)N1CCNCC1C(=O)Nc1nc2ccc3cc(F)ccc3c2s1] |
| 18-b* | 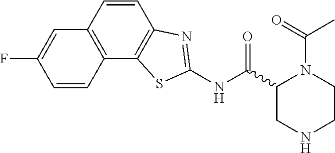 [SMILES: CC(=O)N1CCNCC1C(=O)Nc1nc2ccc3cc(F)ccc3c2s1] |
| 19 | 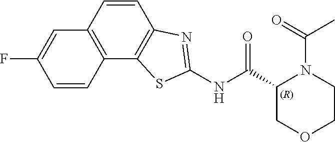 |
| 20 | 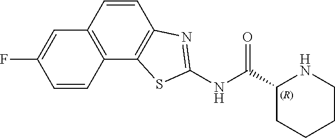 |
| 21 | 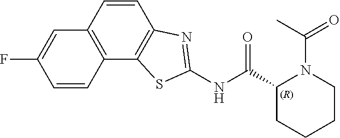 |
| 22 | 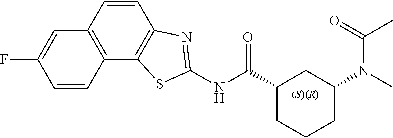 |
| 23 | 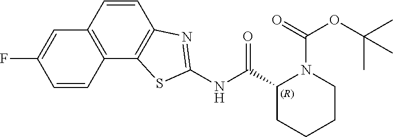 |
| 24 | 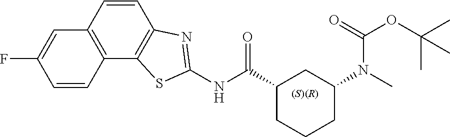 |
| 25 | 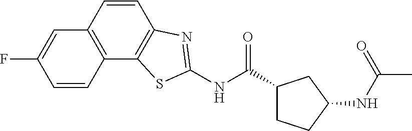 [SMILES: CC(=O)N[C@@H]1CC[C@H](C(=O)Nc2nc3ccc4cc(F)ccc4c3s2)C1] |
| 26 | 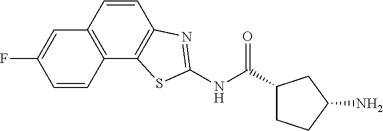 [SMILES: N[C@@H]1CC[C@H](C(=O)Nc2nc3ccc4cc(F)ccc4c3s2)C1] |
| 27 | 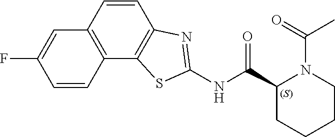 |
| 28 | 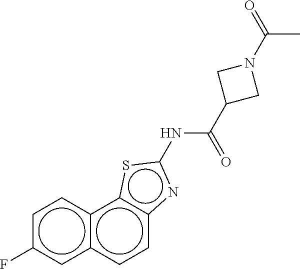 |
| 29 | 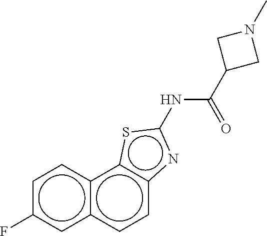 |
| 30 | 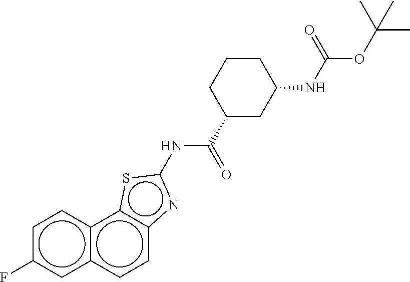 |
| 31 | 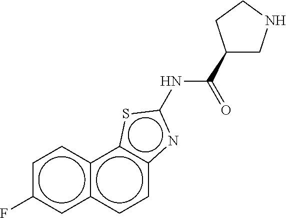 |
| 32 | 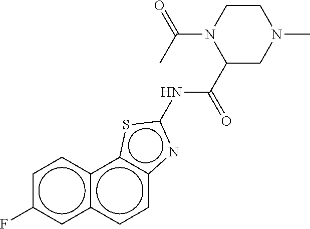 |
| 33 | 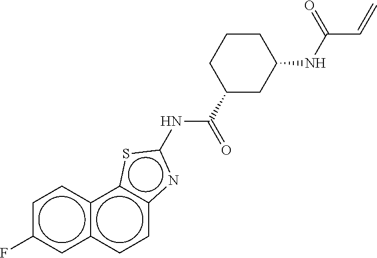 |
| 34 | 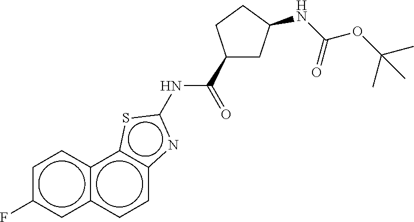 |
| 35 | 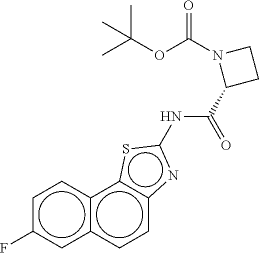 |
| 36 | 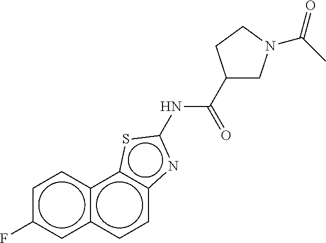 |
| 37 | 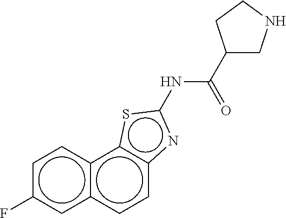 |
| 38 | 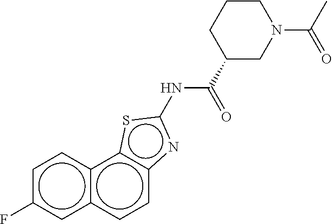 |
| 39 | 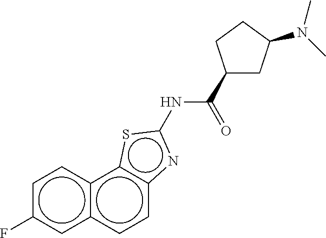 |
| 40 | 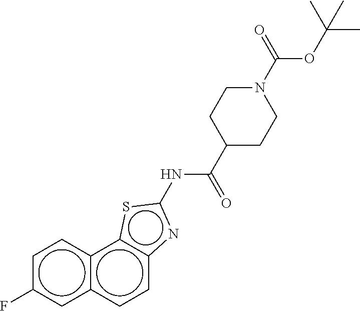 |
| 41 | 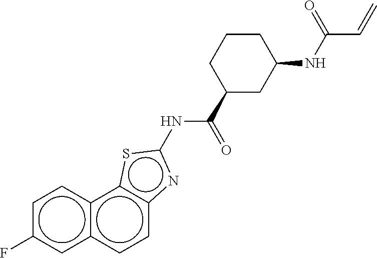 |
| 42 | 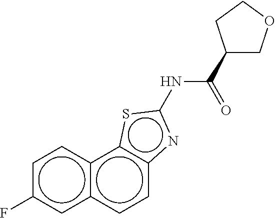 |
| 43 | 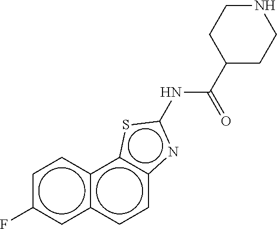 |
| 44 | 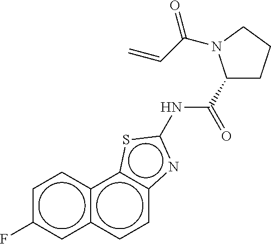 |
| 45 | 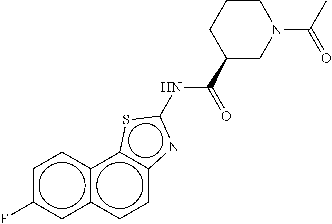 |
| 46 | 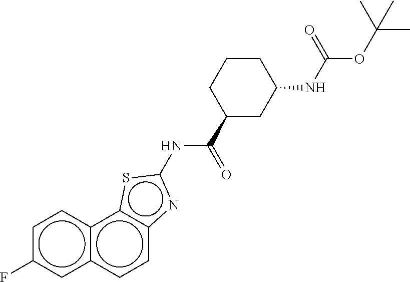 |
| 47 | 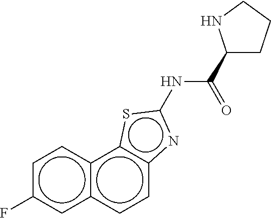 |
| 48 | 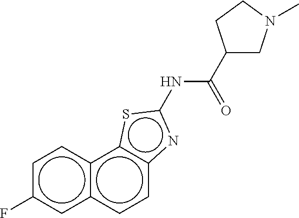 |
| 49 | 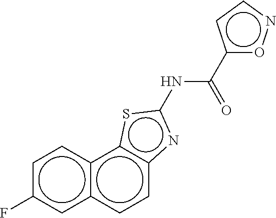 |
| 50 | 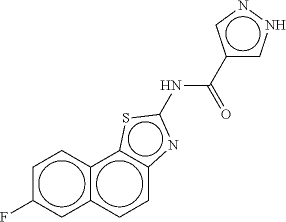 |
| 51 | 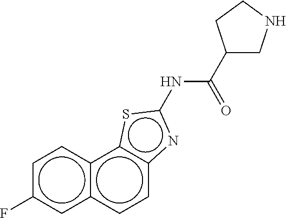 |
| 52 | 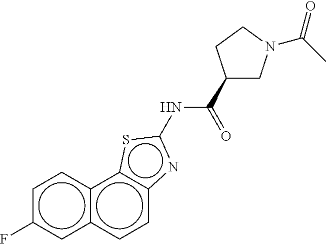 |
| 53 | 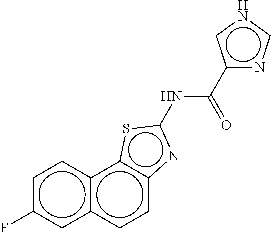 |
| 54 | 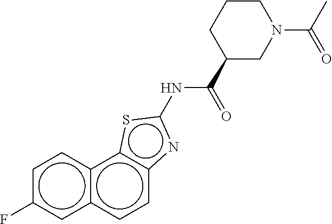 |
| 55 | 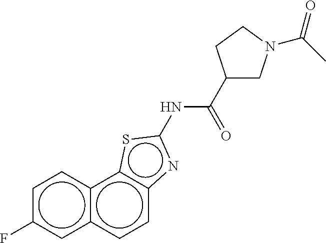 |
| 57 | 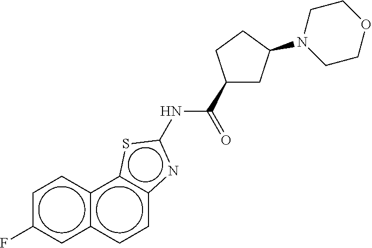 |
| 58 | 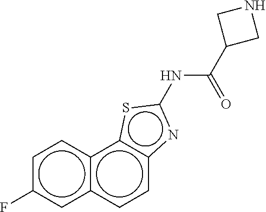 |
| 59 | 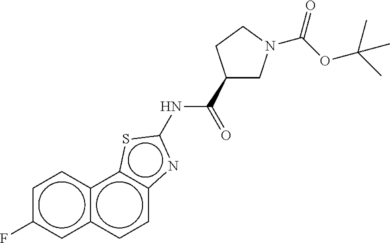 |
| 60 | 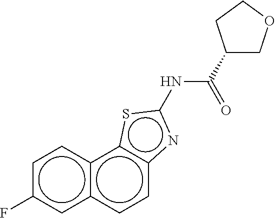 |
| 61 | 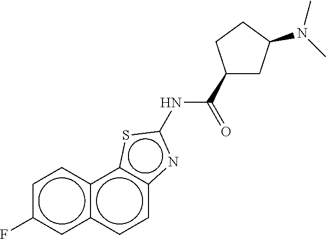 |
| 62 | 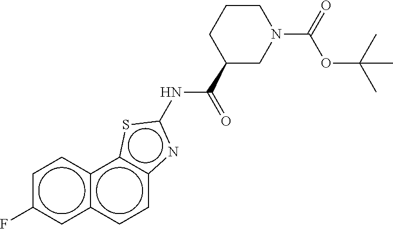 |
| 63 | 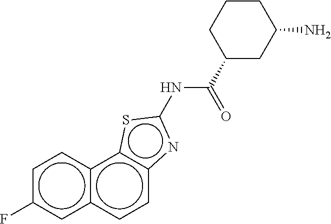 |
| 64 | 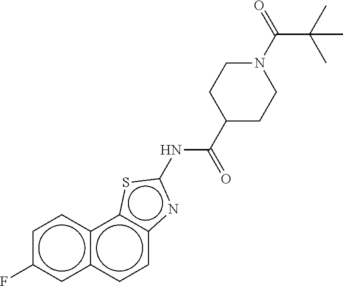 |
| 65 | 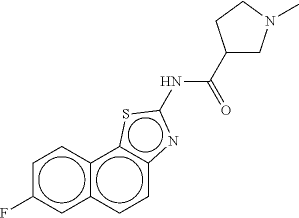 |
| 66 | 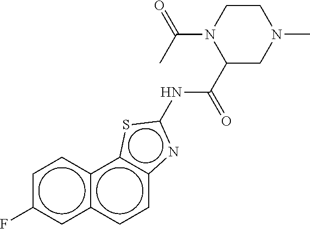 |
| 67 | 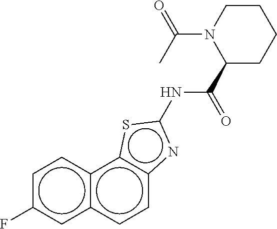 |
| 68 | 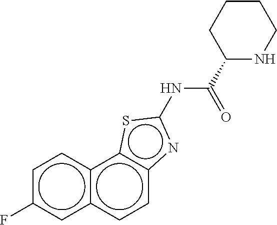 |
| 69 | 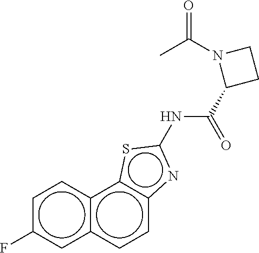 |
| 70 | 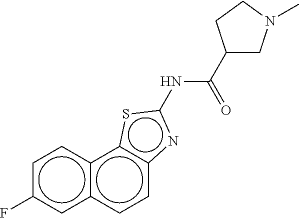 |
| 71 | 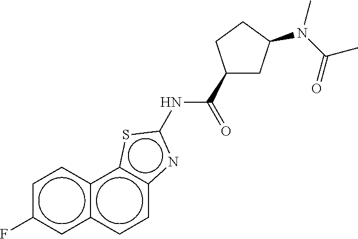 |
| 72 | 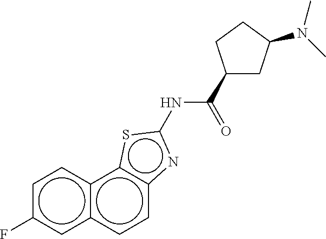 |
| 73 | 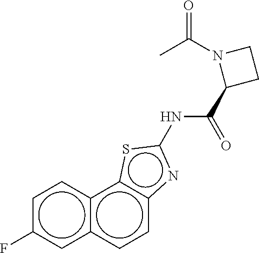 |
| 74 | 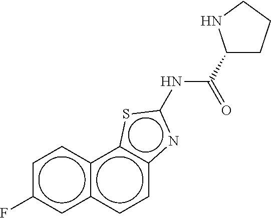 |
| 75 | 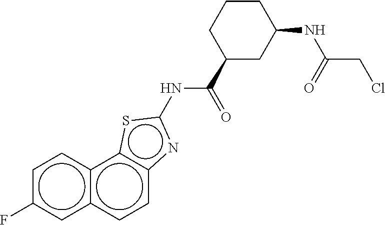 |
| 76 | 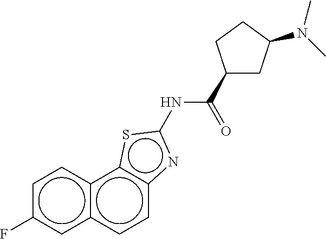 |
| 77 | 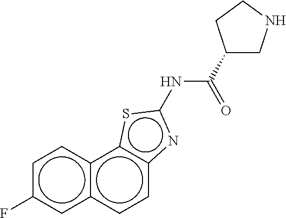 |
| 78 | 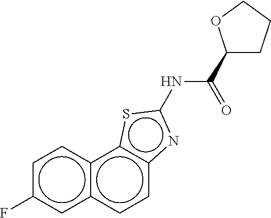 |
| 79 | 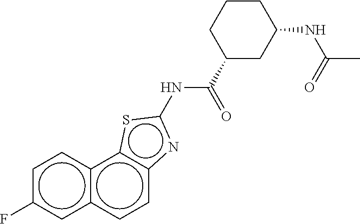 |
| 80 | 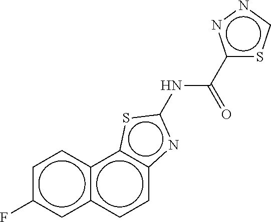 |
| 81 | 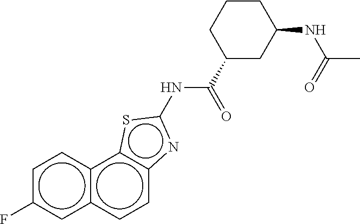 |
| 82 | 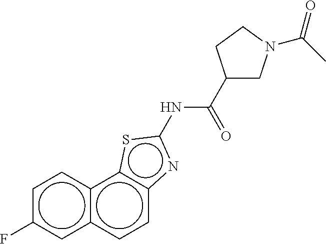 |
| 83 | 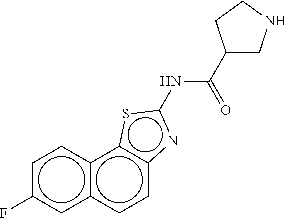 |
| 84 | 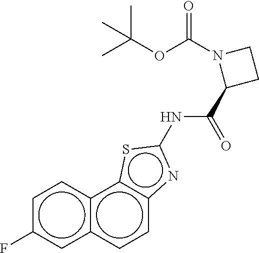 |
| 85 | 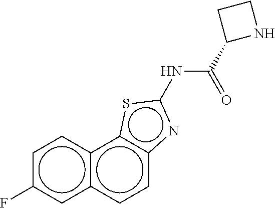 |
| 86 | 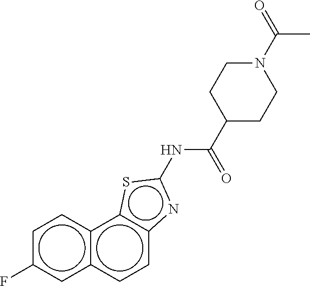 |
| 87 | 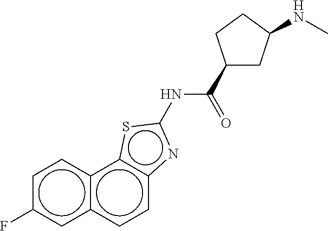 |
| 88 | 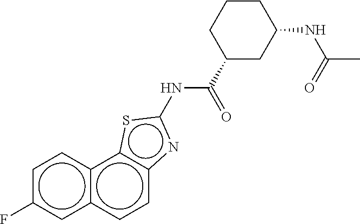 |
| 89 | 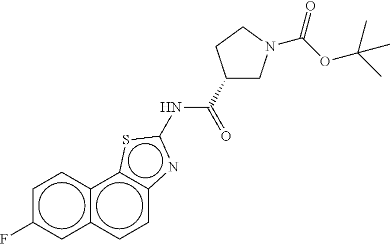 |
| 90 | 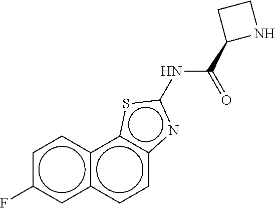 |
| 91 | 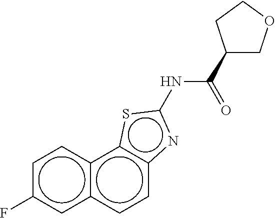 |
| 92 | 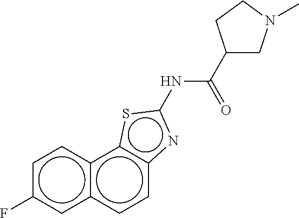 |

*Compounds 18-a and 18-b are enantiomeric pairs of undetermined absolute stereochemistry

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments. “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl. (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of live to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”. “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH2)0-4R∘; —(CH2)0-4OR∘; —O(CH2)0-4R∘, —O—(CH2)0-4C(O)OR∘; —(CH2)0-4CH(OR∘)2; —(CH2)0-4SR∘; —(CH2)0-4Ph, which may be substituted with R∘; —(CH2)0-4O(CH2)0-1Ph which may be substituted with R∘; —CH═CHPh, which may be substituted with R∘; —(CH2)0-4O(CH2)0-1-pyridyl which may be substituted with R∘; —NO2; —CN; —N3; —(CH2)0-4N(R∘)2; —(CH2)0-4N(R∘)C(O)R∘; —N(R∘)C(S)R∘; —(CH2)0-4N(R∘)C(O)NR∘2; —N(R∘)C(S)NR∘2; —(CH2)0-4N(R∘)C(O)OR∘; —N(R∘)N(R∘)C(O)R∘; —N(R∘)N(R∘)C(O)NR∘2; —N(R∘)N(R∘)C(O)OR∘; —(CH2)0-4C(O)R∘; —C(S)R∘; —(CH2)0-4C(O)OR∘; —(CH2)0-4C(O)SR∘; —(CH2)0-4C(O)OSiR∘3; —(CH2)0-4OC(O)R∘; —OC(O)(CH2)0-4SR∘, SC(S)SR∘; —(CH2)0-4SC(O)R∘; —(CH2)0-4C(O)NR∘2; —C(S)NR∘2; —C(S)SR∘; —SC(S)SR∘, —(CH2)0-4OC(O)NR∘2; —C(O)N(OR∘)R∘; —C(O)C(O)R∘; —C(O)CH2C(O)R∘; —C(NOR∘)R∘; —(CH2)0-4SSR∘; —(CH2)0-4S(O)2R∘; —CH2)0-4S(O)2OR∘; —(CH2)0-4OS(O)2R∘; —S(O)2NR∘2; —(CH2)0-4S(O)R∘; —N(R∘)S(O)2NR∘2; —N(R∘)S(O)2R∘; —N(OR∘)R∘; —C(NH)NR∘2; —P(O)2R∘; —P(O)R∘2; —OP(O)R∘2; —OP(O)(OR∘)2; SiR∘3; —(C1-4 straight or branched alkylene)O—N(R∘)2; or —(C1-4 straight or branched alkylene)C(O)O—N(R∘)2, wherein each R∘ may be substituted as defined below and is independently hydrogen, C₁₋₆ aliphatic, —CH2Ph, —O(CH2)0-1Ph, —CH2-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R∘, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R∘ (or the ring formed by taking two independent occurrences of R∘ together with their intervening atoms), are independently halogen, —(CH2)0-2R●, -(haloR●), —(CH2)0-2OH, —(CH2)0-2OR●, —(CH2)0-2CH(OR●)2; —O(haloR●), —CN, —N3, —(CH2)0-2C(O)R●, —(CH2)0-2C(O)OH, —(CH2)0-2C(O)OR●, —(CH2)0-2SR●, —(CH2)0-2SH, —(CH2)0-2NH2, —(CH2)0-2NHR●, —(CH2)0-2NR●2, —NO2, —SiR●3, —OSiR●3, —C(O)SR●, —(C1-4 straight or branched alkylene)C(O)OR●, or —SSR● wherein each Re is unsubstituted or where preceded by “halo” is substituted only with one or more halogens, and is independently selected from C1-4 aliphatic, —CH2Ph, —O(CH2)0-1Ph, or a 5-6-membered saturated, partially unsaturated, or aryl ring having 0-4 heteroatoms independently selected from nitrogen, oxygen, or sulfur. Suitable divalent substituents on a saturated carbon atom of R∘ include ═O and ═S.

Suitable divalent substituents on a saturated carbon atom of an “optionally substituted” group include the following: ═O, ═S, ═NNR*2, ═NNHC(O)R*, ═NNHC(O)OR*, ═NNHS(O)2R*, ═NR*, ═NOR*, —O(C(R*2))2-3O—, or —S(C(R*2))2-3S—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)2-3O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R●, -(haloR●), —OH, —OR●, —O(haloR●), —CN, —C(O)OH, —C(O)OR●, —NH2, —NHR●, —NR●2, or —NO2, wherein each R● is unsubstituted or where preceded by “halo” is substituted only with one or more halogens, and is independently C1-4 aliphatic, —CH2Ph, —O(CH2)0-1Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, —NR†2, —C(O)R†, —C(O)OR†, —C(O)C(O)R†, —C(O)CH2C(O)R†, —S(O)2R†, —S(O)2NR†2, —C(S)NR†2, —C(NH)NR†2, or —N(R†)S(O)2R†; wherein each R† is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R●, -(haloR●), —OH, —OR●, —O(haloR●), —CN, —C(O)OH, —C(O)OR●, —NH2, —NHR●, —NR●2, or —NO2, wherein each R● is unsubstituted or where preceded by “halo” is substituted only with one or more halogens, and is independently C1-4 aliphatic, —CH2Ph, —O(CH2)0-1Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Inhibition of activity of a protein kinase, for example, CDK9 or a mutant thereof, in a biological sample is useful for a variety of purposes that are known to one of skill in the art. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an CDK9-mediated disease or disorder.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably inhibit a protein kinase, particularly at CDK9, or a mutant thereof, in a biological sample or in a patient. In certain embodiments, the amount of compound in compositions of this disclosure is such that is effective to measurably inhibit at CDK9, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a CDK9-mediated disease or condition in a patient comprising the step of administering to said patient a composition according to the present disclosure.

The term “CDK9-mediated disease”, as used herein means any disease or other deleterious condition in which a CDK9 kinase is known to play a role. Accordingly, another embodiment of the present disclosure relates to treating or lessening the severity of one or more diseases in which CDK9 is known to play a role.

In some embodiments, compounds and compositions, according to a method of the present disclosure, may be administered using any amount and any route of administration effective for treating or lessening the severity of cancer, an autoimmune disorder, a neurodegenerative or neurological disorder, schizophrenia, a bone-related disorder, liver disease, or a cardiac disorder. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is selected from the group consisting of non-small cell lung carcinoma, prostate carcinoma, pancreatic ductal adenocarcinoma, cervical carcinoma, melanoma comprising, glioma, acute myeloid leukemia, multiple myeloma, chronic lymphocytic leukemia, diffuse large B cell lymphoma, Burkitt's lymphoma, follicular lymphoma breast cancer, lung cancer, neuroblastoma and colon cancer. In some embodiments, compounds of the present disclosure can be used in a method of treating or lessening the severity of a disease or condition selected from hematological malignancies, such as, acute myeloid leukemia, multiple myeloma, chronic lymphocytic leukemia, diffuse large B cell lymphoma, Burkitt's lymphoma, follicular lymphoma and solid tumors such as breast cancer, lung cancer, neuroblastoma, glioma and colon cancer. In some embodiments cancer is glioma. In some embodiments, a disorder is cancer. In some embodiments, cancer is pancreatic ductal adenocarcinoma.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, the present disclosure relates to a method of inhibiting protein kinase activity in a biological sample comprising the step of contacting said biological sample with a compound of this disclosure, or a composition comprising said compound.

In some embodiments, the present disclosure relates to a method of inhibiting CDK9, or a mutant thereof, activity in a biological sample comprising the step of contacting said biological sample with a compound of this disclosure, or a composition comprising said compound.

Inhibition of CDK9, or a mutant thereof, activity in a biological sample is useful for a variety of purposes that are known to one of skill in the art. Examples of such purposes include, but are not limited to, blood transfusion, organ transplantation, biological specimen storage, and biological assays.

In some embodiments, the present disclosure relates to a method of inhibiting protein kinase activity in a patient comprising the step of administering to said patient a compound of the present disclosure, or a composition comprising said compound.

In some embodiments, the present disclosure relates to a method of inhibiting CDK9, or a mutant thereof, activity in a patient comprising the step of administering to said patient a compound of the present disclosure, or a composition comprising said compound. In some embodiments, the present disclosure provides a method for treating a disorder mediated by CDK9, or a mutant thereof, in a patient in need thereof, comprising the step of administering to said patient a compound according to the present disclosure or pharmaceutically acceptable composition thereof. Such disorders are described in detail herein.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### EXEMPLIFICATION

### Example 1, tert-Butyl (R)-2-(naphtho[2,1-d]thiazol-2-ylcarbamoyl)pyrrolidine-1-carboxylate (1)

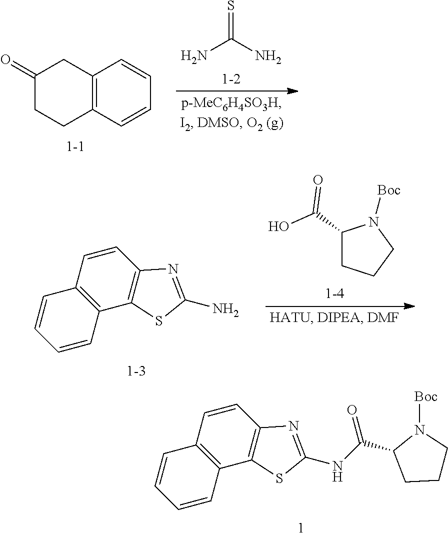

### Naphtho[2,1-d]thiazol-2-amine (1-3)

To a stirred solution of 3,4-dihydronaphthalen-2(1H)-one (1.1, 0.50 g, 3.42 mol) in DMSO (10 mL) were added thiourea (1-2, 0.26 g, 3.42 mmol), PTSA (2.94 g, 17.09 mmol) and iodine (0.26 g, 1.02 mmol) successively. The resulting mixture was stirred 75° C. for 24 h under oxygen atmosphere. The progress of the reaction was monitored by TLC. After completion of the reaction, the reaction mixture was quenched with water (0.30 mL) and extracted with ethyl acetate (3×30 mL). The combined organic layer was washed with brine, dried over anhydrous Na2SO4, filtered and concentrated under reduced pressure resulting in the crude compound. The crude compound was purified over 100-200 mesh silica gel column chromatography (30% ethyl acetate/n-hexane) to afford the title compound 1-3 (0.30 g, 43.85%) as a brown solid. LC-MS: m/z 201.20 [M+H]+.

### tert-Butyl (R)-2-(naphtho[2,1-d]thiazol-2-ylcarbamoyl)pyrrolidine-1-carboxylate (1)

To a stirred solution of (tert-butoxycarbonyl)-D-proline (1-4, 0.32 g, 1.50 mmol) in DMF (3 mL) were added HATU (0.57 g, 1.50 mmol) and DIPEA (0.65 mL, 3.75 mmol) successively at 0° C. The reaction mixture was stirred at 0° C. for 20 min and compound 1-3 (0.30 g, 1.50 mmol) was added. The reaction mixture was then allowed to attain room temperature and stirred for 12 h. The progress of the reaction was monitored by TLC. After completion of the reaction, the reaction mixture was quenched with water (30 mL) and extracted with ethyl acetate (3×20 mL). The combined organic layer was washed with brine, dried over anhydrous Na₂SO₄, filtered and concentrated under reduced pressure resulting in the crude compound. The crude compound was purified over 200-300 mesh silica gel column chromatography (30% ethyl acetate/n-hexane) to afford the title compound 1 (0.25 g, 41.9%) as an off-white solid. ¹H NMR (400 MHz, DMSO-d₆) δ 12.60-12.73 (m, 1H), 8.06 (d, J=8.31 Hz, 2H), 7.93-7.98 (m, 1H), 7.85-7.91 (m, 1H), 7.62-7.66 (m, 1H), 7.52-7.58 (m, 1H), 4.39-4.50 (m, 1H), 3.47 (br s, 1H), 2.26 (d, J=7.83 Hz, 1H), 1.76-2.00 (m, 3H), 1.37-1.44 (m, 3H), 1.30-1.35 (m, 1H), 1.24 (s, 6H); LC-MS: m/z 398.00 [M+H]⁺; HPLC: 96.90%; SOR: 121.86, Solvent: Methanol, Path length: 100 mm, Concentration: 0.25 w/v %.

### Example 2. (R)—N-(naphtho[2,1-d]thiazol-2-yl)pyrrolidine-2-carboxamide (2)

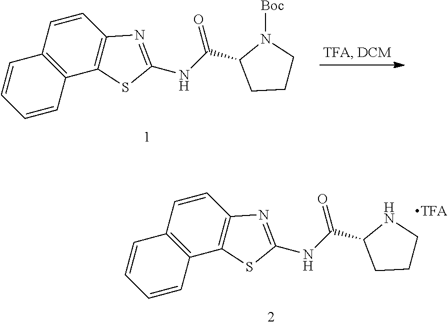 [SMILES: CC(C)(C)OC(=O)N1CCC[C@@H]1C(=O)Nc1nc2ccc3ccccc3c2s1 ; O=C(Nc1nc2ccc3ccccc3c2s1)[C@H]1CCCN1]

### (R)—N-(naphtho[2,1-d]thiazol-2-yl)pyrrolidine-2-carboxamide (2)

To a stirred solution of compound 1 (0.25 g, 0.63 mmol) in DCM (5 mL) was added TFA (1 mL) dropwise at 0° C. The reaction mixture was then allowed to attain room temperature and stirred for 2 h. The progress of the reaction was monitored by TLC. After completion of the reaction, the reaction mixture was concentrated under reduced pressure. The crude compound thus obtained was stirred with diethyl ether and n-hexane successively, filtered and dried under reduced pressure to afford the title compound 2 as TFA salt (0.10 g, 53.4%) as an off white solid. ¹H NMR (400 MHz, DMSO-d₆) δ 12.80-13.46 (m, 1H), 9.43-9.75 (m, 1H), 8.85-9.13 (m, 1H), 8.08 (dd, J=4.16, 7.58 Hz, 2H), 7.96-8.02 (m, 1H), 7.89-7.95 (m, 1H), 7.67 (t, J=7.58 Hz, 1H), 7.54-7.61 (m, 1H), 4.56 (t, J=7.58 Hz, 1H), 3.27-3.40 (m, 2H), 2.39-2.46 (m, 1H), 2.04-2.15 (m, 1H), 1.92-2.03 (m, 2H); LC-MS: m/z 298.35 [M+H]⁺; HPLC: 95.06%; SOR: 58.60, Solvent: DMSO, Path length: 100 mm, Concentration: 0.25 w/v %.

### Example 3. (R)-1-acetyl-N-(naphtho[2,1-d]thiazol-2-yl)pyrrolidine-2-carboxamide (3)

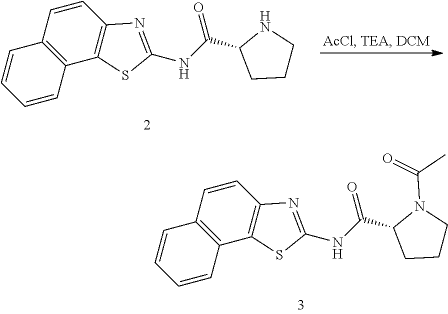 [SMILES: O=C(Nc1nc2ccc3ccccc3c2s1)[C@H]1CCCN1 ; CC(=O)N1CCC[C@@H]1C(=O)Nc1nc2ccc3ccccc3c2s1]

### (R)-1-acetyl-N-(naphtho[2,1-d]thiazol-2-yl)pyrrolidine-2-carboxamide (3)

To a stirred solution of compound 2 (0.10 g, 0.33 mmol) in DCM (2 mL) was added TEA (0.14 mL, 1.01 mmol). The reaction mixture was cooled to 0° C. and AcCl (0.04 g, 0.50 mmol) was added. The reaction mixture was then allowed to attain room temperature and stirred for 2 h. The progress of the reaction was monitored by TLC. After completion of the reaction, the reaction mixture was quenched with ice cooled water and extracted with ethyl acetate (2×25 mL). The combined organic layer was washed with brine, dried over anhydrous Na₂SO₄, filtered and concentrated under reduced pressure resulting in the crude compound. The crude compound was purified by preparative HPLC to afford 3 (0.04 g, 35.0%) as a white solid. ¹H NMR (400 MHz, DMSO-d₆) δ 12.55 (brs, 1H), 8.04 (dd, J=8.07, 13.45 Hz, 2H), 7.92-7.99 (m, 1H), 7.84-7.91 (m, 1H), 7.64 (t, J=7.34 Hz, 1H), 7.50-7.59 (m, 1H), 4.49-4.80 (m, 1H), 3.50-3.61 (m, 2H), 2.27-2.39 (m, 1H), 2.16-2.27 (m, 1H), 2.03 (s, 3H), 1.91-2.00 (m, 2H); LC-MS: m/z 339.85 [M+H]⁺; HPLC: 99.34%; SOR: 56.91, Solvent: DMSO, Path length: 100 mm, Concentration: 0.25 w/v %.

### Example 4. (R)-1-acetyl-N-(7-fluoronaphtho[2,1-d]thiazol-2-yl)pyrrolidine-2-carboxamide (4)

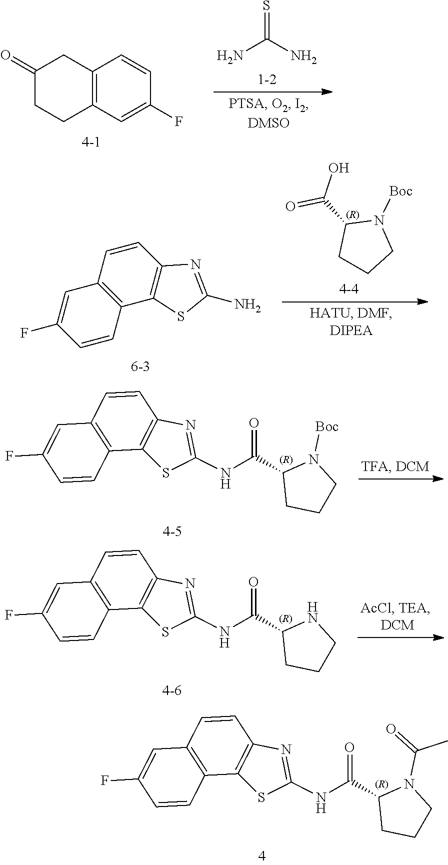

### 7-fluoronaphtho[2,1-d]thiazol-2-amine (4-3)

To a stirred solution of 6-fluoro-3,4-dihydronaphthalen-2(1H)-one (4-1, 2.00 g, 12.18 mmol) and thiourea (1-2, 0.93 g, 12.18 mmol) in DMSO (20 mL), PTSA (10.48 g, 60.90 mmol) and iodine (0.93 g, 3.65 mmol) were added and the reaction mixture was stirred at RT for 24 h in an oxygen atmosphere. The progress of the reaction was monitored by TIC. After completion of the reaction, the reaction mixture was diluted with water. The aqueous layer was extracted with ethyl acetate. The organic layer was dried over anhydrous Na2SO4 and concentrated under reduced pressure. The crude compound was purified by silica gel column chromatography (30% EtOAc/hexane) to afford compound 4-3 (1.0 g, 38.0%) as light brown solid. 1H NMR (400 MHz, DMSO-d6) δ 7.72-7.83 (m, 2H), 7.68 (brs, 1H), 7.62 (d, J=8.31 Hz, 1H), 7.39-7.47 (m, 1H), 2H merged in solvent peak; LC-MS: m/z 219.5 [M+H]+.

### tert-butyl (R)-2-((7-fluoronaphtho[2,1-d]thiazol-2-yl)carbamoyl)pyrrolidine-1-carboxylate (4-5)

To a stirred solution of (tert-butoxycarbonyl)-D-proline (4-4, 0.69 g, 3.21 mmol) in DMF (10 mL), DIPEA (0.89 g, 6.88 mmol) was added followed by HATU (1.46 g, 3.85 mmol) at 0° C. and the reaction mixture was stirred at RT for 30 min. To the resulting reaction mixture, compound 4-3 (0.50 g, 2.23 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was dried over anhydrous Na₂SO₄ and concentrated under reduced pressure. The crude compound was purified by silica gel column chromatography (2% MeOH/DCM) to afford compound 4-5 (0.4 g, 42.0%) as an off white solid. ¹H NMR (400 MHz, DMSO-d₆) δ 12.61-12.75 (m, 1H), 8.09-8.19 (m, 1H), 7.84-7.98 (m, 3H), 7.54 (t, J=8.56 Hz, 1H), 4.37-4.51 (m, 1H), 3.47 (brs, 1H), 3.36 (d, J=3.91 Hz, 1H), 2.18-2.35 (m, 1H), 1.80-1.97 (m, 3H), 1.41 (s, 3H), 1.24 (s, 6H); LC-MS: m/z 416.30 [M+H]⁺.

### (R)—N-(7-fluoronaphtho[2,1-d]thiazol-2-yl)pyrrolidine-2-carboxamide (4-6)

To a stirred solution of compound 4-5 (0.40 g, 0.96 mmol) in DCM (5 mL), TFA (2 mL) was added at 0° C. and the reaction mixture was stirred at RT for 2 h. The progress of the reaction was monitored by TLC. After completion of the reaction, the reaction mixture was concentrated under reduced pressure. The crude compound was triturated with DCM/hexane and dried well to afford compound 4-6 (TFA salt, 0.35 g, crude) as pale yellow solid. LC-MS: m/z 316.08 [M+H]⁺.

### (R)-1-acetyl-N-(7-fluoronaphtho[2,1-d]thiazol-2-yl)pyrrolidine-2-carboxamide (4)

To a stirred solution of compound 4-6 (0.35 g, 0.82 mmol) in DCM (5 ml.) TEA (0.25 g, 2.45 mmol) was added at RT and the reaction mixture was stirred for 15 min. To the resulting reaction mixture, acetyl chloride (0.10 g, 1.22 mmol) was added at 0° C. and the reaction mixture was stirred at RT for 16 h. The progress of the reaction was monitored by TLC. After completion of the reaction, the reaction mixture was quenched with saturated NaHCO₃ solution and the aqueous layer was extracted with DCM. Combined organic layer was dried over anhydrous Na₂SO₄ and concentrated under reduced pressure. The crude compound was purified by prep HPLC to afford 4 (0.05 g, 17.0%) as an off white solid. ¹H NMR (400 MHz, DMSO-d₆) δ 12.56 (brs, 1H), 8.07-8.16 (m, 1H), 7.85-7.98 (m, 3H), 7.54 (t, J=8.80 Hz, 1H), 4.53-4.77 (m, 1H), 3.62-3.71 (m, 1H), 3.51-3.60 (m, 1H), 2.16-2.27 (m, 1H), 2.02 (s, 3H), 1.95 (d, J=6.36 Hz, 2H), 1.85 (s, 1H); LC-MS: m/z 357.95 [M+H]⁺; HPLC: 98.77%; SOR: 67.24, Solvent: DMSO, Path length: 100 mm, Concentration: 0.25 w/v %.

### Example 5. (S)-1-acetyl-N-(7-fluoronaphtho[2,1-d]thiazol-2-yl)pyrrolidine-2-carboxamide (5)

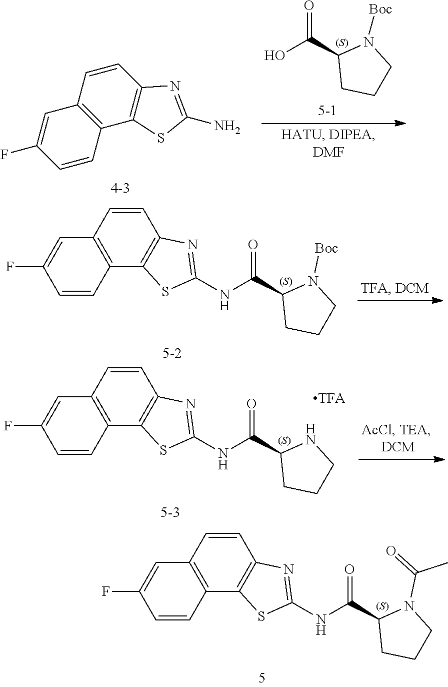

### tert-butyl (S)-2-((7-fluoronaphtho[2,1-d]thiazol-2-yl)carbamoyl)pyrrolidine-1-carboxylate (5-2)

To a stirred solution of compound 4-3 (0.20 g, 0.92 mmol) in DMF (2 mL), DIPEA (0.36 g, 2.75 mmol) and HATU (0.52 g, 1.38 mmol) were added and the reaction mixture was stirred at RT for 10 min. To the resulting reaction mixture, (tert-butoxycarbonyl)-L-proline (5-1, 0.30 g, 1.38 mmol) was added and the reaction mixture was stirred at RT for 16 h. The progress of the reaction was monitored by TLC and LCMS. After completion of the reaction, the reaction mixture was diluted with EtOAc and the aqueous layer was washed with ice cold water. The organic layer was dried over anhydrous Na₂SO₄, filtered and concentrated under reduced pressure. The crude compound was purified by silica gel column chromatography (2% MeOH/DCM) to afford 5-2 (0.15 g, 39.47%) as a white solid. LC-MS: m/z 416.15 [M+H]⁺.

### (S)—N-(7-fluoronaphtho[2,1-d]thiazol-2-yl)pyrrolidine-2-carboxamide (5-3)

To a solution of compound 5-2 (0.15 g, 0.36 mmol) in DCM (2 mL) was added TFA (2 mL) at 0° C. and the reaction mixture was stirred at room temperature for 1 h. The progress of the reaction was monitored by TLC. After completion of the reaction, the reaction mixture was diluted with DCM and washed with NaHCO₃ solution. The organic layer was dried over anhydrous Na₂SO₄, filtered and concentrated under reduced pressure to afford the title compound 5-3 (0.10 g, 88.49%) (TFA salt) as an off-white solid. LC-MS: m/z 316.25[M+H]⁺.

### (S)-1-acetyl-N-(7-fluoronaphtho[2,1-d]thiazol-2-yl)pyrrolidine-2-carboxamide (5)

To a stirred solution of compound 5-3 (0.10 g, 0.32 mmol) in DCM (1 mL) were added Et₃N (0.13 mL, 0.95 mmol) and AcCl (0.04 g, 0.48 mmol) at 0° C. The reaction mixture stirred at same temperature for 3-4 h. The progress of the reaction was monitored by TLC. After completion of the reaction, the reaction mixture was diluted with DCM and water, layers were separated. The organic layer was dried over anhydrous Na₂SO₄, filtered and concentrated under reduced pressure. The crude compound was purified by silica gel column chromatography (5% MeOH/DCM) followed by trituration in hexane to afford the title compound 5 (0.013 g, 11.50%) as a white solid. ¹H NMR (400 MHz, DMSO-d₆) δ 12.55 (brs, 1H), 8.07-8.16 (m, 1H), 7.86-7.98 (m, 3H), 7.50-7.58 (m, 1H), 4.53-4.78 (m, 1H), 3.62-3.71 (m, 1H), 3.51-3.60 (m, 1H), 2.15-2.27 (m, 1H), 2.02 (s, 3H), 1.82-2.00 (m, 3H); LC-MS: m/z 357.95 [M+H]⁺; HPLC: 99.56%; SOR: −55.36; Solvent: DMSO, Path length: 100 mm. Concentration: 0.25 w/v %.

### Example 6. (R)-1-acetyl-N-(7-fluoro-4,5-dihydronaphtho[2,1-d]thiazol-2-yl)pyrrolidine-2-carboxamide (6)

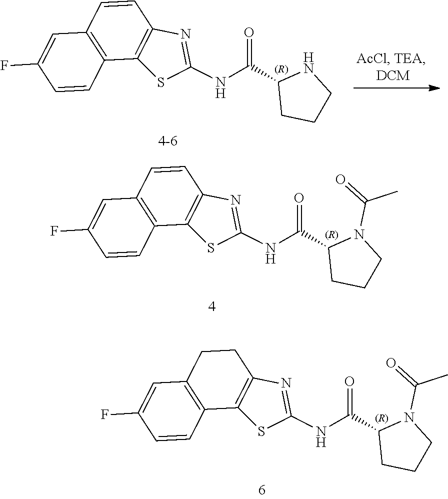

### (R)-1-acetyl-N-(7-fluoro-4,5-dihydronaphtho[2,1-d]thiazol-2-yl)pyrrolidine-2-carboxamide (6)

To a stirred solution of compound 4-6 (TFA salt, 0.19 g, 0.44 mmol) in DCM (2 mL). TEA (0.13 g, 1.33 mmol) was added at RT and the reaction mixture was stirred for 15 min. To the resulting reaction mixture, acetyl chloride (0.0.05 g, 0.66 mmol) was added at 0° C. and the reaction mixture was stirred at RT for 2 h. The progress of the reaction was monitored by TLC. After completion of the reaction, the reaction mixture was quenched with ice cold water and the aqueous layer was extracted with DCM. Combined organic layer was dried over anhydrous Na₂SO₄ and concentrated under reduced pressure. The crude compound was purified by prep HPLC to isolated 4 (0.035 g, 22.0%) along with an isolated impurity 6 (0.01 g, 6.0%) which was a consequence of a corresponding impurity that had been carried forward from the previous synthetic step; both compounds were obtained as off white solids. Analytical details for 6 are as follows: ¹H NMR (400 MHz, DMSO-d₆) δ 12.22 (brs, 1H), 7.21 (dd, J=5.62, 8.56 Hz, 1H), 7.13-7.18 (m, 1H), 7.04 (t, J=8.56 Hz, 1H), 4.46-4.70 (m, 1H), 3.58-3.66 (m, 1H), 3.49-3.57 (m, 1H), 2.96-3.05 (m, 2H), 2.79-2.86 (m, 2H), 2.13-2.23 (m, 1H), 2.00 (s, 3H), 1.80-1.97 (m, 3H); LC-MS: m/z 360.10 [M+H]⁺; HPLC: 99.70.

### Example 7. (R)—N-(7-fluoronaphtho[2,1-d]thiazol-2-yl)-5-oxopyrrolidine-2-carboxamide (7)

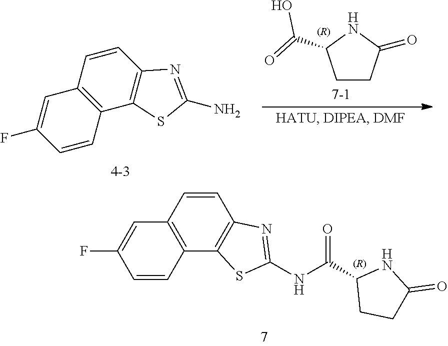

### (R)—N-(7-fluoronaphtho[2,1-d]thiazol-2-yl)-S-oxopyrrolidine-2-carboxamide (7)

To a stirred solution of D-Pyroglutamic acid (7-1, 0.11 g, 0.83 mmol) in DMF (3 mL), DIPEA (0.27 g, 2.06 mmol) was added followed by HATU (0.44 g, 1.15 mmol) at 0° C. and the reaction mixture was stirred at RT for 30 min. To the resulting reaction mixture, compound 4-3 (0.15 g, 0.69 mmol) was added and stirred at 60° C. for 16 h. The progress of the reaction was monitored by TLC. After completion of the reaction, the reaction mixture was diluted with water. The aqueous layer was extracted with ethyl acetate. The organic layer was dried over anhydrous Na₂SO₄ and concentrated under reduced pressure. The crude compound was purified by prep HPLC to afford 7 (0.011 g, 5.0%) as an off white solid. ¹H NMR (400 MHz, DMSO-d₆) δ 12.72 (brs, 1H), 8.16 (dd, J=5.62, 9.05 Hz, 1H), 7.92-7.98 (m, 3H), 7.90 (dd, J=2.45, 10.27 Hz, 1H), 7.51-7.58 (m, 1H), 4.42 (dd, J=4.40, 8.80 Hz, 1H), 2.37-2.46 (m, 1H), 2.13-2.28 (m, 2H), 2.03-2.12 (m, 1H); LC-MS: m/z 329.95 [M+H]⁺; HPLC: 96.27%; SOR: −27.32, Solvent: Methanol, Path length: 100 mm, Concentration: 0.25 w/v %.

### Example 8. (S)—N-(7-fluoronaphtho[2,1-d]thiazol-2-yl)-5-oxopyrrolidine-2-carboxamide (8)

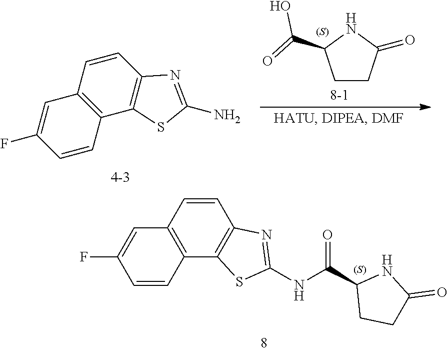

### (S)—N-(7-fluoronaphtho[2,1-d]thiazol-2-yl)-S-oxopyrrolidine-2-carboxamide (8)

To a stirred solution of L-Pyroglutamic acid (8-1, 0.12 g, 0.95 mmol) in DMF (3 mL), DIPEA (0.36 g, 2.75 mmol) was added followed by HATU (0.42 g, 1.10 mmol) at 0° C. and the reaction mixture was stirred at RT for 30 min. To the resulting reaction mixture, compound 4-3 (0.20 g, 0.92 mmol) was added and stirred at 60° C. for 16 h. The progress of the reaction was monitored by TLC. After completion of the reaction, the reaction mixture was diluted with water. The aqueous layer was extracted with ethyl acetate. The organic layer was dried over anhydrous Na₂SO₄ and concentrated under reduced pressure. The crude compound was purified by prep HPLC to afford 8 (0.012 g, 4.0%) as an off white solid. ¹H NMR (400 MHz, DMSO-d₆) δ 12.72 (brs, 1H), 8.16 (dd, J=5.38, 8.80 Hz, 1H), 7.92-7.98 (m, 3H), 7.89 (dd, J=2.45, 10.27 Hz, 1H), 7.50-7.58 (m, 1H), 4.42 (dd, J=3.91, 8.31 Hz, 1H), 2.36-2.45 (m, 1H), 2.14-2.29 (m, 2H), 2.03-2.13 (m, 1H); LC-MS: m/z 329.95 [M+H]⁺; HPLC: 99.86%; SOR: 28.67, Solvent: DMSO, Path length: 100 mm, Concentration: 0.25 w/v %.

### Example 9. (R)—N-(7-fluoronaphtho[2,1-d]thiazol-2-yl)-1-(methylsulfonyl)pyrrolidine-2-carboxamide (9)

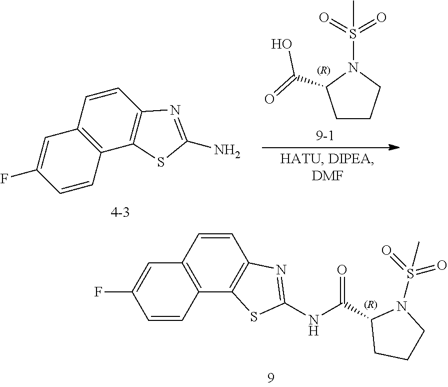

### (R)—N-(7-fluoronaphtho[2,1-d]thiazol-2-yl)-1-(methylsulfonyl)pyrrolidine-2-carboxamide (9)

To a stirred solution of compound 9-1 (0.09 g, 0.45 mmol in DMF (3 mL), DIPEA (0.16 g, 1.24 mmol) was added followed by HATU (0.20 g, 0.54 mmol) at 0° C. and the reaction mixture was stirred at RT for 15 min. To the resulting reaction mixture, compound 4-3 (0.09 g, 0.41 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by prep HPLC to afford 9 (0.013 g, 8.0%) as an off white solid. ¹H NMR (400 MHz, DMSO-d₆) δ 8.00 (dd, J=5.38, 8.80 Hz, 1H), 7.73-7.80 (m, 3H), 7.40-7.49 (m, 1H), 6.07 (brs, 1H), 4.38 (dd, J=3.18, 8.56 Hz, 1H), 3.43-3.52 (m, 2H), 3.04 (s, 3H), 2.16-2.26 (m, 1H), 1.85-2.03 (m, 3H); LC-MS: m/z 393.95 [M+H]⁺; HPLC: 99.40%; SOR: 50.56, Solvent: DMSO, Path length: 100 mm, Concentration: 0.25 w/v %.

### Example 10. (R)—N-(7-fluoronaphtho[2,1-d]thiazol-2-yl)tetrahydrofuran-2-carboxamide (10)

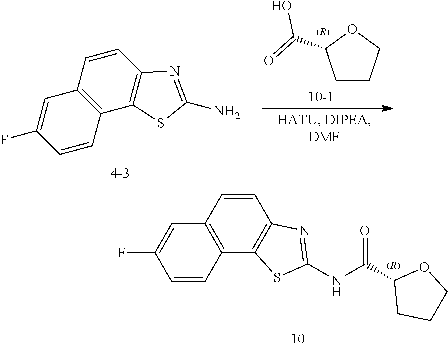

### (R)—N-(7-fluoronaphtho[2,1-d]thiazol-2-yl)tetrahydrofuran-2-carboxamide (10)

To a stirred solution of compound 10-1 (0.05 g, 0.41 mmol) in DMF (2 mL), DIPEA (0.16 g, 1.24 mmol) was added followed by HATU (0.20 g, 0.54 mmol) at 0° C. and the reaction mixture was stirred at RT for 15 min. To the resulting reaction mixture, compound 4-3 (0.09 g, 0.41 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (40% EtOAc/hexane) followed by prep HPLC to afford 10 (0.012 g, 9.0%) as an off white solid. ¹H NMR (400 MHz, DMSO-d₆) δ 8.04-8.15 (m, 1H), 7.79-7.96 (m, 3H), 7.46-7.55 (m, 1H), 6.06 (brs, 1H), 4.55 (dd, J=5.62, 8.07 Hz, 1H), 3.95-4.04 (m, 1H), 3.78-3.86 (m, 1H), 2.15-2.28 (m, 1H), 1.95-2.06 (m, 1H), 1.78-1.94 (m, 2H); LC-MS: m/z 316.95 [M+H]⁺; HPLC: 99.65%; SOR: 27.49, Solvent: Methanol, Path length: 100 mm, Concentration: 0.25 w/v %.

### Example 11. (S)—N-(naphtho[2,1-d]thiazol-2-yl)-5-oxopyrroldine-2-carboxamide (11)

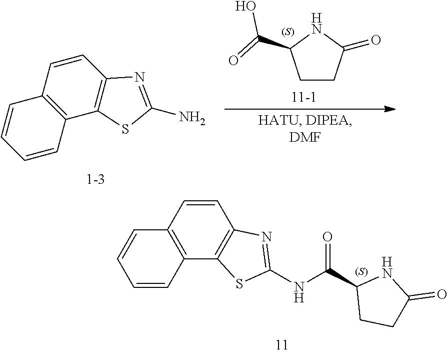

### (S)—N-(naphtho[2,1-d]thiazol-2-yl)-5-oxopyrrolidine-2-carboxamide (11)

To a stirred solution of (S)-5-oxopyrrolidine-2-carboxylic acid (11-1, 0.16 g, 1.25 mmol) in DMF (10 mL), DIPEA (0.48 g, 3.75 mmol) was added followed by HATU (0.57 g, 1.50 mmol) at 0° C. and the reaction mixture was stirred at RT for 30 min. To the resulting reaction mixture, compound 1-3 (0.25 g, 1.25 mmol) was added and the reaction mixture was stirred at RT for 13 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by prep HPLC to afford 11 (0.007 g, 2.0%) as an off white solid. ¹H NMR (400 MHz, DMSO-d₆) δ 8.06 (d, J=8.80 Hz, 2H), 7.91-7.98 (m, 2H), 7.86-7.90 (m, 1H), 7.64 (t, J=7.83 Hz, 1H), 7.52-7.58 (m, 1H), 4.41 (d, J=3.91 Hz, 1H), 2.76 (s, 1H), 2.15-2.26 (m, 2H), 2.05-2.13 (m, 1H), 1H merged in solvent peak; LC-MS: m/z 312.05 [M+H]⁺; HPLC: 93.63%.

### Example 12. N-(7-fluoronaphtho[2,1-d]thiazol-2-yl)acetamide (12)

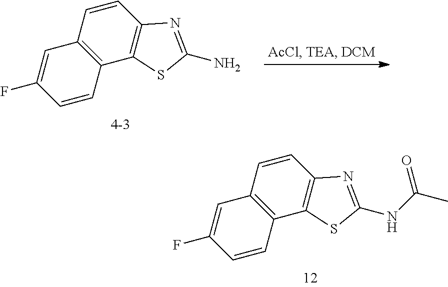 [SMILES: Nc1nc2ccc3cc(F)ccc3c2s1 ; CC(=O)Nc1nc2ccc3cc(F)ccc3c2s1]

### N-(7-fluoronaphtho[2,1-d]thiazol-2-yl)acetamide (12)

To a stirred solution of compound 4-3 (0.15 g, 0.69 mmol) in DCM (3 mL). TEA (0.21 g, 2.06 mmol) was added at RT and the reaction mixture was stirred for 10 min. To the resulting reaction mixture, acetyl chloride (0.08 g, 1.03 mmol) was added at 0° C. and the reaction mixture was stirred at RT for 16 h. The progress of the reaction was monitored by TLC. After completion of the reaction, the reaction mixture was diluted with ice cold water and extracted with DCM. Combined organic layer was dried over anhydrous Na₂SO₄ and concentrated under reduced pressure. The crude compound was purified by prep HPLC to afford 12 (0.025 g, 14.0%) as an off white solid. ¹H NMR (400 MHz, DMSO-d₆) δ 12.50 (brs, 1H), 8.15 (dd, J=5.38, 9.29 Hz, 1H), 7.85-7.96 (m, 3H), 7.49-7.56 (m, 1H), 2.24 (s, 3H); LC-MS: m/z 261.00 [M+H]⁺; HPLC: 97.13%.

### Example 13. (1S,3R)—N-(7-fluoronaphtho[2,1-d]thiazol-2-yl)-3-(methylsulfonamido)cyclohexane-1-carboxamide (13-a) and (1S,3R)—N-(7-fluoro-4,5-dihydronaphtho[2,1-d]thiazol-2-yl)-3-(methylsulfonamido)cyclohexane-1-carboxamide (13-b)

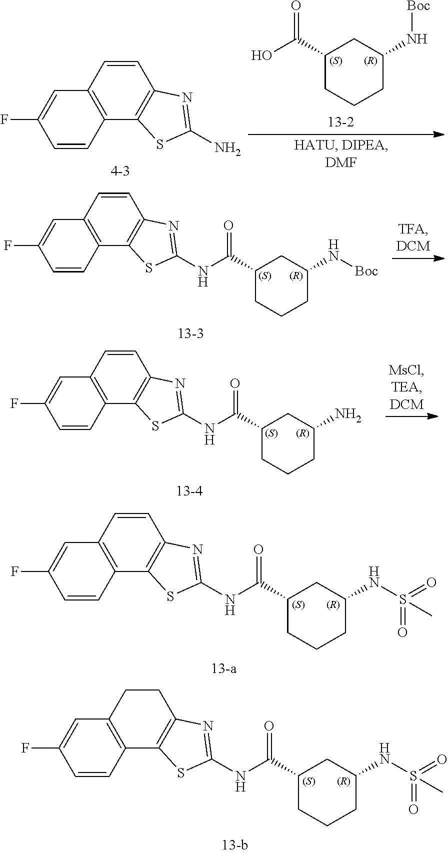

### Tert-butyl ((1R,3S)-3-((7-fluoronaphtho[2,1-d]thiazol-2-yl)carbamoyl)cyclohexyl)carbamate (13-3)

To a stirred solution of compound 4-3 (0.35 g, 1.60 mmol) and (1S,3R)-3-((tert-butoxycarbonyl)amino)cyclohexane-1-carboxylic acid (13-2, 0.58 g, 2.40 mmol) in DMF (5 mL) at 0° C. was added DIPEA (1.5 mL, 8.00 mmol). The reaction mixture was stirred at RT for 12 h. To the resulting reaction mixture, HATU (0.91 g, 2.40 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (15% EtOAc/hexane) to afford compound 13-3 (0.40 g, 89.0%) as an off white solid. LC-MS: m/z 443.8 [M+H]⁺.

### (1S,3R)-3-amino-N-(7-fluoronaphtho[2,1-d]thiazol-2-yl)cyclohexane-1-carboxamide (13-4)

A mixture of compound 13-3 (0.40 g, 0.90 mmol) and 30% TFA/DCM (20 mL) was stirred at 0° C. for 10 min followed by at RT for 12 h. The progress of the reaction was monitored by TLC. After completion of the reaction, the reaction mixture was concentrated under reduced pressure. The crude compound was triturated with diethyl ether and pentane and dried well to afford compound 13-4 (TFA salt, 0.30 g, crude) as an off-white solid which was used as such for the next reaction. LC-MS: m/z 344.27 [M+H]⁺.

### (1S,3R)—N-(7-fluoronaphtho[2,1-d]thiazol-2-yl)-3-(methylsulfonamido)cyclohexane-1-carboxamide (13-a) and (1S,3R)—N-(7-fluoro-4,5-dihydronaphtho[2,1-d]thiazol-2-yl)-3-(methylsulfonamido)cyclohexane-1-carboxamide (13-b)

To a stirred solution of compound 13-4 (0.15 g, 0.43 mmol) in DCM (5 mL), TEA (0.13 g, 1.30 mmol) was added at RT and the reaction mixture was stirred for 10 min. To the resulting reaction mixture, methanesulfonyl chloride (0.07 g, 0.65 mmol was added at 0° C. and the reaction mixture was stirred at RT for 2 h. The progress of the reaction was monitored by TLC. After completion of the reaction, the reaction mixture was diluted with ice cold water and extracted with 5% MeOH/DCM. Combined organic layer was dried over anhydrous Na₂SO₄ and concentrated under reduced pressure. The crude compound was purified by prep HPLC to isolate 13-a (0.04 g, 22.0%) and isolate 13-b (0.006 g, 3.0%) both as an off white solid. 13-b was a consequence of sulfonylation of the corresponding impurity within the starting material.

13-a: ¹H NMR (400 MHz, DMSO-d₆) δ 12.49 (brs, 1H), 8.13 (dd, J=5.62, 9.05 Hz, 1H), 7.85-7.97 (m, 3H), 7.50-7.57 (m, 1H), 7.15 (d, J=7.83 Hz, 1H), 3.14-3.24 (m, 1H), 2.94 (s, 3H), 2.63-2.72 (m, 1H), 2.14 (d, J=12.72 Hz, 1H), 1.78-1.95 (m, 3H), 1.15-1.48 (m, 4H); LC-MS: m/z 422.10 [M+H]⁺; HPLC: 96.19%; SOR: 52.26, Solvent: DMSO, Path length: 100 mm, Concentration: 0.255 w/v %.

13-b: ¹H NMR (400 MHz, DMSO-d₆) δ 12.17 (brs, 1H), 7.22 (dd, J=5.87, 8.31 Hz, 1H), 7.11-7.17 (m, 2H), 7.00-7.06 (m, 1H), 3.10-3.21 (m, 2H), 2.97-3.03 (m, 2H), 2.93 (s, 3H), 2.78-2.85 (m, 2H), 2.07 (d, J=12.23 Hz, 1H), 1.89 (d, J=11.25 Hz, 1H), 1.79 (d, J=10.76 Hz, 2H), 1.14-1.43 (m, 4H); LC-MS: m/z 424.05 [M+H]⁺; HPLC: 97.62%; SOR: 42.76, Solvent: DMSO, Path length: 100 mm, Concentration: 0.255 w/v %.

### Example 14. (1S,3R)-3-acetamido-N-(7-fluoronaphtho[2,1-d]thiazol-2-yl)cyclohexane-1-carboxamide (14)

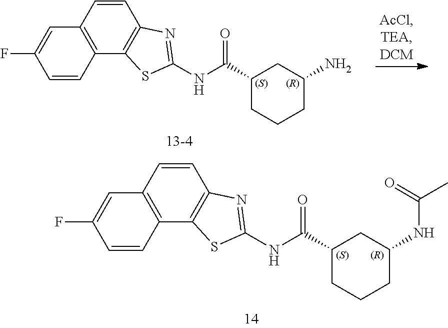

### (1S,3R)-3-acetamido-N-(7-fluoronaphtho[2,1-d]thiazol-2-yl)cyclohexane-1-carboxamide (14)

To a stirred solution of compound 13-4 (0.09 g, 0.26 mmol) in DCM (2 mL). TEA (0.10 mL, 0.78 mmol) was added at RT and the reaction mixture was stirred for 10 min. To the resulting reaction mixture, acetyl chloride (0.02 g, 0.31 mmol) was added at 0° C. and the reaction mixture was stirred at RT for 16 h. The progress of the reaction was monitored by TLC. After completion of the reaction, the reaction mixture was diluted with ice cold water and extracted with DCM. Combined organic layer was dried over anhydrous Na₂SO₄ and concentrated under reduced pressure. The crude compound was purified by silica gel column chromatography (0.30% EtOAc/hexane) followed by prep H-PLC to afford 14 (0.011 g, 11.0%) as an off white solid. ¹H NMR (400 MHz, DMSO-d₆) δ 12.48 (brs, 1H), 8.03-8.19 (m, 1H), 7.67-8.00 (m, 4H), 7.46-7.61 (m, 1H), 3.60 (brs, 1H), 2.68 (brs, 1H), 1.98 (d, J=11.25 Hz, 1H), 1.64-1.92 (m, 6H), 1.19-1.44 (m, 3H), 0.95-1.17 (m, 1H); LC-MS: m/z 385.7 [M+H]⁺; HPLC: 95.35%; SOR: 38.92, Solvent: DMSO, Path length: 100 mm, Concentration: 0.4 w/v %.

### Example 15. (1S,3R)-3-(cyclopropanesulfonamido)-N-(7-fluoronaphtho[2,1-d]thiazol-2-yl)cyclohexane-1-carboxamide (15-a) and (1S,3R)-3-(cyclopropanesulfonamido)-N-(7-fluoro-4,5-dihydronaphtho[2,1-d]thiazol-2-yl)cyclohexane-1-carboxamide (15-b)

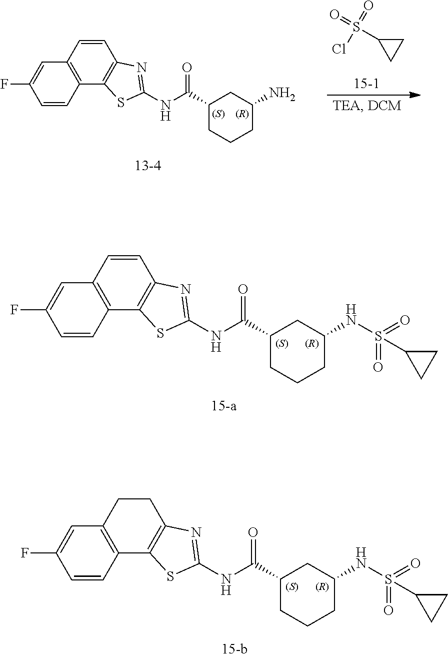

### (1S,3R)-3-(cyclopropanesulfonamido)-N-(7-fluoronaphtho[2,1-d]thiazol-2-yl)cyclohexane-1-carboxamide (15-a) and (1S,3R)-3-(cyclopropanesulfonamido)-N-(7-fluoro-4,5-dihydronaphtho[2,1-d]thiazol-2-yl)cyclohexane-1-carboxamide (15-b)

To a stirred solution of compound 13-4 (0.15 g, 0.43 mmol) in DCM (5 mL), TEA (0.13 g, 1.30 mmol) was added at RT and the reaction mixture was stirred for 10 min. To the resulting reaction mixture, cyclopropanesulfonyl chloride (1, 0.09 g, 0.65 mmol) was added at 0° C. and the reaction mixture was stirred at RT for 4 h. The progress of the reaction was monitored by TLC. After completion of the reaction, the reaction mixture was diluted with ice cold water and extracted with 5% MeOH/DCM. Combined organic layer was dried over anhydrous Na₂SO₄ and concentrated under reduced pressure. The crude compound was purified by prep HPLC to isolate 15-a (0.04 g, 21.0%) and isolate 15-b (0.006 g, 3.0%) both as an off white solid. 15-b formation was a consequence of sulfonylation of the corresponding impurity comprised within the starting material.

15-a: ¹H NMR (400 MHz, DMSO-d₆) δ 12.50 (brs, 1H), 8.10-8.17 (m, 1H), 7.85-7.96 (m, 3H), 7.50-7.57 (m, 1H), 7.18 (d, J=7.83 Hz, 1H), 3.21 (dd, J=3.91, 7.83 Hz, 1H), 2.63-2.72 (m, 1H), 2.54-2.62 (m, 1H), 2.15 (d, J=12.72 Hz, 1H), 1.78-1.98 (m, 3H), 1.19-1.51 (m, 4H), 0.88-0.97 (m, 4H); LC-MS: m/z 448.15 [M+H]⁺; HPLC: 99.84%; SOR: 43.82, Solvent: DMSO, Path length: 100 mm, Concentration: 0.255 w/v %.

15-b: ¹H NMR (400 MHz, DMSO-d₆) δ 12.18 (brs, 1H), 7.19-7.25 (m, 1H), 7.11-7.19 (m, 2H), 6.99-7.07 (m, 1H), 3.13-3.23 (m, 1H), 2.96-3.03 (m, 2H), 2.79-2.85 (m, 2H), 2.55-2.63 (m, 2H), 2.05-2.13 (m, 1H), 1.88-1.97 (m, 1H), 1.74-1.84 (m, 2H), 1.16-1.47 (m, 4H), 0.87-0.98 (m, 4H); LC-MS: m/z 449.95 [M+H]⁺; HPLC: 99.66%; SOR: 47.30, Solvent: DMSO, Path length: 100 mm, Concentration: 0.255 w/v %.

### Example 16. (1S,3S)-3-acetamido-N-(7-fluoronaphtho[2,1-d]thiazol-2-yl)cyclohexane-1-carboxamide (16)

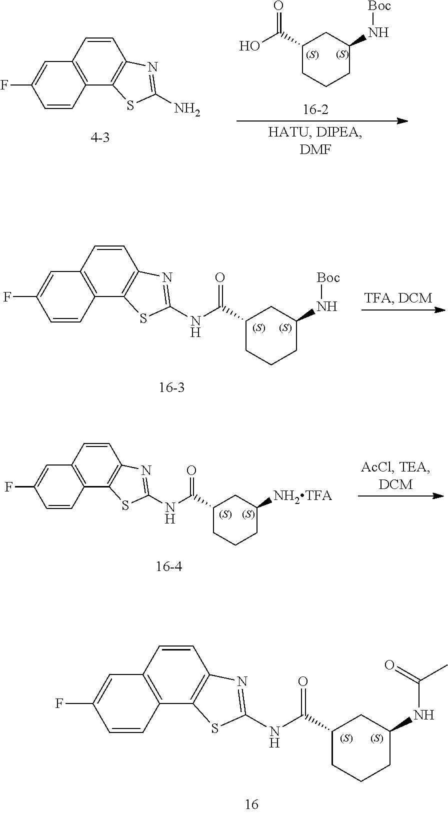

### tert-butyl ((1S,3S)-3-((7-fluoronaphtho[2,1-d]thiazol-2-yl)carbamoyl)cyclohexyl)carbamate (16-3)

To a stirred solution of (1S,3S)-3-((tert-butoxycarbonyl)amino)cyclohexane-1-carboxylic acid (16-2, 0.27 g, 1.10 mmol) in DMF (2 ml), was added DIPEA (0.5 mL, 2.75 mmol), followed by HATU (0.52 g, 1.37 mmol) at 0° C. and the reaction mixture was stirred at RT for 10 min. To the resulting reaction mixture, compound 4-3 (0.20 g, 0.92 mmol) was added and the reaction mixture was stirred at RT for 12 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (25% EtOAc/hexane) to afford compound 16-3 (0.25 g, 61.5%) as an off-white solid. ¹H NMR (400 MHz, DMSO-d₆) δ 12.34 (brs, 1H), 8.13 (dd, J=5.62, 9.05 Hz, 1H), 7.85-7.96 (m, 3H), 7.51-7.58 (m, 1H), 6.65 (d, J=4.40 Hz, 1H), 3.72 (brs, 1H), 2.91 (brs, 1H), 1.49-1.87 (m, 8H), 1.39 (s, 9H); LC-MS: m/z 444.15 [M+H]⁺.

### (1S,3S)-3-amino-N-(7-fluoronaphtho[2,1-d]thiazol-2-yl)cyclohexane-1-carboxamide. TFA salt (16-4)

To a stirred solution of compound 16-3 (0.24 g, 0.54 mmol) in DCM (5 mL), TFA (2 mL) was added drop wise at 0° C. and the reaction mixture was stirred at RT for 2 h. The progress of the reaction was monitored by TLC. After completion of the reaction, the reaction mixture was concentrated under reduced pressure. The crude compound was triturated with diethyl ether followed by hexane and dried well to afford compound 16-4 (TFA salt, 0.20 g, crude) as an off white solid. LC-MS: m/z 345.40 [M+2H]⁺.

### (1S,3S)-3-acetamido-N-(7-fluoronaphtho[2,1-d]thiazol-2-yl)cyclohexane-1-carboxamide (16)

To a stirred solution of compound 16-4 (0.15 g, 0.44 mmol in DCM (2 mL), TEA (0.2 mL, 1.31 mmol) was added at RT and the reaction mixture was stirred for 15 min. To the resulting reaction mixture, acetyl chloride (0.04 g, 0.52 mmol) was added at 0° C. and the reaction mixture was stirred at RT for 2 h. The progress of the reaction was monitored by TLC. After completion of the reaction, the reaction mixture was diluted with ice cold water, aqueous layer was extracted with DCM. Combined organic layer was dried over anhydrous Na₂SO₄ and concentrated under reduced pressure. The crude compound was purified by silica gel column chromatography (3% MeOH/DCM) to afford 16 (0.016 g, 9.5%) as an off white solid. ¹H NMR (400 MHz, DMSO-d₆) δ 12.42 (brs, 1H), 8.13 (dd, J=5.38, 8.80 Hz, 1H), 7.85-7.97 (m, 3H), 7.69 (d, J=6.85 Hz, 1H), 7.50-7.58 (m, 1H), 3.99 (brs, 1H), 2.88 (brs, 1H), 1.85 (s, 3H), 1.72-1.81 (m, 3H), 1.52-1.70 (m, 4H), 1.42-1.51 (m, 1H); LC-MS: m/z 386.10 [M+H]⁺; HPLC: 96.63%; SOR: 24.09, Solvent: Methanol, Path length: 100 mm, Concentration: 0.25 w/v %.

### Example 17. (R)-1-acetyl-N-(7-chloronaphtho[2,1-d]thiazol-2-yl)pyrrolidine-2-carboxamide (17)

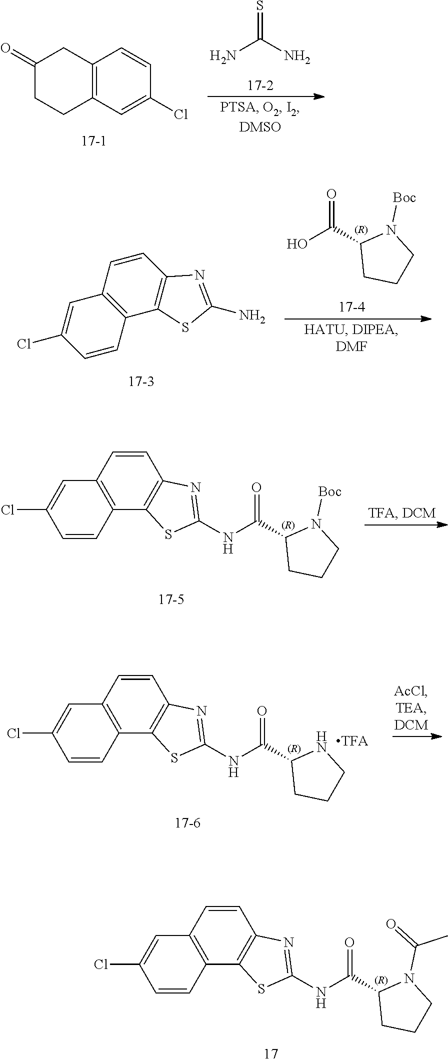

### 7-chloronaphtho[2,1-d]thiazol-2-amine (17-3)

To a stirred solution of 6-chloro-3,4-dihydronaphthalen-2(1H)-one (0.17-1.0.50 g, 2.77 mmol) in DMSO (5 mL), thiourea (17-2, 0.23 g, 2.77 mmol) was added followed by PTSA (2.38 g, 13.88 mmol) and iodine (0.21 g, 0.83 mmol). Oxygen was purged through the reaction mixture for 30 min at RT. The reaction mixture was stirred at 75° C. for 48 h under O₂ atmosphere. The progress of the reaction was monitored by TLC. After completion of the reaction, the reaction mixture was diluted with water. The aqueous layer was extracted with ethyl acetate. Combined organic layer was dried over anhydrous Na₂SO₄ and concentrated under reduced pressure. The crude compound was purified by silica gel column chromatography (20% EtOAc/hexane) to afford compound 17-3 (0.30 g, 46.0%) as brown solid. LC-MS: m/z 235.18 [M+H]⁺.

### tert-butyl (R)-2-((7-chloronaphtho[2,1-d]thiazol-2-yl)carbamoyl)pyrrolidine-1-carboxylate (17-5)

To a stirred solution of (tert-butoxycarbonyl)-D-proline (17-4, 0.12 g, 0.54 mmol) in DMF (5 mL), DIPEA (0.20 mL, 1.08 mmol) was added followed by HATU (0.21 g, 0.54 mmol) at 0° C. and the reaction mixture was stirred at RT for 15 min. To the resulting reaction mixture, compound 17-3 (0.09 g, 0.36 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (30% EtOAc/hexane) to afford compound 17-5 (0.07 g, 45.0%) as an off white solid. LC-MS: m/z 432.30 [M+H]⁺.

### (R)—N-(7-chloronaphtho[2,1-d]thiazol-2-yl)pyrrolidine-2-carboxamide. TFA salt (17-6)

To a stirred solution of compound 17-5 (0.07 g, 0.16 mmol) in DCM (2 mL), TFA (2 mL) was added drop wise at 0° C. and the reaction mixture was stirred at RT for 2 h. The progress of the reaction was monitored by TLC. After completion of the reaction, the reaction mixture was concentrated under reduced pressure. The crude compound was triturated with diethyl ether followed by hexane and dried well to afford compound 17-6 (TFA salt, 0.07 g, crude) as brown solid. LC-MS: m/z 332.02 [M+H]⁺.

### (R)-1-acetyl-N-(7-chloronaphtho[2,1-d]thiazol-2-yl)pyrrolidine-2-carboxamide (17)

To a stirred solution of compound 17-6 (0.07 g, 0.15 mmol) in DCM (2 mL), TEA (0.03 g, 0.31 mmol) was added at RT and the reaction mixture was stirred for 15 min. To the resulting reaction mixture, acetyl chloride (0.02 g, 0.23 mmol) was added at 0° C. and the reaction mixture was stirred at RT for 2 h. The progress of the reaction was monitored by TLC. After completion of the reaction, the reaction mixture was diluted with ice cold water, aqueous layer was extracted with DCM. Combined organic layer was dried over anhydrous Na₂SO₄ and concentrated under reduced pressure. The crude compound was purified by silica gel column chromatography (4% MeOH/DCM) to afford 17 (0.02 g, 34.5%) as an off white solid. ¹H NMR (400 MHz, DMSO-d₆) δ 12.63 (brs, 1H), 8.20 (s, 1H), 8.08 (d, J=7.83 Hz, 1H), 7.94 (s, 2H), 7.64 (d, J=6.85 Hz, 1H), 4.53-4.75 (m, 1H), 3.51-3.72 (m, 2H), 2.23 (brs, 1H), 2.02 (s, 3H), 1.96 (brs, 2H), 1.85 (s, 1H); LC-MS: m/z 374.15 [M+H]⁺; HPLC: 99.72%; SOR: 53.16, Solvent: DMSO, Path length: 100 mm, Concentration: 0.35 w/v %.

### Example 18. 1-acetyl-N-(7-fluoronaphtho[2,1-d]thiazol-2-yl)piperazine-2-carboxamide (18-a and 18-b)

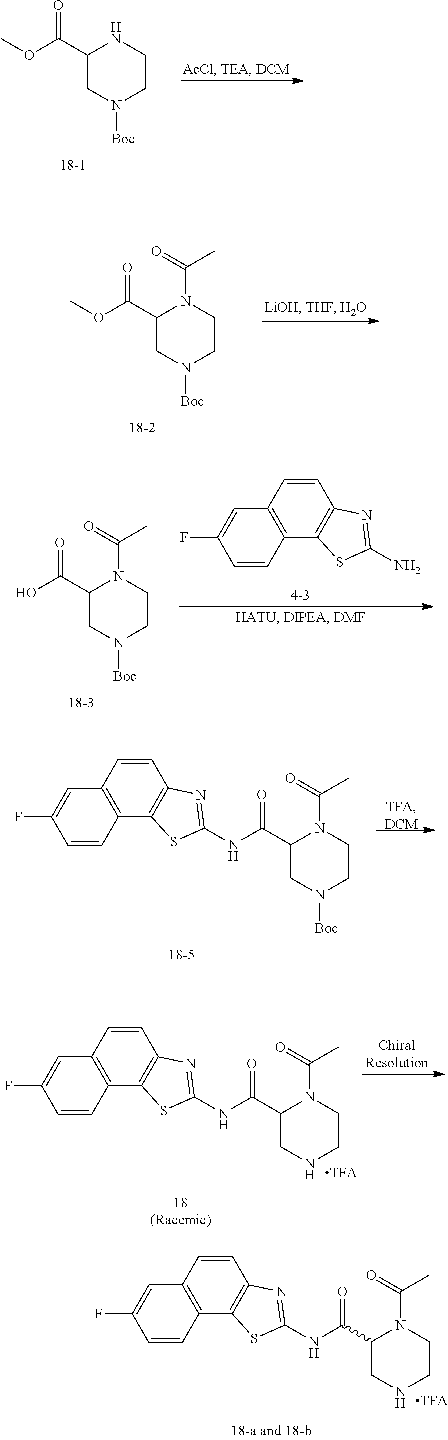

### 1-(tert-butyl) 3-methyl 4-acetylpiperazine-1,3-dicarboxylate (18-2)

To a stirred solution of 1-(tert-butyl) 3-methyl 4-acetylpiperazine-1,3-dicarboxylate (18-1, 2.0 g, 8.20 mmol) in DCM (20 mL), TEA (3.5 mL, 24.50 mmol) was added at RT and the reaction mixture was stirred for 10 min. To the resulting reaction mixture, acetyl chloride (0.7 mL, 10.20 mmol) was added at 0′C and the reaction mixture was stirred at RT for 2 h. The progress of the reaction was monitored by TLC. After completion of the reaction, the reaction mixture was poured into cold water. The aqueous layer was extracted with DCM. The organic layers were combined and dried over anhydrous Na2SO4 and concentrated under reduced pressure. The crude compound was purified by silica gel column chromatography (2% MeOH/DCM) to afford compound 18-2 (1.9 g, 82.5%) as colorless thick oil. 1H NMR (400 MHz, CHLOROFORM-d) δ 5.19 (d, J=2.93 Hz, 1H), 4.54-4.63 (m, 1H), 4.09 (brs, 1H), 3.74 (s, 3H), 3.48-3.66 (m, 2H), 3.01-3.12 (m, 1H), 2.88 (brs, 1H), 2.17 (s, 3H), 1.45 (s, 9H).

### 1-acetyl-4-(tert-butoxycarbonyl)piperazine-2-carboxylic acid (18-3)

To a stirred solution of compound 18-2 (1.0 g, 3.49 mmol) in THF (9 mL) and water (1 mL), LiOH.H2O (0.18 g, 4.19 mmol) was added and the reaction mixture was stirred at RT for 16 h. The progress of the reaction was monitored by TLC. After completion of the reaction, the reaction mixture was diluted with water and extracted with ethyl acetate. The aqueous layer was acidified with 2N HCl to pH 3. The aqueous layer was extracted with 10% MeOH/DCM. The organic layer were combined and dried over anhydrous Na2SO4 and concentrated under reduced pressure to afford compound 18-3 (0.8 g, 61.5%) as colorless thick oil which was used as such for the next reaction. 1H NMR (400 MHz, DMSO-d6) δ 12.90 (brs, 1H), 4.83 (d, J=3.42 Hz, 1H), 4.30-4.44 (m, 1H), 3.83 (d, J=5.38 Hz, 1H), 3.69 (d, J=13.21 Hz, 1H), 3.02-3.15 (m, 1H), 2.88 (brs, 1H), 2.66-2.80 (m, 1H), 2.04 (s, 3H), 1.38 (s, 9H); LC-MS: m/z 217.0 [M−56+H]+.

### tert-butyl 4-acetyl-3-((7-fluoronaphtho[2,1-d]thiazol-2-yl)carbamoyl)piperazine-1-carboxylate (18-5)

To a stirred solution of compound 3 (0.6 g, 2.21 mmol) in DMF (7 mL), DIPEA (1.2 mL, 6.62 mmol) was added followed by HATU (1.0 g, 2.65 mmol) at 0° C. and the reaction mixture was stirred at RT for 15 min. To the resulting reaction mixture, compound 4-3 (0.48 g, 2.21 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (70% EtOAc/hexane) to afford compound 18-5 (0.8 g, 80.0%) as an off white solid. LC-MS: m/z 473.20 [M+H]⁺.

### 1-acetyl-N-(7-fluoronaphtho[2,1-d]thiazol-2-yl)piperazine-2-carboxamide (18-a and 18-b)

A mixture of compound 18-5 (0.8 g, 1.70 mmol) and 30% TFA in DCM (15 mL) was stirred at 0° C. for 5 min followed by at RT for 3 h. The progress of the reaction was monitored by TLC. After completion of the reaction, the reaction mixture was concentrated under reduced pressure. The crude compound was triturated with diethyl ether followed by pentane and dried well to afford racemic 18 (Racemic)(TFA salt, 0.5 g) as a light brown solid. Chiral prep. HPLC purification of racemic 18 (Racemic) afforded 18-a (TFA salt, 0.155 g, 19.0%) and 18-b (TFA salt, 0.145 g, 18.0%) both as a light brown solid.

18-a: ¹H NMR (400 MHz, DMSO-d₆) δ 12.75 (brs, 1H), 9.14 (brs, 1H), 8.72 (brs, 1H), 8.08-8.22 (m, 1H), 7.88-8.02 (m, 3H), 7.52-7.61 (m, 1H), 5.44 (d, J=4.89 Hz, 1H), 3.99-4.07 (m, 2H), 3.90 (d, J=13.69 Hz, 2H), 3.28 (d, J=14.18 Hz, 1H), 3.10 (brs, 1H), 2.17 (s, 3H); LC-MS: m/z 372.20 [M+H]⁺; HPLC: 98.05%; C-HPLC: 100.00% (RT: 3.21); SOR: −18.18, Solvent: DMSO, Path length: 100 mm, Concentration: 0.255 w/v %.

18-b: ¹H NMR (400 MHz, DMSO-d₆) δ 12.70 (brs, 1H), 9.15 (brs, 1H), 8.71 (brs, 1H), 8.11-8.19 (m, 1H), 7.85-8.00 (m, 3H), 7.50-7.61 (m, 1H), 5.16-5.51 (m, 1H), 3.82-4.10 (m, 2H), 3.28 (d, J=12.72 Hz, 2H), 3.03-3.17 (m, 1H), 2.05-2.20 (m, 3H), 1H merged in solvent peak; LC-MS: m/z 373.15 [M+H]⁺; HPLC: 98.54%; C-HPLC: 99.15% (RT: 4.69); SOR: 9.70, Solvent: DMSO, Path length: 100 mm, Concentration: 0.255 w/v %.

### Example 19. (R)-4-acetyl-N-(7-fluoronaphtho[2,1-d]thiazol-2-yl)morpholine-3-carboxamide (19)

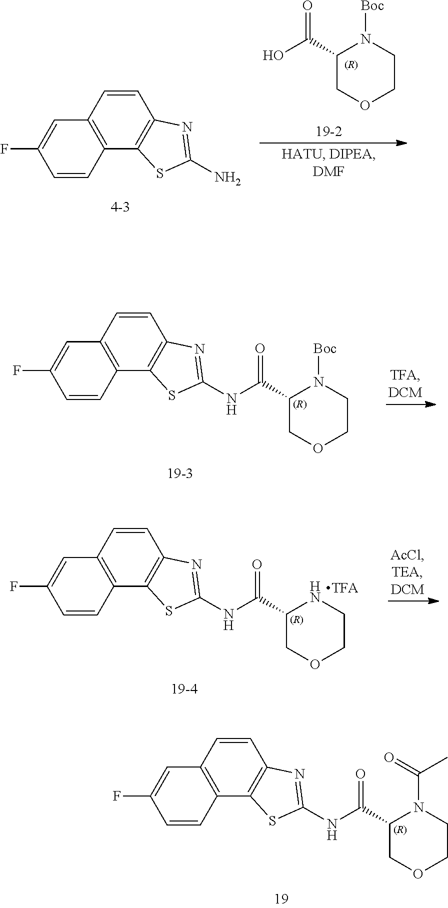

### tert-butyl (R)-3-((7-fluoronaphtho[2,1-d]thiazol-2-yl)carbamoyl)morpholine-4-carboxylate (19-3)

To a stirred solution of (R)-4-(tert-butoxycarbonyl)morpholine-3-carboxylic acid (19-2, 0.25 g, 1.10 mmol) in DMF (2 mL), was added DIPEA (0.36 g, 2.75 mmol) followed by HATU (0.42 g, 1.10 mmol) at 0° C. and the reaction mixture was stirred at RT for 10 min. To the resulting reaction mixture, compound 4-3 (0.20 g, 0.92 mmol) was added and the reaction mixture was stirred at RT for 12 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (25% EtOAc/hexane) to afford compound 19-3 (0.18 g, 45.5%) as an off white solid. LC-MS: m/z 431.8 [M+H]⁺.

### ((R)—N-(7-fluoronaphtho[2,1-d]thiazol-2-yl)morpholine-3-carboxamide TFA salt (19-4)

To a stirred solution of compound 19-3 (0.18 g, 0.41 mmol) in DCM (2 mL), TFA (1 mL) was added drop wise at 0° C. and the reaction mixture and then allowed to warm to RT for 2 h. The progress of the reaction was monitored by TLC. After completion of the reaction, the reaction mixture was concentrated under reduced pressure. The crude compound was triturated with diethyl ether followed by hexane and dried well to afford compound 19-4 (TFA salt, 0.19 g, crude) as an off white solid. ¹H NMR (400 MHz, DMSO-d₆) δ 13.20 (brs, 1H), 9.59 (brs, 2H), 8.19 (dd, J=5.38, 8.80 Hz, 1H), 7.89-8.02 (m, 3H), 7.53-7.61 (m, 1H), 4.45 (brs, 1H), 4.33 (dd, J=3.42, 12.23 Hz, 1H), 3.94 (d, J=12.72 Hz, 1H), 3.71-3.85 (m, 2H), 3.38 (d, J=13.21 Hz, 1H), 3.26 (brs, 1H); LC-MS: m/z 332.10 [M+H]⁺.

### (R)-4-acetyl-N-(7-fluoronaphtho[2,1-d]thiazol-2-yl)morpholine-3-carboxamide (19)

To a stirred solution of compound 19-4 (0.19 g, 0.43 mmol) in DCM (3 mL), TEA (0.13 g, 1.28 mmol) was added at RT and the reaction mixture was stirred for 15 min. To the resulting reaction mixture, acetyl chloride (0.05 g, 0.64 mmol) was added at 0° C. and the reaction mixture was stirred at RT for 2 h. The progress of the reaction was monitored by TLC. After completion of the reaction, the reaction mixture was diluted with ice cold water, aqueous layer was extracted with DCM. The organic layers were combined and dried over anhydrous Na2SO4 and concentrated under reduced pressure. The crude compound was purified by silica gel column chromatography (3% MeOH/DCM) followed by prep HPLC to afford 19 (0.035 g, 22.0%) as an off white solid. 1H NMR (400 MHz, DMSO-d₆) δ 12.75 (brs, 1H), 8.08-8.16 (m, 1H), 7.85-7.98 (m, 3H), 7.51-7.59 (m, 1H), 4.70-5.03 (m, 1H), 4.33-4.54 (m, 1H), 3.84-4.14 (m, 1H), 3.61-3.76 (m, 3H), 3.43-3.53 (m, 1H), 1.95-2.13 (m, 3H); LC-MS: m/z 374.15 [M+H]+; HPLC: 95.12%; SOR: 125.80, Solvent: Methanol, Path length: 100 mm. Concentration: 0.25 w/v %.

### Example 20. (S)-1-acetyl-N-(7-fluoronaphtho[2,1-d]thiazol-2-yl)piperidine-2-carboxamide (27)

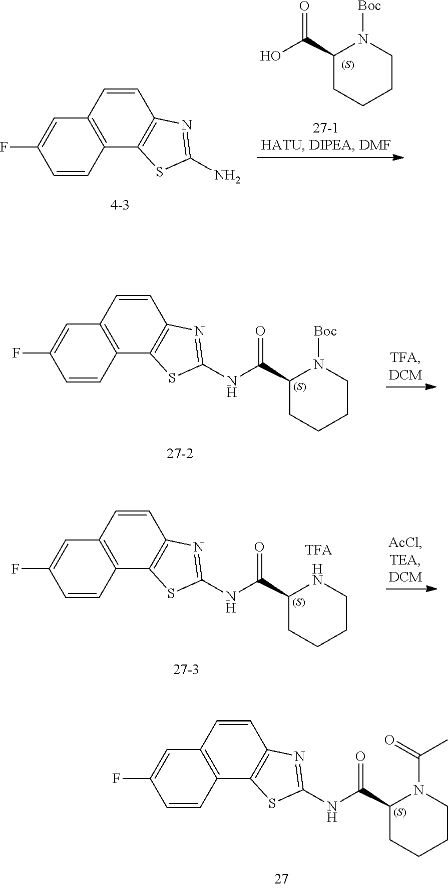

### Tert-butyl (S)-2-((7-fluoronaphtho[2,1-d]thiazol-2-yl)carbamoyl)piperidine-1-carboxylate (27-2)

To a stirred solution of (S)-1-(tert-butoxycarbonyl)piperidine-2-carboxylic acid (27-1, 0.32 g, 1.37 mmol) in DMF (2 mL), DIPEA (0.5 mL, 2.75 mmol) was added followed by HATU (0.52 g, 1.37 mmol) at 0° C. and the reaction mixture was stirred at RT for 15 min. To the resulting reaction mixture, compound 4-3 (0.20 g, 0.92 mmol) was added and the reaction mixture was stirred at 65° C. for 5 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was triturated with diethyl ether followed by hexane and dried well to afford 27-2 (0.145 g, 37.0%) as an off white solid. ¹H NMR (400 MHz, DMSO-d₆) δ 12.67 (s, 1H), 8.14 (dd, J=5.62, 9.05 Hz, 1H), 7.85-7.97 (m, 3H), 7.50-7.59 (m, 1H), 4.75-4.94 (m, 1H), 3.84 (d, J=12.23 Hz, 1H), 3.18-3.29 (m, 1H), 2.01-2.23 (m, 1H), 1.56-1.87 (m, 3H), 1.28-1.46 (m, 9H), 1.10-1.27 (m, 2H); LC-MS: m/z 428.15 [M−H]⁺; HPLC: 98.91%; SOR: −76.84, Solvent: Methanol, Path length: 100 mm, Concentration: 0.25 w/v %.

### (S)—N-(7-fluoronaphtho[2,1-d]thiazol-2-yl)piperidine-2-carboxamide (27-3)

A mixture of 27-2 (0.15 g, 0.34 mmol) and 30% TFA in DCM (7 mL) was stirred at 0° C. for 5 min followed by at RT for 4 h. The progress of the reaction was monitored by TLC. After completion of the reaction, the reaction mixture was concentrated under reduced pressure. The crude compound was triturated with diethyl ether followed by pentane and dried to afford 27-3 (TFA salt, 0.1 g) as an off white solid. ¹H NMR (400 MHz, DMSO-d₆) δ 9.10 (brs, 2H), 8.14-8.22 (m, 1H), 7.89-8.02 (m, 3H), 7.53-7.61 (m, 1H), 4.13 (d, J=9.29 Hz, 1H), 3.36-3.41 (m, 1H), 2.98-3.08 (m, 1H), 2.31 (d, J=10.27 Hz, 1H), 1.86 (d, J=11.74 Hz, 1H), 1.51-1.80 (m, 4H); LC-MS: m/z 330.0 [M+H]⁺; HPLC: 99.53%; SOR: 4.16, Solvent: Methanol, Path length: 100 mm, Concentration: 0.25 w/v %.

### (S)-1-acetyl-N-(7-fluoronaphtho[2,1-d]thiazol-2-yl)piperidine-2-carboxamide (27)

To a stirred solution of 27-3 (0.09 g, 0.27 mmol) in DCM (3 mL), TEA (0.1 mL, 0.82 mmol) was added at RT and the reaction mixture was stirred for 10 min. To the resulting reaction mixture, acetyl chloride (0.03 g, 0.32 mmol) was added at 0° C. and the reaction mixture was stirred at RT for 2 h. The progress of the reaction was monitored by TLC. After completion of the reaction, the reaction mixture was poured into cold water. The aqueous layer was extracted with DCM. The combined organic layers were dried over anhydrous Na₂SO₄ and concentrated under reduced pressure. The crude compound was purified by silica gel column chromatography (2% MeOH/DCM) to afford 27 (0.025 g, 25.5%) as an off white solid. ¹H NMR (400 MHz, DMSO-d₆) δ 12.60 (brs, 1H), 8.09-8.17 (m, 1H), 7.86-7.98 (m, 3H), 7.50-7.59 (m, 1H), 4.89-5.30 (m, 1H), 3.77-4.41 (m, 1H), 3.40-3.50 (m, 1H), 2.16-2.34 (m, 1H), 1.98-2.13 (m, 3H), 1.60-1.75 (m, 3H), 1.27-1.52 (m, 2H); LC-MS: m/z 371.8 [M+H]⁺; HPLC: 99.86%; SOR: −68.52, Solvent: Methanol, Path length: 100 mm, Concentration: 0.25 w/v %.

### Example 21. (1R,3S)-3-acrylamido-N-(7-fluoronaphtho[2,1-d]thiazol-2-yl)cyclohexane-1-carboxamide (33)

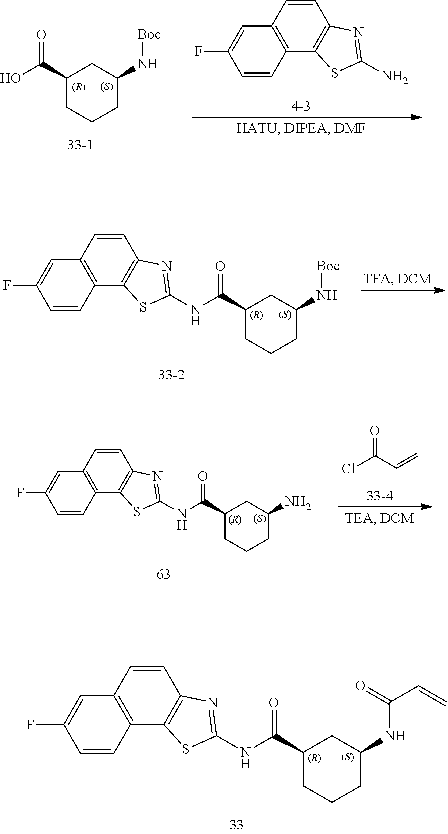

### Tert-butyl ((1S,3R)-3-((7-fluoronaphtho[2,1-d]thiazol-2-yl)carbamoyl)cyclohexyl)carbamate (33-2)

To a stirred solution of (1R,3S)-3-((tert-butoxycarbonyl)amino)cyclohexane-1-carboxylic acid (33-1, 0.17 g, 0.68 mmol) in DMF (5 mL) was added DIPEA (0.18 g, 1.37 mmol) followed by HATU (0.26 g, 0.68 mmol) at 0° C. and the reaction mixture was stirred at RT for 15 min. To the resulting reaction mixture compound 4-3 (0.10 g, 0.45 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (70% EtOAc/hexane) to afford compound 33-2 (0.14 g, 45.0%) as an off white solid. LC-MS: m/z 444.0 [M+H]⁺.

### (1R,3S)-3-amino-N-(7-fluoronaphtho[2,1-d]thiazol-2-yl)cyclohexane-1-carboxamide (63)

A mixture of compound 33-2 (0.14 g, 0.32 mmol) and 30% TFA in DCM (3 mL) was stirred at 0° C. for 5 min followed by at RT for 2 h. The progress of the reaction was monitored by TLC. After completion of the reaction the reaction mixture was concentrated under reduced pressure. The crude compound was triturated with diethyl ether followed by pentane and dried well to afford compound 63 (TFA salt, 0.14 g) as a light brown solid. LC-MS: m/z 344.0 [M+H]⁺.

### (1R,3S)-3-acrylamido-N-(7-fluoronaphtho[2,1-d]thiazol-2-yl)cyclohexane-1-carboxamide (33)

To a stirred solution of compound 63 (0.13 g, 0.37 mmol) in DCM (5 mL) was added TEA (0.11 g, 1.13 mmol) at RT and the reaction mixture was stirred for 10 min. To the resulting reaction mixture acryloyl chloride (33-4, 0.05 g, 0.56 mmol) was added at 0° C. and the reaction mixture was stirred at RT for 2 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by silica gel column chromatography (2% MeOH/DCM) followed by prep HPLC to afford 33 (0.013 g, 9.0%) as off white solid. ¹H NMR (400 MHz, DMSO-d₆) δ 12.48 (brs, 1H), 8.03-8.18 (m, 2H), 7.83-7.97 (m, 3H), 7.48-7.58 (m, 1H), 6.13-6.25 (m, 1H), 6.03-6.11 (m, 1H), 5.57 (d, J=9.78 Hz, 1H), 3.65-3.78 (m, 1H), 2.63-2.77 (m, 1H), 1.98-2.09 (m, 1H), 1.77-1.92 (m, 3H), 1.30-1.48 (m, 3H), 1.08-1.25 (m, 1H); LC-MS: m/z 397.9 [M+H]⁺; HPLC: 98.47%; SOR: −133.96, Solvent: DMSO, Path length: 100 mm, Concentration: 0.25 w/v %.

### Example 22. 1-acetyl-N-(7-fluoronaphtho[2,1-d]thiazol-2-yl)pyrrolidine-3-carboxamide (36), and 1-acetyl-N-(7-fluoronaphtho[2,1-d]thiazol-2-yl)pyrrolidine-3-carboxamide (82) and their Intermediates

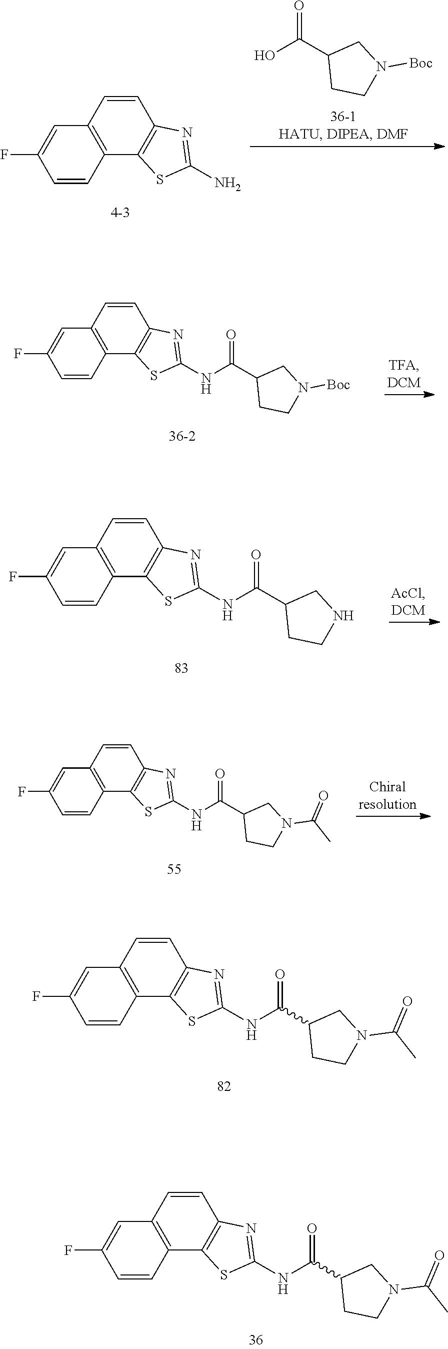

### Tert-butyl 3-((7-fluoronaphtho[2,1-d]thiazol-2-yl)carbamoyl)pyrrolidine-1-carboxylate (36-2)

To a stirred solution of 1-(tert-butoxycarbonyl)pyrolidine-3-carboxylic acid (36-1, 1.18 g, 5.50 mmol) in DMF (10 mL) at 0° C. was added DIPEA (2.5 mL, 13.76 mmol) followed by HATU (2.0 9 g, 5.50 mmol). The reaction mixture was stirred at RT for 15 min. To the resulting reaction mixture, compound 4-3 (1.00 g, 4.58 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (35% EtOAc/hexane) to afford compound 36-2 (1.13 g, 59.5%) as a brown solid. LC-MS: m/z 360.10 [M−56+H]⁺.

### N-(7-fluoronaphtho[2,1-d]thiazol-2-yl)pyrrolidine-3-carboxamide (83)

A mixture of compound 36-2 (1.00 g, 2.40 mmol) and 30% TFA in DCM (15 mL) was stirred at 0° C. for 5 min and then allowed to warm to RT and stirred at RT for 2 h. The progress of the reaction was monitored by TLC. After completion of the reaction the reaction mixture was concentrated under reduced pressure. The crude compound was triturated with diethyl ether followed by pentane and dried to afford 83 (TFA salt, 0.9 g) as an off white solid. ¹H NMR (400 MHz, DMSO-d₆) δ 12.80 (brs, 1H), 8.87-9.04 (m, 2H), 8.14 (dd, J=5.38, 8.80 Hz, 1H), 7.86-7.99 (m, 3H), 7.51-7.60 (m, 1H), 3.45-3.55 (m, 3H), 3.27 (t, J=7.09 Hz, 2H), 2.27-2.39 (m, 1H), 2.10-2.20 (m, 1H); LC-MS: m/z 316.0 [M+H]⁺; HPLC: 98.33%.

### 1-acetyl-N-(7-fluoronaphtho[2,1-d]thiazol-2-yl)pyrrolidine-3-carboxamide (55)

To a stirred solution of 83 (0.9 g, 2.09 mmol) in DCM (10 mL) was added TEA (0.9 mL, 6.20 mmol) and the reaction mixture was stirred for 10 min. The resulting reaction mixture was cooled to 0° C. and treated with acetyl chloride (0.24 mL, 3.10 mmol). The reaction mixture was stirred at RT for 2 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by silica gel column chromatography (3% MeOH/DCM) to afford 55 (0.6 g, 80.0%) as an off white solid. ¹H NMR (400 MHz, DMSO-d₆) δ 12.70 (brs, 1H), 8.14 (dd, J=5.62, 9.05 Hz, 1H), 7.85-7.97 (m, 3H), 7.51-7.59 (m, 1H), 3.55-3.80 (m, 2H), 3.41-3.54 (m, 2H), 2.14-2.34 (m, 2H), 2.02-2.12 (m, 1H), 1.97 (d, J=4.40 Hz, 3H); LC-MS: m/z 358.10 [M+H]⁺; HPLC: 99.15%.

Chiral prep. HPLC purification of racemic 55 afforded 82 (0.042 g) and 36 (0.028 g) both as an off white solid. (Compounds 82 and 36 are an enantiomeric pair whose absolute stereochemistry was not determined)

82: ¹H NMR (400 MHz, DMSO-d₆) δ 12.69 (brs, 1H), 8.11-8.16 (m, 1H), 7.86-7.97 (m, 3H), 7.51-7.58 (m, 1H), 3.56-3.79 (m, 2H), 3.41-3.55 (m, 2H), 2.01-2.35 (m, 3H), 1.96 (d, J=4.40 Hz, 3H); LC-MS: m/z 358.10 [M+H]⁺; HPLC: 98.35%; C-HPLC: 100.00% (RT: 9.88); SOR: 38.20, Solvent: DMSO, Path length: 100 mm, Concentration: 0.25 w/v %.

36: ¹H NMR (400 MHz, DMSO-d₆) δ 12.60 (brs, 1H), 8.14 (dd, J=5.62, 9.05 Hz, 1H), 7.86-7.97 (m, 3H), 7.51-7.58 (m, 1H), 3.55-3.80 (m, 2H), 3.40-3.55 (m, 2H), 2.01-2.37 (m, 3H), 1.96 (d, J=4.40 Hz, 3H); LC-MS: m/z 358.10 [M+H]⁺; HPLC: 98.92%; C-HPLC: 99.98% (RT: 16.26); SOR: −44.59, Solvent: DMSO, Path length: 100 mm, Concentration: 0.25 w/v %.

Chiral Prep. HPLC Method:

Column: PHENOMENEX CELLULOSE-4, 250 mm*30 mm, 5 u
Mobile Phase: A: n-HEXANE+0.1% TFA

B: Ethanol

Flow rate: 30 mL/min

### Isocratic: 15% B

### Example 23. 1-acetyl-N-(7-fluoronaphtho[2,1-d]thiazol-2-yl)piperidine-3-carboxamide (38), (45)

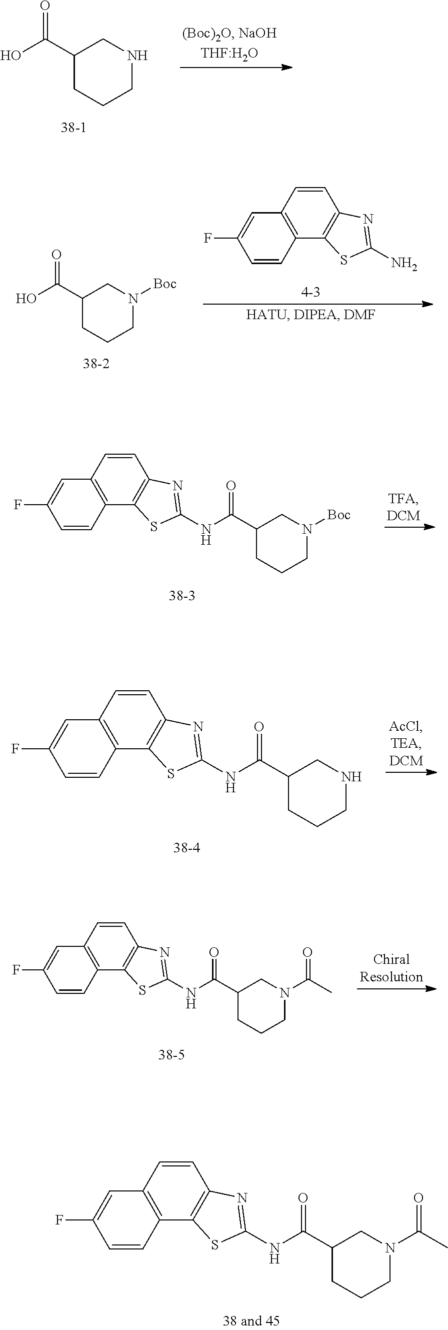

### 1-(Tert-butoxycarbonyl)piperidine-3-carboxylic acid (2)

To a stirred solution of piperidine-3-carboxylic acid (38-1, 1.0 g, 7.74 mmol) in THF:H₂O (20 mL) were added NaOH (0.46 g, 11.61 mmol) and (Boc)₂O (1.9 g, 8.51 mmol) at room temperature. The mixture was stirred at rt for 12 h. The progress of the reaction was monitored by TLC and LCMS. After completion of the reaction the reaction mixture was quenched with water and extracted with ethyl acetate (3×30 mL). The combined organic layers were washed with brine, dried over anhydrous Na₂SO₄, filtered and concentrated under reduced pressure resulting in the crude compound. The crude compound was purified by 100-200 mesh silica gel column chromatography (20% EtOAc/hexane) to afford the title compound 38-2 (1.2 g, 67.5%) as an off-white solid. LC-MS: m/z 230.0 [M+H]⁺.

### Tert-butyl 3-((7-fluoronaphtho[2,1-d]thiazol-2-yl)carbamoyl)piperidine-1-carboxylate (38-3)

To a stirred solution of compound 38-2 (1.2 g, 5.23 mmol) in DMF (10 mL) was added DIPEA (2.9 mL, 15.69 mmol) followed by HATU (3.0 g, 7.85 mmol) at 0° C. and the reaction mixture was stirred at RT for 15 min. To the resulting reaction mixture compound 4-3 (1.14 g, 5.23 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (50% EtOAc/hexane) to afford compound 38-3 (1.0 g, 44.5%) as an off white solid. LC-MS: m/z 430.0 [M+H]⁺.

### N-(7-fluoronaphtho[2,1-d]thiazol-2-yl)piperidine-3-carboxamide (38-4)

A mixture of compound 38-3 (1.0 g, 2.33 mmol) and 30% TFA in DCM (15 mL) was stirred at 0° C. for 5 min followed by RT for 3 h. The progress of the reaction was monitored by TLC. After completion of the reaction the reaction mixture was concentrated under reduced pressure. The crude compound was triturated with diethyl ether followed by pentane and dried well to afford compound 38-4 (TFA salt, 1.0 g) as a light brown solid. LC-MS: m/z 330.0 [M+H]⁺.

### 1-acetyl-N-(7-fluoronaphtho[2,1-d]thiazol-2-yl)piperidine-3-carboxamide (38-5)

To a stirred solution of compound 38-4 (1.0 g, 3.04 mmol) in DCM (20 mL), was added TEA (1.3 mL, 9.10 mmol) at RT and the reaction mixture was stirred for 10 min. To the resulting reaction mixture acetyl chloride (0.32 mL, 4.56 mmol) was added at 0° C. and the reaction mixture was stirred at RT for 2 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by silica gel column chromatography (2% MeOH/DCM) to afford compound 38-5 (0.3 g, 27.0%) as an off white solid.

Chiral prep. HPLC purification of racemic compound 38-5 afforded 38 (0.01 g, 3.33%) and 45 (0.01 g, 3.33%) both as an off white solid.

Chiral Prep. HPLC Method:
Column: DIACEL CHIRALPAK-IG, 250 mm*30 mm, 5 u
Mobile Phase: A: n-HEXANE+0.1% Iso-propyl-amine

B: DCM:MeOH(90:10)

Flow rate: 28 mL/min

### Isocratic: 40% B

38:

¹H NMR (400 MHz, DMSO-d₆) δ 12.63 (brs, 1H), 8.10-8.19 (m, 1H), 7.85-7.98 (m, 3H), 7.54 (t, J=7.82 Hz, 1H), 4.11-4.47 (m, 1H), 3.72-4.02 (m, 1H), 3.04-3.21 (m, 1H), 2.59-2.93 (m, 2H), 2.05 (d, J=16.63 Hz, 3H), 1.65-1.84 (m, 2H), 1.27-1.53 (m, 1H), 1.17 (d, J=6.36 Hz, 1H); LC-MS: m/z 372.0) [M+H]⁺; HPLC: 99.54%; C-HPLC: 100.00)% (RT: 12.03).

45:

¹H NMR (400 MHz, DMSO-d₆) δ 12.63 (brs, 1H), 8.08-8.18 (m, 1H), 7.85-7.99 (m, 3H), 7.50-7.59 (m, 1H), 4.13-4.47 (m, 1H), 3.72-4.01 (m, 1H), 3.04-3.22 (m, 1H), 2.71-2.92 (m, 1H), 2.60-2.70 (m, 1H), 2.05 (d, J=16.63 Hz, 3H), 1.65-1.84 (m, 2H), 1.24-1.52 (m, 1H), 1.17 (d, J=6.36 Hz, 1H); LC-MS: m/z 372.00 [M+H]⁺; HPLC: 99.45%; C-HPLC: 99.11% (RT: 16.46).

### Example 24. (1S,3R)-3-acrylamido-N-(7-fluoronaphtho[2,1-d]thiazol-2-yl)cyclohexane-1-carboxamide (41)

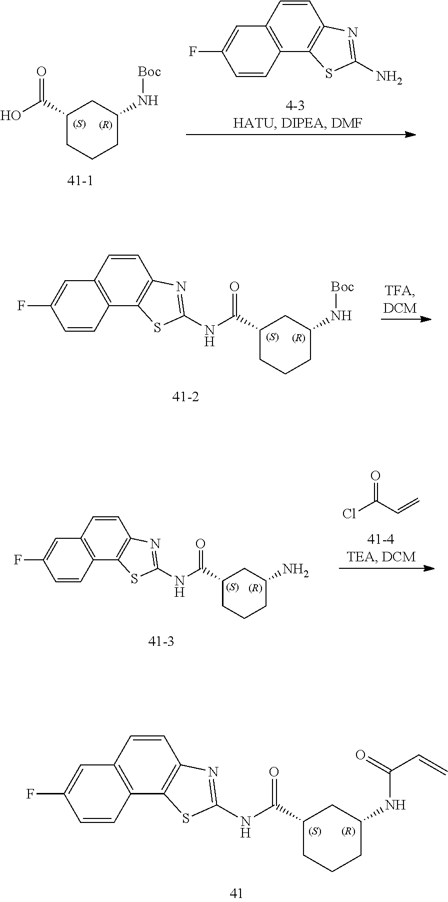

### Tert-butyl ((1R,3S)-3-((7-fluoronaphtho[2,1-d]thiazol-2-yl)carbamoyl)cyclohexyl)carbamate (41-2)

To a stirred solution of (1S,3R)-3-((tert-butoxycarbonyl)amino)cyclohexane-1-carboxylic acid (41-1, 0.50 g, 2.06 mmol) in DMF (5 mL) at 0° C. was added DIPEA (0.76 mL, 4.13 mmol) followed by HATU (0.79 g, 2.06 mmol). The reaction mixture was stirred at RT for 15 min. To the resulting reaction mixture was added compound 4-3 (0.30 g, 1.38 mmol) and the reaction mixture was stirred at RT for 14 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (30% EtOAc/hexane) to afford compound 41-2 (0.5 g, 55.0%) as an off white solid. ¹H NMR (400 MHz, DMSO-d₆) δ 12.47 (s, 1H), 8.12 (dd, J=5.38, 8.80 Hz, 1H), 7.85-7.95 (m, 3H), 7.49-7.57 (m, 1H), 6.86 (d, J=7.83 Hz, 1H), 2.64 (brs, 1H), 1.94-2.02 (m, 1H), 1.74-1.88 (m, 3H), 1.38 (s, 9H), 1.26-1.35 (m, 3H), 1.07-1.24 (m, 2H); LC-MS: m/z 444.31 [M+H]⁺.

### (1S,3R)-3-amino-N-(7-fluoronaphtho[2,1-d]thiazol-2-yl)cyclohexane-1-carboxamide (41-3)

A mixture of compound 41-2 (0.50 g, 1.13 mmol) and 30% TFA in DCM (9 mL) was stirred at 0° C. for 5 min and then allowed to warm to RT and stirred at RT for 2 h. The progress of the reaction was monitored by TLC. After completion of the reaction, the reaction mixture was concentrated under reduced pressure. The crude compound was triturated with diethyl ether followed by pentane and dried to afford compound 41-3 (TFA salt, 0.3 g) as an off white solid. LC-MS: m/z 344.27 [M+H]⁺.

### (1S,3R)-3-acrylamido-N-(7-fluoronaphtho[2,1-d]thiazol-2-yl)cyclohexane-1-carboxamide (41)

To a stirred solution of compound 41-3 (0.20 g, 0.58 mmol) in DCM (10 mL) was added TEA (0.25 mL, 1.74 mmol) at RT and the reaction mixture was stirred for 10 min. The resulting reaction mixture was cooled to 0° C. and treated with acryloyl chloride (5, 0.08 g, 0.87 mmol). The reaction mixture was stirred at RT for 3 h. The progress of the reaction was monitored by TLC. After completion of the reaction, the reaction mixture was poured into cold water. The aqueous layer was extracted with DCM. The organic layer was dried over anhydrous Na₂SO₄ and concentrated under reduced pressure. The crude compound was purified by silica gel column chromatography (3% MeOH/DCM) to afford 41 (0.005 g, 2.0%) as an off white solid. ¹H NMR (400 MHz, DMSO-d₆) δ 12.50 (brs, 1H), 8.06-8.16 (m, 2H), 7.85-7.95 (m, 3H), 7.50-7.57 (m, 1H), 6.15-6.25 (m, 1H), 6.04-6.12 (m, 1H), 5.57 (dd, J=2.20, 10.03 Hz, 1H), 3.64-3.75 (m, 1H), 2.64-2.77 (m, 1H), 2.02 (d, J=11.74 Hz, 1H), 1.77-1.93 (m, 3H), 1.32-1.46 (m, 3H), 1.12-1.23 (m, 1H); LC-MS: m/z 398.25 [M+H]⁺; HPLC: 98.79%.

### Example 25. (R)-1-acryloyl-N-(7-fluoronaphtho[2,1-d]thiazol-2-yl)pyrrolidine-2-carboxamide (44)

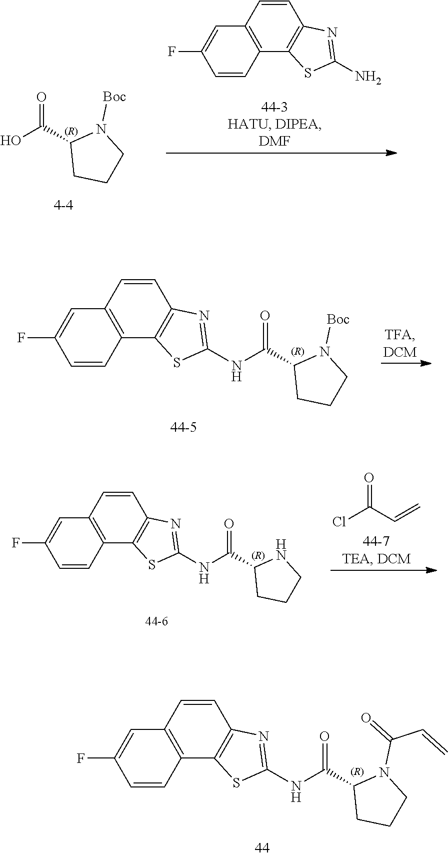

### Tert-butyl (R)-2-((7-fluoronaphtho[2,1-d]thiazol-2-yl)carbamoyl)pyrrolidine-1-carboxylate (4-5)

To a stirred solution of (tert-butoxycarbonyl)-D-proline (44-4, 0.32 g, 1.40 mmol) in DMF (10 mL) at 0° C. was added DIPEA (0.39 mL, 2.90 mmol) followed by HATU (0.57 g, 1.49 mmol). The reaction mixture was stirred at RT for 15 min. To the resulting reaction mixture compound 4-3 (0.20 g, 0.90 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (5% MeOH/DCM) to afford compound 44-5 (0.20 g, 48.5%) as an off white solid. ¹H NMR (400 MHz, DMSO-d₆) δ 12.63-12.76 (m, 1H), 8.10-8.19 (m, 1H), 7.86-7.97 (m, 3H), 7.54 (t, J=8.80 Hz, 1H), 4.38-4.51 (m, 1H), 3.43-3.52 (m, 1H), 3.33-3.42 (m, 1H), 2.22-2.35 (m, 1H), 1.76-2.01 (m, 3H), 1.41 (s, 3H), 1.24 (s, 6H); LC-MS: m/z 416.26 [M+H]⁺.

### (R)—N-(7-fluoronaphtho[2,1-d]thiazol-2-yl)pyrrolidine-2-carboxamide (4-6)

A mixture of compound 44-5 (0.20 g, 0.48 mmol) and 30% TFA in DCM (12 mL) was stirred at 0° C. for 5 min and then allowed to warm to RT and stirred at RT for 3 h. The progress of the reaction was monitored by TLC. After completion of the reaction, the reaction mixture was concentrated under reduced pressure. The crude compound was triturated with diethyl ether followed by pentane and dried well to afford compound 44-6 (TFA salt, 0.10 g) as an off white solid.

### (R)-1-acryloyl-N-(7-fluoronaphtho[2,1-d]thiazol-2-yl)pyrrolidine-2-carboxamide (44)

To a stirred solution of compound 44-6 (0.10 g, 0.31 mmol) in DCM (10 mL) was added TEA (0.13 mL, 0.95 mmol). The reaction mixture was stirred for 10 min. The resulting reaction mixture was cooled to 0° C. and treated with acryloyl chloride (44-7, 0.03 mL, 0.41 mmol). The reaction mixture was stirred at RT for 2 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by silica gel column chromatography (5% MeOH/DCM) to afford 44 (0.0075 g, 6.5%) as an off white solid. ¹H NMR (400 MHz, DMSO-d₆) δ 12.64 (brs, 1H), 8.08-8.15 (m, 1H), 7.86-7.96 (m, 3H), 7.50-7.59 (m, 1H), 6.67 (dd, J=10.52, 16.87 Hz, 1H), 6.11-6.19 (m, 1H), 5.74 (dd, J=1.96, 10.76 Hz, 1H), 4.64-4.70 (m, 1H), 3.65-3.82 (m, 2H), 2.20-2.30 (m, 1H), 1.92-2.07 (m, 3H); LC-MS: m/z 370.20 [M+H]⁺; HPLC: 97.90%; SOR: 234.91, Solvent: MeOH, Path length: 100 mm, Concentration: 0.25 w/v %.

### Example 26. (S)-1-acetyl-N-(7-fluoronaphtho[2,1-d]thiazol-2-yl)piperidine-3-carboxamide (45) and tert-butyl (S)-3-((7-fluoronaphtho[2,1-d]thiazol-2-yl)carbamoyl)piperidine-1-carboxylate (62)

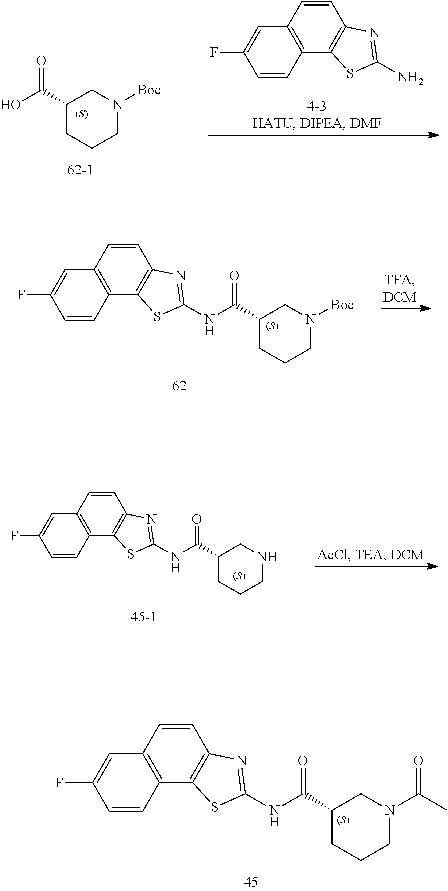

### Tert-butyl (S)-3-((7-fluoronaphtho[2,1-d]thiazol-2-yl)carbamoyl)piperidine-1-carboxylate (62)

To a stirred solution of (S)-1-(tert-butoxycarbonyl)piperidine-3-carboxylic acid (62-1, 0.25 g, 1.10 mmol) in DMF (2 mL) at 0° C. was added DIPEA (0.5 mL, 2.75 mmol) followed by HATU (0.52 g, 1.37 mmol) and the reaction mixture was stirred at RT for 15 min. To the resulting reaction mixture compound 4-3 (0.20 g, 0.92 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (50% EtOAc/hexane) to afford 62 (0.12 g, 30.5%) as an off white solid. ¹H NMR (400 MHz, DMSO-d₆) δ 12.60 (brs, 1H), 8.11-8.16 (m, 1H), 7.85-7.97 (m, 3H), 7.50-7.58 (m, 1H), 3.63-4.17 (m, 2H), 2.86-3.03 (m, 1H), 2.62-2.79 (m, 1H), 1.92-2.08 (m, 1H), 1.60-1.86 (m, 2H), 1.37 (s, 9H), 2H merged in solvent peak; LC-MS: m/z 430.25 [M+H]⁺; HPLC: 99.91%; SOR: 113.64, Solvent: Methanol, Path length: 100 mm, Concentration: 0.235 w/v %.

### (S)—N-(7-fluoronaphtho[2,1-d]thiazol-2-yl)piperidine-3-carboxamide (45-1)

A mixture of 62 (0.12 g, 0.28 mmol) and 30% TFA in DCM (3 mL) was stirred at 0° C. for 5 min and then allowed to warm to RT and stirred at RT for 4 h. The progress of the reaction was monitored by TLC. After completion of the reaction, the reaction mixture was concentrated under reduced pressure. The crude compound was triturated with diethyl ether followed by pentane and dried to afford 45-1 (TFA salt, 0.1 g) as a brown solid. LC-MS: m/z 329.69 [M+H]⁺.

### (S)-1-acetyl-N-(7-fluoronaphtho[2,1-d]thiazol-2-yl)piperidine-3-carboxamide (45)

To a stirred solution of 45-1 (0.1 g, 0.30 mmol) in DCM (2 mL) was added TEA (0.1 mL, 0.91 mmol) at RT and the reaction mixture was stirred for 10 min. The resulting reaction mixture was cooled to 0° C. and treated with acetyl chloride (0.04 g, 0.46 mmol). The reaction mixture was stirred at RT for 2 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by silica gel column chromatography (2% MeOH/DCM) to afford 45 (0.0104 g, 10.0%) as an off white solid. ¹H NMR (400 MHz, DMSO-d₆) δ 12.64 (brs, 1H), 8.14 (brs, 1H), 7.85-7.98 (m, 3H), 7.54 (t, J=7.83 Hz, 1H), 4.12-4.47 (m, 1H), 3.70-4.01 (m, 1H), 2.82-3.15 (m, 1H), 2.61-2.80 (m, 2H), 2.05 (d, J=17.12 Hz, 3H), 1.64-1.83 (m, 2H), 1.27-1.53 (m, 1H), 1H merged in solvent peak; LC-MS: m/z 372.85 [M+H1]; HPLC: 98.13%; SOR: 96.73, Solvent: Methanol, Path length: 100 mm, Concentration: 0.235 w/v %.

### Example 27. (S)—N-(7-fluoronaphtho[2,1-d]thiazol-2-yl)pyrrolidine-2-carboxamide (47)

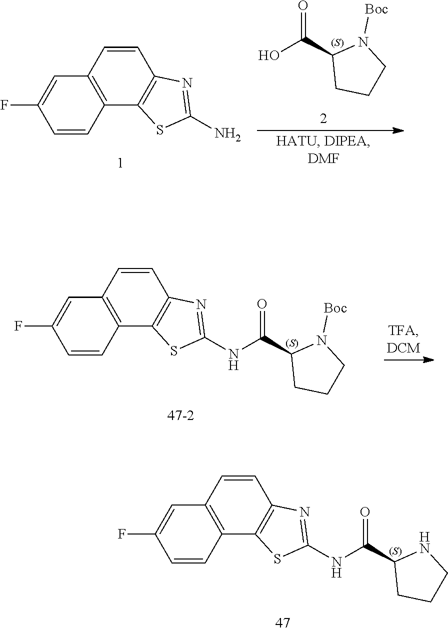

### Tert-butyl (S)-2-((7-fluoronaphtho[2,1-d]thiazol-2-yl)carbamoyl)pyrrolidine-1-carboxylate (47-2)

To a stirred solution of (tert-butoxycarbonyl)-D-proline (47-1, 0.44 g, 2.1 mmol) in DMF (3 mL), DIPEA (0.53 g, 4.11 mmol) was added followed by HATU (0.78 g, 2.1 mmol) at 0° C. and the reaction mixture was stirred at RT for 15 min. To the resulting reaction mixture, compound 4-3 (0.30 g, 1.37 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (70% EtOAc/hexane) to afford compound 47-2 (0.4 g, 70.0%) as an off white solid. LC-MS: m/z 416.0 [M+H]⁺.

### (S)—N-(7-fluoronaphtho[2,1-d]thiazol-2-yl)pyrrolidine-2-carboxamide (47)

A mixture of compound 47-2 (0.38 g, 0.92 mmol) and 30% TFA in DCM (8 mL) was stirred at 0° C. for 5 min followed by RT for 3 h. The progress of the reaction was monitored by TLC. After completion of the reaction, the reaction mixture was concentrated under reduced pressure. The crude compound was purified by trituration with diethyl ether and pentane followed by SFC purification to afford 47 (TFA salt, 0.015 g) as an off white solid. ¹H NMR (400 MHz, DMSO-d₆) δ 8.13-8.20 (m, 1H), 7.93-8.01 (m, 2H), 7.88-7.93 (m, 1H), 7.53-7.61 (m, 1H), 4.47-4.55 (m, 1H), 3.25-3.30 (m, 2H), 2.37-2.46 (m, 1H), 2.02-2.14 (m, 1H), 1.90-2.00 (m, 2H), 2H merged in solvent peak; LC-MS: m/z 316.2 [M+H]⁺; HPLC: 99.64%.

### Example 28. N-(7-fluoronaphtho[2,1-d]thiazol-2-yl)isoxazole-5-carboxamide (49)

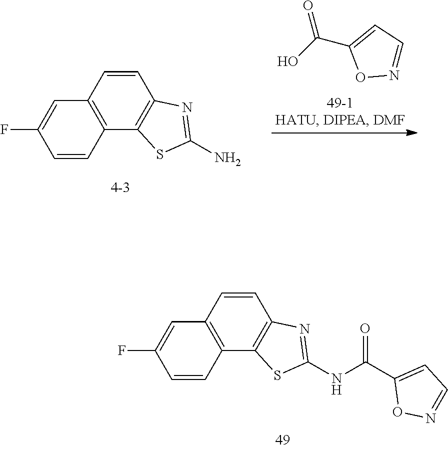 [SMILES: Nc1nc2ccc3cc(F)ccc3c2s1 ; O=C(O)c1ccno1 ; O=C(Nc1nc2ccc3cc(F)ccc3c2s1)c1ccno1]

### N-(7-fluoronaphtho[2,1-d]thiazol-2-yl)isoxazole-5-carboxamide (49)

To a stirred solution of isoxazole-5-carboxylic acid 49-1 (0.16 g, 1.37 mmol) in DMF (3 mL) at 0° C. was added DIPEA (0.5 mL, 2.75 mmol) followed by HATU (0.52 g, 1.37 mmol). The reaction mixture was stirred at RT for 15 min. To the resulting reaction mixture compound 1 (0.20 g, 0.92 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (5% MeOH/DCM) to afford 49 (0.011 g, 4.0%) as an off white solid. ¹H NMR (400 MHz, DMSO-d₆) δ 13.62 (brs, 1H), 8.89 (s, 1H), 8.17-8.28 (m, 1H), 7.87-8.05 (m, 3H), 7.50-7.63 (m, 2H); LC-MS: m/z 314.05 [M+H]⁺; HPLC: 99.65%:

### Example 29. (N-(7-fluoronaphtho[2,1-d]thiazol-2-yl)-1H-pyrazole-4-carboxamide (50)

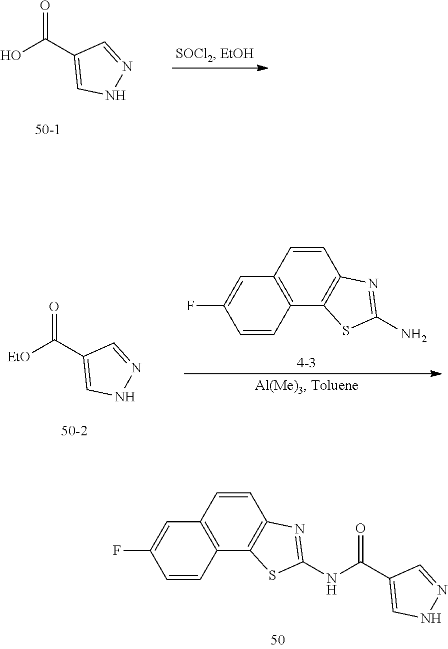 [SMILES: O=C(O)c1cn[nH]c1 ; CCOC(=O)c1cn[nH]c1 ; Nc1nc2ccc3cc(F)ccc3c2s1 ; O=C(Nc1nc2ccc3cc(F)ccc3c2s1)c1cn[nH]c1]

### ethyl 1H-pyrazole-4-carboxylate (50-2)

To a solution of 1H-pyrazole-4-carboxylic acid (50-1, 1 g, 8.92 mmol) in EtOH (10 mL) was added thionyl chloride (1.6 g, 13.38 mmol) at 0° C. and the mixture was stirred at rt for 3 h. The progress of the reaction was monitored by TLC. After completion of the reaction, all the volatiles were evaporated to dryness. The mixture was diluted with water and extracted with EtOH/DCM (10%). The combined organic layers were washed with water and brine, dried over anhydrous Na₂SO₄ and concentrated under reduced pressure. The crude compound was purified by silica gel column chromatography (15% EtOAc/hexane) to afford compound 50-2 (1.0 g, 80.0%) as an off white solid. LC-MS: m/z 141.0 [M+H]⁺.

### N-(7-fluoronaphtho[2,1-d]thiazol-2-yl)-1H-pyrazole-4-carboxamide (50)

To a solution of compound 50-2 (0.07 g, 0.50 mmol) in toluene (10 mL) were added compound 4-3 (0.10 g, 0.46 mmol) and Al(Me)₃ (1.14 mL, 2.29 mmol). The reaction mixture was stirred 100° C. for 12 h. The progress of the reaction was monitored by TLC. After completion of the reaction, all the volatiles were evaporated and the residue was diluted with water and extracted with ethyl acetate (5×50 mL). The combined organic layers were washed with brine, dried over Na₂SO₄, filtered and concentrated under reduced pressure. The crude compound was purified over 100-200 mesh neutral silica gel column chromatography (40% ethyl acetate/n-hexane) to afford the title 50 (0.01 g, 6.5%) as an off white solid. ¹H NMR (400 MHz, DMSO-d₆) δ 13.50 (brs, 1H), 12.71 (brs, 1H), 8.63 (brs, 1H), 8.21-8.32 (m, 1H), 8.16 (dd, J=5.62, 9.05 Hz, 1H), 7.85-8.00 (m, 3H), 7.51-7.60 (m, 1H); LC-MS: m/z 313.15 [M+H]⁺; HPLC: 96.74%.

### Example 30. N-(7-fluoronaphtho[2,1-d]thiazol-2-yl)pyrrolidine-3-carboxamide (51) and N-(7-fluoronaphtho[2,1-d]thiazol-2-yl)pyrrolidine-3-carboxamide (37)

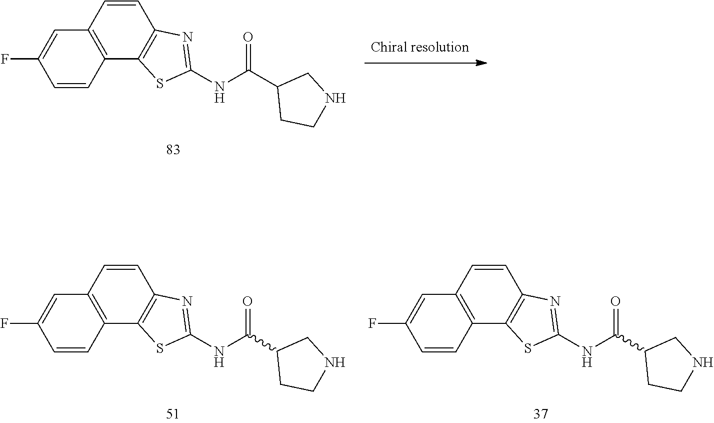 [SMILES: O=C(Nc1nc2ccc3cc(F)ccc3c2s1)C1CCNC1 ; O=C(Nc1nc2ccc3cc(F)ccc3c2s1)C1CCNC1 ; O=C(Nc1nc2ccc3cc(F)ccc3c2s1)C1CCNC1]

Chiral prep. HPLC purification of racemic 83 afforded 51 (0.0055 g) and 37 (0.0012 g) both as an off white solid. (Compounds 51 and 37 are an enantiomeric pair whose absolute stereochemistry was not determined)

51: ¹H NMR (400 MHz, DMSO-d₆) δ 8.11 (dd, J=5.87, 8.80 Hz, 1H), 7.83-7.95 (m, 3H), 7.49-7.58 (m, 1H), 3.17 (brs, 2H), 2.83-3.05 (m, 3H), 1.90-2.07 (m, 2H), 2H merged in solvent peak; LC-MS: m/z 315.9 [M+H]⁺; HPLC: 97.717%; C-HPLC: 100.00% (RT: 8.82).

Chiral Prep. HPLC Method:

Column: DIACEL CHIRALPAK-IG, 250 mm*30 mm, 5 u
Mobile Phase: A: MTBE+0.1% Iso-propyl-amine

B: DCM:MeOH(90:10)

Flow rate: 30 mL/min

### Isocratic: 70% B

37: ¹H NMR (400 MHz, DMSO-d₆) δ 8.11 (dd, J=5.87, 8.80 Hz, 1H), 7.84-7.95 (m, 3H), 7.71-7.79 (m, 1H), 7.49-7.64 (m, 1H), 3.13 (d, J=6.36 Hz, 2H), 2.80-3.00 (m, 3H), 1.89-2.03 (m, 2H), 1H merged in solvent peak; LC-MS: m/z 316.2 [M+H]⁺; HPLC: 98.17%; C-HPLC: 98.83% (RT: 12.17).

### Example 31. (S)-1-acetyl-N-(7-fluoronaphtho[2,1-d]thiazol-2-yl)pyrrolidine-3-carboxamide (52)

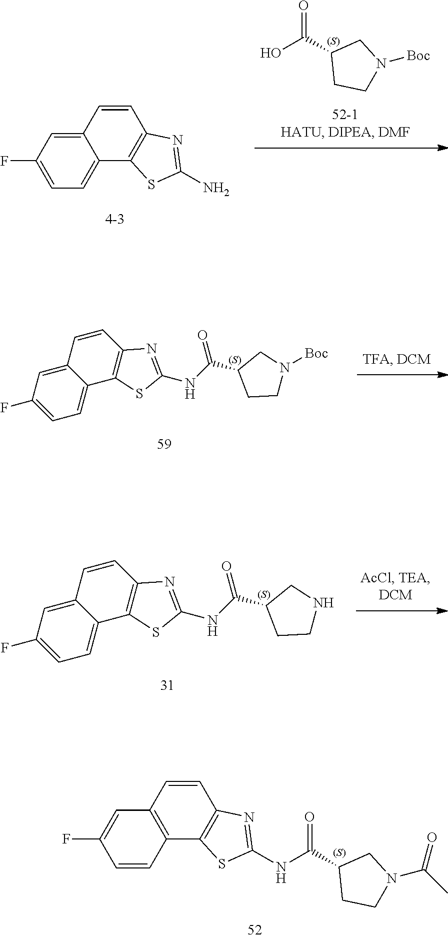

### Tert-butyl (S)-3-((7-fluoronaphtho[2,1-d]thiazol-2-yl)carbamoyl)pyrrolidine-1-carboxylate (59)

To a stirred solution of (S)-1-(tert-butoxycarbonyl)pyrrolidine-3-carboxylic acid (52-1, 0.49 g, 2.29 mmol) in DMF (5 mL) was cooled to 0° C. DIPEA (0.59 mL, 3.44 mmol) was added followed by HATU (0.65 g, 1.72 mmol) and the reaction mixture was stirred at RT for 15 min. To the resulting reaction mixture compound 4-3 (0.25 g, 1.14 mmol was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (30% EtOAc/hexane) to afford 59 (0.35 g, 79.0%) as a light brown solid. ¹H NMR (400 MHz, DMSO-d₆) δ 12.61-12.77 (m, 1H), 8.08-8.20 (m, 1H), 7.83-7.99 (m, 3H), 7.48-7.59 (m, 1H), 4.37-4.51 (m, 1H), 3.43-3.54 (m, 1H), 3.34-3.42 (m, 1H), 2.16-2.36 (m, 1H), 1.77-2.01 (m, 3H), 1.40 (s, 3H), 1.24 (s, 6H); LC-MS: m/z 416.0 [M+H]⁺; HPLC: 99.64%; C-HPLC: 100.00% (RT: 7.10); SOR: 61.94, Solvent: DMSO, Path length: 100 mm, Concentration: 0.25 w/v %.

### (S)—N-(7-fluoronaphtho[2,1-d]thiazol-2-yl)pyrrolidine-3-carboxamide (31)

A mixture of 52-2 (0.33 g, 0.79 mmol) and 30% TFA in DCM (6 mL) was stirred at 0° C. for 5 min and then allowed to warm to RT and stirred at RT for 16 h. The progress of the reaction was monitored by TLC. After completion of the reaction the reaction mixture was concentrated under reduced pressure. The crude compound was triturated with diethyl ether followed by pentane and dried well to afford 31 (TFA salt, 0.28 g) as a light brown solid. ¹H NMR (400 MHz, DMSO-d₆) δ 9.50 (brs, 1H), 8.13-8.22 (m, 1H), 7.95-8.03 (m, 2H), 7.92 (d, J=9.78 Hz, 1H), 7.53-7.61 (m, 1H), 4.55 (t, J=7.58 Hz, 1H), 2.38-2.47 (m, 1H), 2.03-2.14 (m, 1H), 1.92-2.02 (m, 2H), 1.05-1.13 (m, 1H), 2H merged in solvent peak; LC-MS: m/z 315.76 [M+H]⁺; HPLC: 99.72%; C-HPLC: 100.00% (RT: 13.18); SOR: 45.00, Solvent: DMSO, Path length: 100 mm, Concentration: 0.25 w/v %.

### (S)-1-acetyl-N-(7-fluoronaphtho[2,1-d]thiazol-2-yl)pyrrolidine-3-carboxamide (52)

To a stirred solution of 52-3 (0.15 g, 0.36 mmol) in DCM (10 mL) was added TEA (0.15 mL, 1.09 mmol) at RT and the reaction mixture was stirred for 10 min. The resulting reaction mixture was cooled to 0° C. and treated with acetyl chloride (0.03 mL, 0.54 mmol). The reaction mixture was stirred at RT for 3 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by silica gel column chromatography (2% MeOH/DCM) to afford 52 (0.09 g, 69.0%) as an off white solid. ¹H NMR (400 MHz, DMSO-d₆) δ 12.49-12.61 (m, 1H), 8.07-8.17 (m, 1H), 7.84-7.98 (m, 3H), 7.55 (t, J=8.80 Hz, 1H), 4.53-4.78 (m, 1H), 3.50-3.74 (m, 2H), 2.22 (brs, 1H), 2.03 (s, 3H), 1.82-1.99 (m, 3H); LC-MS: m/z 357.9 [M+H]⁺; HPLC: 99.78%; C-HPLC: 99.89% (RT: 7.57); SOR: 58.04, Solvent: DMSO, Path length: 100 mm, Concentration: 0.25 w/v %.

### Example 32. N-(7-fluoronaphtho[2,1-d]thiazol-2-yl)-1H-imidazole-4-carboxamide (53)

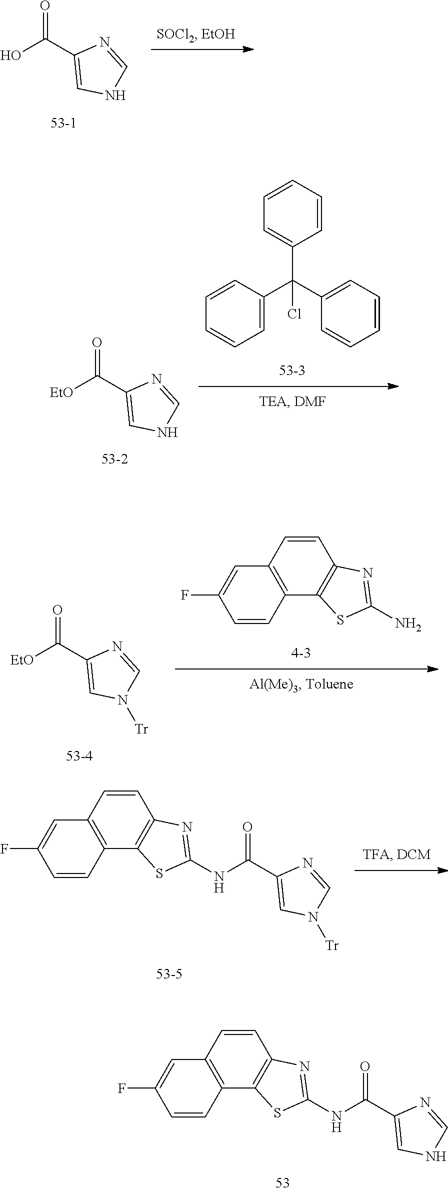

### Ethyl 1H-imidazole-4-carboxylate (53-2)

To a solution of 1H-imidazole-4-carboxylic acid (53-1, 1 g, 8.92 mmol) in EtOH (10 mL) was added thionyl chloride (1.6 g, 13.38 mmol) at 0° C. and the mixture was stirred at RT for 3 h. The progress of the reaction was monitored by TLC. After completion of the reaction, all the volatiles were evaporated to dryness and the residue was diluted with water. The mixture was extracted with EtOH/DCM (10%). The combined organic layers were washed with water and brine, dried over anhydrous Na₂SO₄ and concentrated under reduced pressure. The crude compound was purified by silica gel column chromatography (15% EtOAc/hexane) to afford pure compound 53-2 (1.0 g, 80.0%) as an off white solid. LC-MS: m/z 141.0 [M+H]⁺.

### Ethyl 1-trityl-1H-imidazole-4-carboxylate (53-4)

To a stirred solution of compound 53-2 (0.20 g, 1.43 mmol) in DMF (10 mL) were added TEA (0.6 mL, 4.29 mmol) and (chloromethanetriyl)tribenzene (53-3, 0.80 g, 2.86 mmol) at RT. The mixture was stirred at RT for 1 h. The progress of the reaction was monitored by TLC and LCMS. After completion of the reaction, the reaction mixture was quenched with water and extracted with DCM (3×30 mL). The combined organic layers were washed with brine, dried over anhydrous Na₂SO₄, filtered and concentrated under reduced pressure resulting in the crude compound. The crude compound was purified by 100-200 mesh silica gel column chromatography (0.5% MeOH/DCM) to afford the title compound 53-4 (0.29 g, 53.0%) as brown sticky solid. ¹H NMR (400 MHz, DMSO-d₆) δ 7.94-7.96 (m, 1H), 7.56 (d, J=0.98 Hz, 1H), 7.39-7.46 (m, 9H), 7.11 (dd, J=1.47, 7.83 Hz, 6H), 4.19 (q, J=6.85 Hz, 2H), 1.22 (t, J=7.09 Hz, 3H).

### N-(7-fluoronaphtho[2,1-d]thiazol-2-yl)-1-trityl-1H-imidazole-4-carboxamide (53-5)

To a solution of compound 53-4 (0.19 g, 0.50 mmol) in toluene (8 mL) were added compound 4-3 (0.10 g, 0.46 mmol) and Al(Me)₃ (1.14 mL, 2.29 mmol) and the reaction mixture was stirred at 100° C. for 12 h. The progress of the reaction was monitored by TLC. After completion of the reaction, all the volatiles were evaporated and the residue was diluted with water. The mixture was extracted with ethyl acetate (5×50 mL). The combined organic layers were washed with brine, dried over Na₂SO₄, filtered and concentrated under reduced pressure. The crude compound was purified over 100-200 mesh neutral silica gel column chromatography (10% ethyl acetate/n-hexane) to afford the compound 53-5 (0.07 g, 27.5%) as an off white sticky solid. LC-MS: m/z 553.35 [M−H]⁺.

### N-(7-fluoronaphtho[2,1-d]thiazol-2-yl)-1H-imidazole-4-carboxamide (53)

A mixture of compound 53-5 (0.07 g, 0.13 mmol) and 30% TFA in DCM (3.5 mL) was stirred at 0° C. for 5 min followed by at RT for 3 h. The progress of the reaction was monitored by TLC. After completion of the reaction, the reaction mixture was concentrated under reduced pressure. The crude compound was triturated with diethyl ether followed by pentane and dried well to afford 53 (TFA salt, 0.024 g, 61.5%) as an off white solid. ¹H NMR (400 MHz, DMSO-d) δ 12.60 (brs, 1H), 8.35 (brs, 1H), 8.28 (brs, 1H), 8.19 (dd, J=5.38, 8.80 Hz, 1H), 7.93-8.01 (m, 2H), 7.86-7.92 (m, 1H), 7.51-7.60 (m, 1H), 1H merged in solvent peak; LC-MS: m/z 313.15 [M+H]⁺; HPLC: 98.18%.

### Example 33. (1S,3R)—N-(7-fluoronaphtho[2,1-d]thiazol-2-yl)-3-morpholinocyclopentane-1-carboxamide (57)

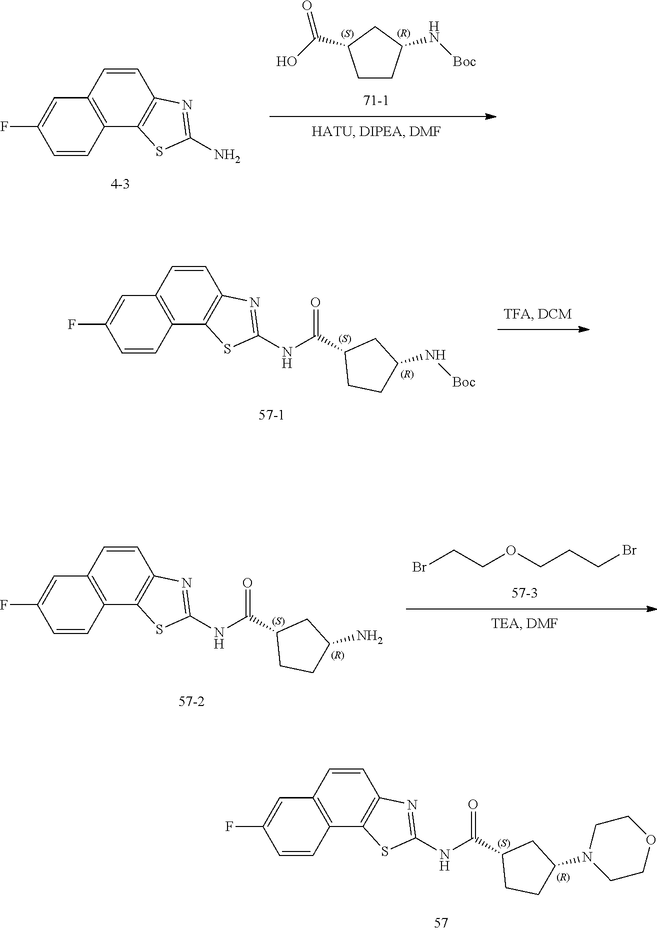

### tert-butyl ((1R,3S)-3-((7-fluoronaphtho[2,1-d]thiazol-2-yl)carbamoyl)cyclopentyl)carbamate (57-1)

To a stirred solution of (1S,3R)-3-((tert-butoxycarbonyl)amino)cyclopentane-1-carboxylic acid (71-1, 0.5 g, 2.18 mmol) in DMF (8 mL) at 0° C. was added DIPEA (1.2 mL, 6.54 mmol) followed by HATU (1.24 g, 3.27 mmol) and the reaction mixture was stirred at RT for 15 min. To the resulting reaction mixture compound 4-3 (0.48 g, 2.18 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (50% EtOAc/hexane) to afford compound 57-1 (0.8 g, 85.5%) as an off white solid. LC-MS: m/z 430.0 [M+H]⁺.

### (1S,3R)-3-amino-N-(7-fluoronaphtho[2,1-d]thiazol-2-yl)cyclopentane-1-carboxamide (57-2)

A mixture of compound 57-1 (0.8 g, 1.86 mmol) and 30% TFA in DCM (10 mL) was stirred at 0° C. for 5 min followed by at RT for 3 h. The progress of the reaction was monitored by TLC. After completion of the reaction the reaction mixture was concentrated under reduced pressure. The crude compound was triturated with diethyl ether followed by pentane and dried well to afford compound 57-2 (TFA salt, 0.5 g) as a light brown solid. LC-MS: m/z 330.0 [M+H]⁺.

### (1S,3R)—N-(7-fluoronaphtho[2,1-d]thiazol-2-yl)-3-morpholinocyclopentane-1-carboxamide (57)

To a stirred solution of compound 57-2 (0.30 g, 0.91 mmol) in DMF (5 mL) was added TEA (0.19 mL, 2.74 mmol) at RT followed by compound 57-3 (0.32 g, 1.37 mmol) and the reaction mixture was stirred at 80° C. for 15 h. The progress of the reaction was monitored by TLC. After completion of the reaction the reaction was quenched with saturated NH₄Cl solution. The aqueous layer was extracted with ethyl acetate. The organic layer was washed with brine, dried over anhydrous Na₂SO₄ and concentrated under reduced pressure to afford 57 (0.012 g, 3.0%) as an off white solid. ¹H NMR (400 MHz, DMSO-d6) δ 12.60 (brs, 1H), 8.13 (dd, J=5.38, 9.29 Hz, 1H), 7.84-7.96 (m, 3H), 7.49-7.57 (m, 1H), 3.60 (t, J=4.40 Hz, 4H), 2.99-3.10 (m, 1H), 2.55-2.64 (m, 1H), 2.43 (brs, 4H), 2.09-2.21 (m, 1H), 1.75-1.97 (m, 3H), 1.53-1.75 (m, 2H); LC-MS: m/z 400.30 [M+H]⁺; HPLC: 97.88%; C-HPLC: 99.90% (RT: 4.59); SOR: 33.62, Solvent: DMSO, Path length: 100 mm, Concentration: 0.25 w/v %.

### Example 34. N-(7-fluoronaphtho[2,1-d]thiazol-2-yl)azetidine-3-carboxamide (58) and 1-acetyl-N-(7-fluoronaphtho[2,1-d]thiazol-2-yl)azetidine-3-carboxamide (28)

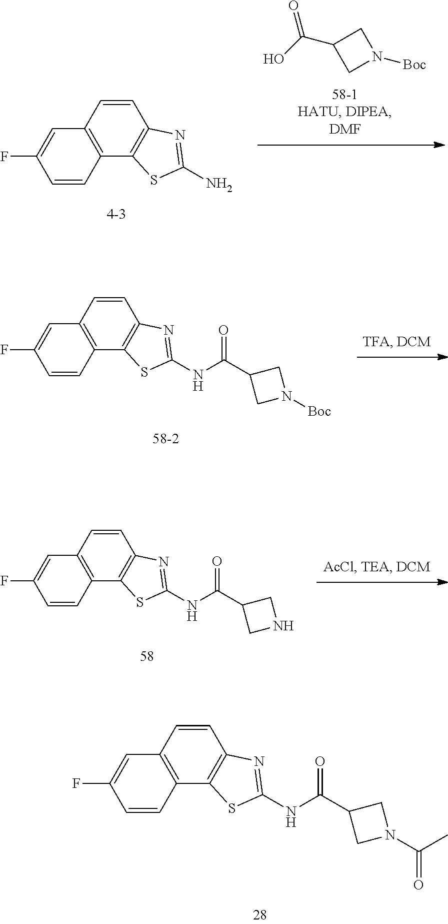 [SMILES: CC(C)(C)OC(=O)N1CC(C(=O)O)C1 ; Nc1nc2ccc3cc(F)ccc3c2s1 ; CC(C)(C)OC(=O)N1CC(C(=O)Nc2nc3ccc4cc(F)ccc4c3s2)C1 ; O=C(Nc1nc2ccc3cc(F)ccc3c2s1)C1CNC1 ; CC(=O)N1CC(C(=O)Nc2nc3ccc4cc(F)ccc4c3s2)C1]

### Tert-butyl 3-((7-fluoronaphtho[2,1-d]thiazol-2-yl)carbamoyl)azetidine-1-carboxylate (3)

To a stirred solution of 1-(tert-butoxycarbonyl)azetidine-3-carboxylic acid (58-1, 0.15 g, 0.75 mmol) in DMF (5 mL) at 0° C. was added DIPEA (0.4 mL, 2.24 mmol) followed by HATU (0.43 g, 1.12 mmol). The reaction mixture was stirred at RT for 15 min. To the resulting reaction mixture, compound 4-3 (0.33 g, 1.49 mmol) was added and the reaction mixture was stirred at RT for 14 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (50% EtOAc/hexane) to afford compound 58-2 (0.13 g, 43.5%) as an off white solid. LC-MS: m/z 402.12 [M+H]⁺.

### N-(7-fluoronaphtho[2,1-d]thiazol-2-yl)azetidine-3-carboxamide (58)

A mixture of compound 58-2 (0.13 g, 0.32 mmol) and 30% TFA in DCM (5 mL) was stirred at 0° C. for 5 min and then allowed to warm to RT and stirred at RT for 1 h. The progress of the reaction was monitored by TLC. After completion of the reaction the reaction mixture was concentrated under reduced pressure. The crude compound was triturated with diethyl ether followed by pentane and dried well to afford 58 (TFA salt, 0.11 g) as an off white solid. ¹H NMR (400 MHz, DMSO-d₆) δ 8.13-8.22 (m, 1H), 7.88-8.00 (m, 3H), 7.56 (t, J=8.07 Hz, 1H), 4.10-4.24 (m, 4H), 3.92-4.01 (m, 1H), 2H merged in solvent peak; LC-MS: m/z 302.3 [M+H]⁺; HPLC: 98.69%.

### 1-Acetyl-N-(7-fluoronaphtho[2,1-d]thiazol-2-yl)azetidine-3-carboxamide (28)

To a stirred solution of 28 (0.05 g, 0.17 mmol) in DCM (4 mL) was added TEA (0.05 g, 0.50 mmol) and the reaction mixture was stirred for 10 min. The resulting reaction mixture was cooled to 0° C. and treated with acetyl chloride (0.02 g, 0.25 mmol) and the reaction mixture was stirred at RT for 1 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by silica gel column chromatography (2% MeOH/DCM) to afford 28 (0.011 g, 19.0%) as an off white solid. ¹H NMR (400 MHz, DMSO-d₆) δ 12.60 (brs, 1H), 8.12-8.19 (m, 1H), 7.85-7.96 (m, 3H), 7.50-7.58 (m, 1H), 4.26-4.38 (m, 2H), 3.97-4.07 (m, 2H), 3.67-3.78 (m, 1H), 1.78 (s, 3H); LC-MS: m/z 343.9 [M+H]⁺; HPLC: 98.86%.

### Example 35. (R)—N-(7-fluoronaphtho[2,1-d]thiazol-2-yl)tetrahydrofuran-3-carboxamide (60) and (S)—N-(7-fluoronaphtho[2,1-d]thiazol-2-yl)tetrahydrofuran-3-carboxamide (91)

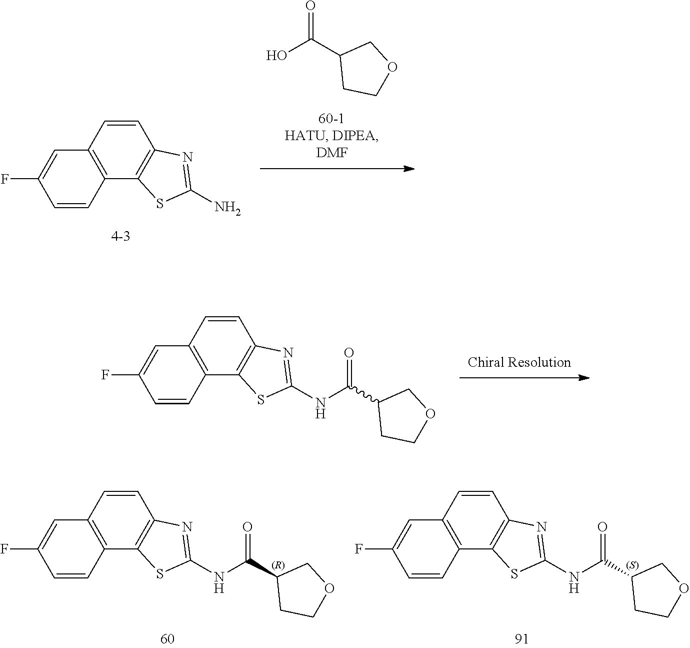

### N-(7-fluoronaphtho[2,1-d]thiazol-2-yl)tetrahydrofuran-3-carboxamide

To a stirred solution of tetrahydrofuran-3-carboxylic acid (60-1, 0.32 g, 2.75 mmol) in DMF (5 mL) at 0° C. was added DIPEA (1.3 mL, 6.88 mmol) followed by HATU (1.0 g, 2.75 mmol) and the reaction mixture was stirred at RT for 15 min. To the resulting reaction mixture, compound 4-3 (0.50 g, 2.29 mmol was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (25% EtOAc/hexane) to afford a mixture of 60 and 91 (0.3 g, 41.5%) as light brown solid.

Chiral prep. HPLC purification of racemic Mixture of 60 and 91 afforded 60 (0.10 g, 33.0%) and 91 (0.10 g, 33.0%) both as light brown solid.

Chiral Prep. HPLC Method:

Column: DIACEL CHIRALPAK-IA, 250 mm*30 mm, 5 u
Mobile Phase: A: n-Hexane+0.1% TFA

B: Iso-propyl-alcohol

Flow rate: 28 mL/min

### Isocratic: 35% B

60: ¹H NMR (400 MHz, DMSO-d₆) δ 12.63 (brs, 1H), 8.10-8.17 (m, 1H), 7.86-7.97 (m, 3H), 7.51-7.58 (m, 1H), 3.93-3.99 (m, 1H), 3.79-3.86 (m, 2H), 3.69-3.77 (m, 1H), 3.34-3.42 (m, 1H), 2.11-2.20 (m, 2H); LC-MS: m/z 316.95 [M+H]⁺; HPLC: 98.03%; C-HPLC: 99.74% (RT: 8.28); SOR: −20.44, Solvent: DMSO, Path length: 100 mm, Concentration: 0.25 w/v %.

91: ¹H NMR (400 MHz, DMSO-d₆) δ 12.63 (brs, 1H), 8.14 (dd, J=5.38, 8.80 Hz, 1H), 7.83-7.97 (m, 3H), 7.50-7.59 (m, 1H), 3.94-4.01 (m, 1H), 3.79-3.86 (m, 2H) 3.73 (q, J=7.66 Hz, 1H), 3.34-3.44 (m, 1H), 2.09-2.22 (m, 2H); LC-MS: m/z 317.00 [M+H]⁺; HPLC: 98.25%; C-HPLC: 99.12% (RT: 6.25); SOR: 14.28, Solvent: DMSO, Path length: 100 mm, Concentration: 0.25 w/v %.

### Example 36. 1-acetyl-N-(7-fluoronaphtho[2,1-d]thiazol-2-yl)-4-methylpiperazine-2-carboxamide (66, 32)

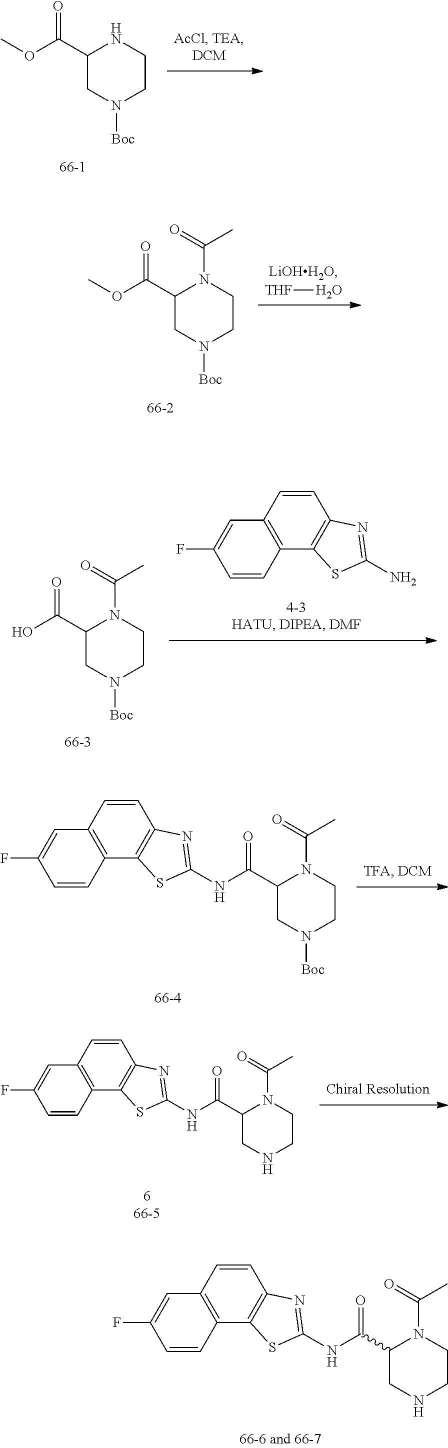

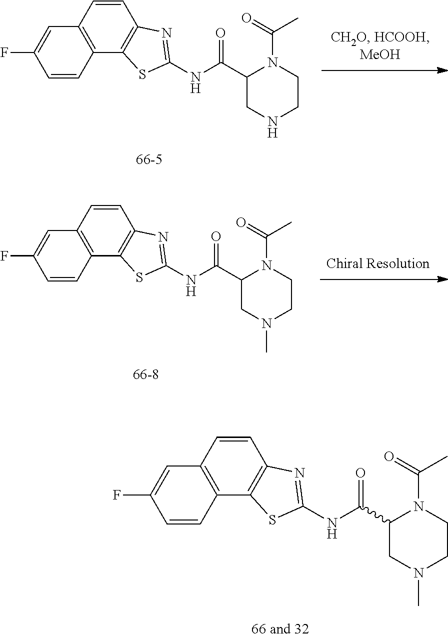 [SMILES: CC(=O)N1CCNCC1C(=O)Nc1nc2ccc3cc(F)ccc3c2s1 ; CC(=O)N1CCN(C)CC1C(=O)Nc1nc2ccc3cc(F)ccc3c2s1 ; CC(=O)N1CCN(C)CC1C(=O)Nc1nc2ccc3cc(F)ccc3c2s1]

### 1-(tert-butyl) 3-methyl 4-acetylpiperazine-1,3-dicarboxylate (66-2)

To a stirred solution of I-(tert-butyl) 3-methyl piperazine-1,3-dicarboxylate (66-1, 2.0 g, 8.20 mmol) in DCM (20 mL), TEA (3.5 mL, 24.50 mmol) was added at RT and the reaction mixture was stirred for 10 min. To the resulting reaction mixture, acetyl chloride (0.7 mL, 10.20 mmol) was added at 0° C. and the reaction mixture was stirred at RT for 2 h. The progress of the reaction was monitored by TLC. After completion of the reaction, the reaction mixture was poured into cold water. The aqueous layer was extracted with DCM. The combined organic layers were dried over anhydrous Na₂SO₄ and concentrated under reduced pressure. The crude compound was purified by silica gel column chromatography (2% MeOH/DCM) to afford compound 66-2 (1.9 g, 82.5%) as colorless thick oil. ¹H NMR (400 MHz, CDCl₃) δ 5.19 (d, J=2.93 Hz, 1H), 4.53-4.63 (m, 1H), 4.09 (brs, 1H), 3.70-3.81 (m, 3H), 3.46-3.67 (m, 2H), 2.99-3.15 (m, 1H), 2.79-2.97 (m, 1H), 2.04-2.22 (m, 3H), 1.45 (s, 9H).

### 1-acetyl-4-(tert-butoxycarbonyl)piperazine-2-carboxylic acid (66-3)

To a stirred solution of compound 66-2 (1.9 g, 6.64 mmol) in THF (18 mL) and water (2 mL). LiOH.H₂O (0.84 g, 19.90 mmol) was added and the reaction mixture was stirred at RT for 4 h. The progress of the reaction was monitored by TLC. After completion of the reaction, the reaction mixture was diluted with water and extracted with ethyl acetate. The aqueous layer was acidified with 1N HC to pH 3. The aqueous layer was extracted with 10% MeOH/DCM. The combined organic layers were dried over anhydrous Na₂SO₄ and concentrated under reduced pressure to afford compound 66-3 (1.5 g, crude) as an off white solid. LC-MS: m/z 271.0 [M−H]⁺.

### Tert-butyl 4-acetyl-3-((7-fluoronaphtho[2,1-d]thiazol-2-yl)carbamoyl)piperazine-1-carboxylate (66-4)

To a stirred solution of compound 66-3 (1.4 g, 5.10 mmol) in DMF (10 mL), DIPEA (2.82 mL, 15.00 mmol) was added followed by HATU (2.32 g, 6.10 mmol) at 0° C. and the reaction mixture was stirred at RT for 15 min. To the resulting reaction mixture, compound 4-3 (1.12 g, 5.10 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (80% EtOAc/hexane) to afford compound 66-4 (1.0 g, 41.0%) as a dark brown solid. LC-MS: m/z 473.25 [M+H]⁺.

### 1-acetyl-N-(7-fluoronaphtho[2,1-d]thiazol-2-yl)piperazine-2-carboxamide (66-5)

A mixture of compound 66-4 (1.0 g, 2.10 mmol) and 30% TFA in DCM (25 mL) was stirred at 0° C. for 10 min followed by RT for 4 h. The progress of the reaction was monitored by TLC. After completion of the reaction, the reaction mixture was concentrated under reduced pressure. The crude compound was triturated with diethyl ether followed by pentane and dried well to afford compound 66-5 (TFA salt, 1.0 g) as a light brown solid. LC-MS: m/z 373.15 [M+H]⁺.

Chiral prep. HPLC purification of racemic 66-5 (0.5 g) afforded 66-6 (0.055 g, 11.0%) and 66-7 (0.040 g, 8.0%) both as a light brown solid.

Chiral Prep. HPLC Method:

Column: Chiralpak IG 250 mm×30 mm 5μ
Mobile phase A: CO₂
Mobile phase B: 0.1% NH₃ in Methanol
Gradient T/% of B: 30 to 50% 5 min, 50 to 50% 9 min, 50 to 30% 5 min.
Flow rate 80 mL/min.

66-6: ¹H NMR (400 MHz, DMSO-d₆) δ 12.75 (brs, 1H), 9.12 (brs, 1H), 8.71 (brs, 1H), 8.12-8.19 (m, 1H), 7.88-8.01 (m, 3H), 7.57 (t, J=7.58 Hz, 1H), 5.44 (d, J=4.89 Hz, 1H), 4.03 (d, J=13.69 Hz, 2H), 3.90 (d, J=13.69 Hz, 2H), 3.21-3.32 (m, 2H), 2.17 (s, 3H); LC-MS: m/z 373.20 [M+H]⁺; HPLC: 98.05%; C-HPLC: 100.00% (RT: 3.21); SOR: −18.18, Solvent: DMSO, Path length: 100 mm, Concentration: 0.255 w/v %.

66-7: ¹H NMR (400 MHz, DMSO-d₆) δ 12.75 (brs, 1H), 9.15 (brs, 1H), 8.71 (brs, 1H), 8.12-8.20 (m, 1H), 7.87-8.01 (m, 3H), 7.53-7.62 (m, 1H), 5.16-5.47 (m, 1H), 3.85-4.07 (m, 2H), 3.28 (d, J=12.72 Hz, 2H), 2.92-3.17 (m, 2H), 2.08-2.20 (m, 3H); LC-MS: m/z 373.15 [M+H]⁺; HPLC: 98.54%; C-HPLC: 99.15% (RT: 4.69); SOR: 9.70, Solvent: DMSO, Path length: 100 mm, Concentration: 0.255 w/v %.

### 1-acetyl-N-(7-fluoronaphtho[2,1-d]thiazol-2-yl)-4-methylpiperazine-2-carboxamide (66 and 32)

To a stirred solution of racemic 66-5 (0.5 g, 1.30 mmol) in MeOH (5 mL), formaldehyde (7 mL, 37% solution in water) was added and the mixture was stirred for 10 min followed by the addition of formic acid (7 mL). The reaction mixture was stirred at 90° C. for 16 h. The progress of the reaction was monitored by TLC. After completion of the reaction, all volatiles were evaporated and the crude compound was purified by silica gel column chromatography (4% MeOH/DCM) to afford the title compound 66-8 (0.25 g, 48.0%) as a light brown thick liquid.

Chiral prep. HPLC purification of racemic 66-8 afforded 66 (0.005 g, 2.0%) and 32 (0.005 g, 2.0%) both as an off white solid.

Chiral Prep. HPLC Method:

Column: DIACEL CHIRALPAK-IC, 250 mm*30 mm, 5 u
Mobile Phase: A: n-Hexane+0.1% Iso-propyl-amine

B: Ethanol

Flow rate: 25 mL/min

### Isocratic: 30% B

66: ¹H NMR (400 MHz, DMSO-d₆) δ 12.62 (brs, 1H), 8.09-8.17 (m, 1H), 7.86-7.97 (m, 3H), 7.51-7.59 (m, 1H), 5.13-5.19 (m, 1H), 3.75 (d, J=12.72 Hz, 1H), 3.59 (dd, J=9.29, 12.23 Hz, 1H), 2.73-2.81 (m, 1H), 2.17 (brs, 3H), 2.11 (s, 3H), 1.14-1.25 (m, 3H); LC-MS: m/z 387.2 [M+H]⁺; HPLC: 99.88%; C-HPLC: 100.00% (RT: 10.84).

32: ¹H NMR (400 MHz, DMSO-d₆) δ 12.63 (brs, 1H), 8.08-8.19 (m, 1H), 7.87-7.99 (m, 3H), 7.52-7.59 (m, 1H), 5.15 (brs, 1H), 3.69-3.81 (m, 1H), 3.52-3.64 (m, 1H), 2.71-2.84 (m, 1H), 2.17 (brs, 3H), 2.11 (s, 3H), 1.14-1.26 (m, 3H); LC-MS: m/z 387.0 [M+H]⁺; HPLC: 99.71%; C-HPLC: 96.26% (RT: 15.36).

### Example 37. (R)-1-acetyl-N-(7-fluoronaphtho[2,1-d]thiazol-2-yl)azetidine-2-carboxamide (69) and Its Intermediates

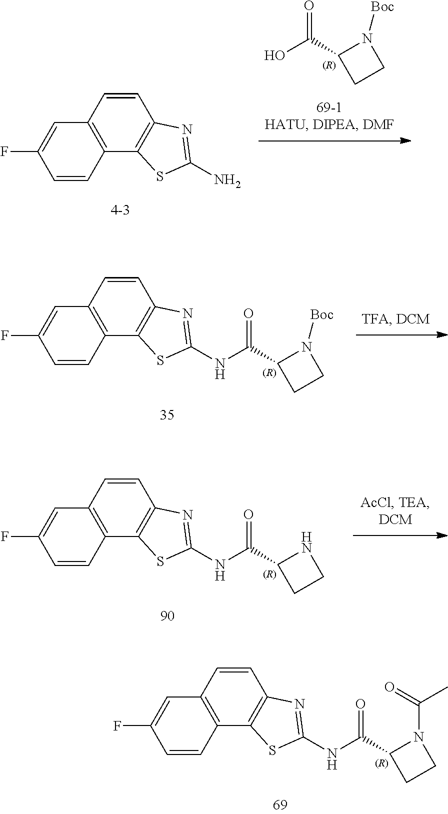

### Tert-butyl (R)-2-((7-fluoronaphtho[2,1-d]thiazol-2-yl)carbamoyl)azetidine-1-carboxylate (35)

To a stirred solution of (R)-1-(tert-butoxycarbonyl)azetidine-2-carboxylic acid (69-1, 0.22 g, 1.10 mmol) in DMF (5 mL), DIPEA (0.5 mL, 2.75 mmol) was added followed by HATU (0.52 g, 1.37 mmol) at 0° C. and the reaction mixture was stirred at RT for 15 min. To the resulting reaction mixture compound 4-3 (0.20 g, 0.91 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (30% EtOAc/hexane) to afford 35 (0.2 g, 54.5%) as a light brown solid. ¹H NMR (400 MHz, DMSO-d₆) δ 12.58-12.78 (m, 1H), 8.17 (dd, J=5.62, 8.56 Hz, 1H), 7.92-7.99 (m, 2H), 7.89 (dd, J=1.96, 10.27 Hz, 1H), 7.51-7.58 (m, 1H), 4.87 (dd, J=5.38, 8.80 Hz, 1H), 3.76-4.01 (m, 2H), 2.15-2.26 (m, 1H), 1.21-1.45 (m, 9H), 1H merged in solvent peak; LC-MS: m/z 402.0 [M+H]⁺; HPLC: 98.03%; SOR: 79.22, Solvent: Methanol, Path length: 100 mm, Concentration: 0.25 w/v %.

### (R)—N-(7-fluoronaphtho[2,1-d]thiazol-2-yl)azetidine-2-carboxamide (90)

A mixture of 35 (0.18 g, 0.44 mmol) and 30% TFA in DCM (5.5 mL) was stirred at 0° C. for 5 min and then allowed to warm to RT and stirred at RT for 2 h. The progress of the reaction was monitored by TLC. After completion of the reaction the reaction mixture was concentrated under reduced pressure. The crude compound was triturated with diethyl ether followed by pentane and dried to afford 69-3 (TFA salt, 0.18 g) as an off white solid. ¹H NMR (400 MHz, DMSO-d₆) δ 12.90-13.16 (m, 1H), 9.06-9.38 (m, 2H), 8.14-8.23 (m, 1H), 7.87-8.05 (m, 3H), 7.51-7.63 (m, 1H), 5.21-5.33 (m, 1H), 3.98-4.11 (m, 1H), 3.82-3.93 (m, 1H), 2.74-2.83 (m, 1H), 2.60-2.71 (m, 1H); LC-MS: m/z 301.9 [M+H]⁺; HPLC: 99.55%.

### (R)-1-acetyl-N-(7-fluoronaphtho[2,1-d]thiazol-2-yl)azetidine-2-carboxamide (69)

To a stirred solution of 90 (0.15 g, 0.36 mmol) in DCM (5 mL), TEA (0.11 g, 1.80 mmol) was added at RT and the reaction mixture was stirred for 10 min. The mixture was cooled to 0° C. and treated with acetyl chloride (0.06 g, 0.72 mmol). The reaction mixture was allowed to warm to RT and stirred at RT for 2 h. The progress of the reaction was monitored by TLC. After completion of the reaction, the reaction mixture was poured into cold water. The aqueous layer was extracted with DCM. The organic layers were combined and dried over anhydrous Na₂SO₄ and concentrated under reduced pressure. The crude compound was purified by prep HPLC to afford 69 (0.02 g, 82.5%) as an off white solid. ¹H NMR (400 MHz, DMSO-d₆) δ 7.99-8.17 (m, 1H), 7.74-7.97 (m, 3H), 7.42-7.58 (m, 1H), 4.89-5.03 (m, 1H), 4.09-4.22 (m, 1H), 3.73-3.87 (m, 1H), 2.09-2.34 (m, 1H), 1.68-1.86 (m, 3H), 2H merged in solvent peak; LC-MS: m/z 343.9 [M+H]⁺; HPLC: 98.87%; SOR: 118.61, Solvent: DMSO, Path length: 100 mm, Concentration: 0.25 w/v %.

### Example 38. N-(7-fluoronaphtho[2,1-d]thiazol-2-yl)-1-methylpyrrolidine-3-carboxamide (70), and N-(7-fluoronaphtho[2,1-d]thiazol-2-yl)-1-methylpyrrolidine-3-carboxamide (92) and their HCl salts (65, 48)

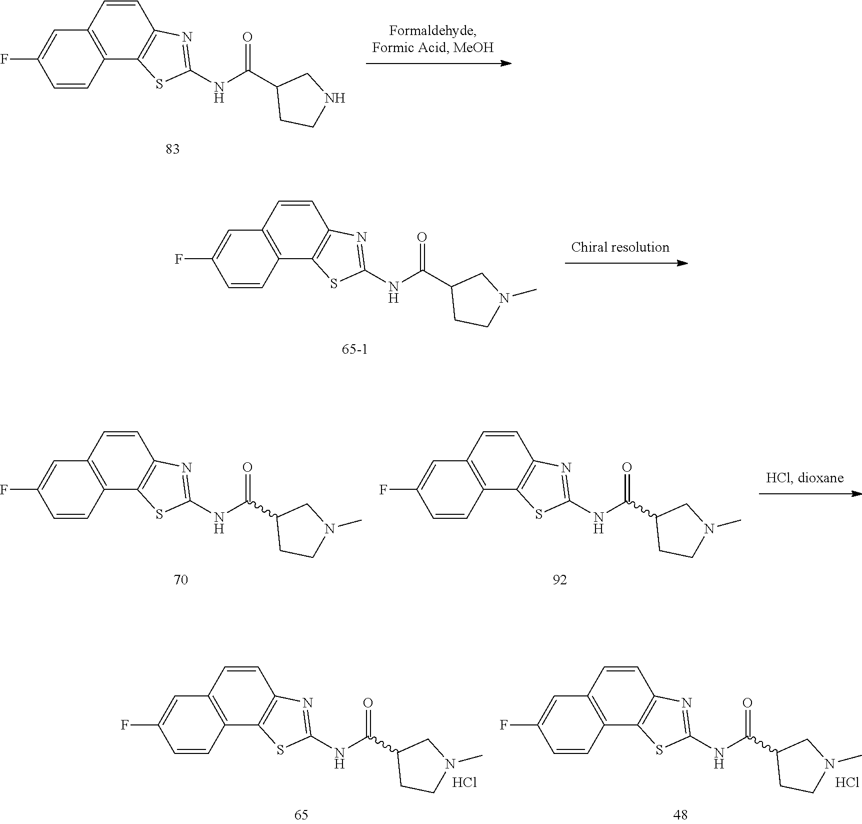

### N-(7-fluoronaphtho[2,1-d]thiazol-2-yl)-1-methylpyrrolidine-3-carboxamide (1)

To a stirred solution of 83 (0.5 g, 1.59 mmol) in MeOH (5 mL) was added formaldehyde (3 mL, 37% solution in water) and the mixture was stirred for 10 min. Formic acid (3 mL) was added and the mixture was stirred at 90° C. for 12 h. The progress of the reaction was monitored by TLC. After completion of the reaction all volatiles were evaporated and the residue was purified by silica gel column chromatography (6% MeOH/DCM) to afford compound 65-1 (0.3 g, 57.0%) as an off white solid.

Chiral prep. HPLC purification of racemic compound 65-1 afforded 70 (0.02 g, 7.0%) and 48 (0.015 g, 5.0%) both as off white solid.

Chiral Prep. HPLC Method:

Column: DIACEL CHIRALPAK-IA, 250 mm*30 mm, 5 u
Mobile Phase: A: n-Hexane+0.1% Iso-propyl-amine

B: Iso-propyl-alcohol

Flow rate: 28 mL/min

### Isocratic: 50% B

70: ¹H NMR (400 MHz, DMSO-d₆) δ 8.13 (dd, J=5.38, 8.80 Hz, 1H), 7.85-7.96 (m, 3H), 7.49-7.58 (m, 1H), 3.23-3.30 (m, 1H), 2.83 (t, J=8.80 Hz, 1H), 2.54-2.63 (m, 2H), 2.41-2.47 (m, 1H), 2.27 (s, 3H), 2.06 (q, J=7.34 Hz, 2H); LC-MS: m/z 329.9 [M+H]⁺; HPLC: 99.30%; C-HPLC: 100.00% (RT: 6.40); SOR: −36.48, Solvent: Methanol, Path length: 100 mm, Concentration: 0.25 w/v %.

48: ¹H NMR (400 MHz, DMSO-d₆) δ 8.13 (dd, J=5.38, 8.80 Hz, 1H), 7.85-7.96 (m, 3H), 7.51-7.58 (m, 1H), 3.24-3.30 (m, 1H), 2.83 (t, J=8.56 Hz, 1H), 2.53-2.62 (m, 2H), 2.43-2.49 (m, 1H), 2.27 (s, 3H), 2.06 (q, J=7.17 Hz, 2H); LC-MS: m/z 330.3 [M+H]⁺; HPLC: 99.17%; C-HPLC: 96.53% (RT: 9.65); SOR: 22.37, Solvent: Methanol, Path length: 100 mm, Concentration: 0.25 w/v %.

A 0° C. solution of 70 (20 mg, 0.06 mmol) in 1,4 dioxane (2 mL) was treated with HCl.Dioxane (2 mL, 4M). The mixture was stirred at room temperature for 2 h. All volatile were evaporated to dryness to afford 65 (10.1 mg, 45.5%) as an off white solid.

65: ¹H NMR (400 MHz, DMSO-d₆) δ 12.82 (brs, 1H), 10.31-10.94 (m, 1H), 8.11-8.18 (m, 1H), 7.87-7.99 (m, 3H), 7.56 (t, J=8.56 Hz, 1H), 4.13 (dd, J=3.42, 5.38 Hz, 1H), 3.76-3.95 (m, 2H), 3.28-3.38 (m, 1H), 3.05-3.20 (m, 1H), 2.87 (dd, J=4.89, 9.29 Hz, 3H), 2.29-2.39 (m, 1H), 2.07-2.18 (m, 1H); LC-MS: m/z 330.0 [M+H]⁺; HPLC: 95.00%.

48 was synthesized in a similar fashion as 65 to afford 48 (12 mg, 72.0%) as an off white solid.

48: ¹H NMR (400 MHz, DMSO-d₆) δ 12.82 (brs, 1H), 10.32-10.95 (m, 1H), 8.14 (brs, 1H), 7.86-7.99 (m, 3H), 7.51-7.60 (m, 1H), 3.75-3.86 (m, 1H), 3.53-3.73 (m, 2H), 3.27-3.43 (m, 1H), 3.04-3.21 (m, 1H), 2.87 (dd, J=4.65, 8.56 Hz, 3H), 2.27-2.42 (m, 1H), 2.07-2.19 (m, 1H); LC-MS: m/z 329.9 [M+H]⁺; HPLC: 98.13%.

### Example 39. (1S,3R)—N-(7-fluoronaphtho[2,1-d]thiazol-2-yl)-3-(N-methylacetamido)cyclopentane-1-carboxamide (71)

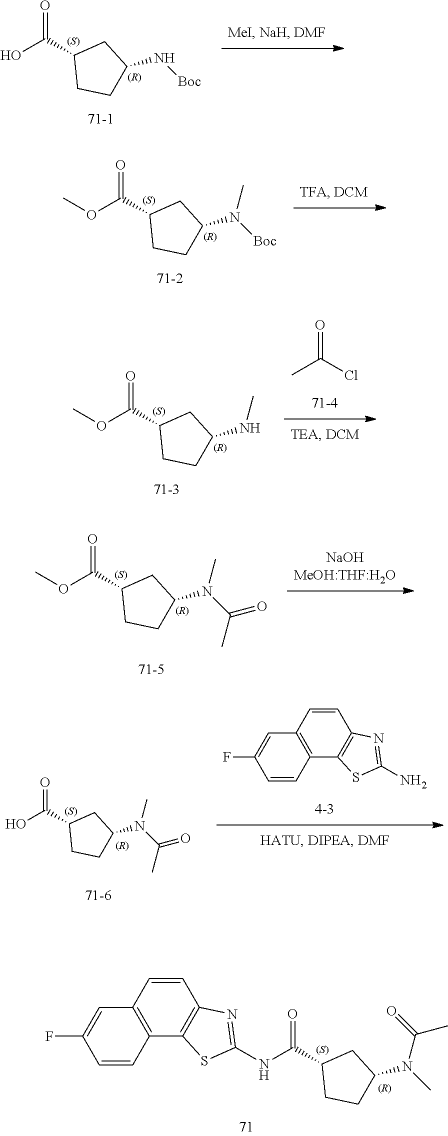

### Methyl (1S,3R)-3-((tert-butoxycarbonyl)(methyl)amino)cyclopentane-1-carboxylate (71-2)

To a stirred solution of (1S,3R)-3-((tert-butoxycarbonyl)amino)cyclopentane-1-carboxylic acid (71-1, 1.0 g, 4.37 mmol) in DMF (10 mL) at 0° C. was added NaH (60% in mineral oil, 0.35 g, 8.70 mmol) followed by Mel (3.1 g, 21.8 mmol) and the reaction mixture was stirred at RT for 3 h. The progress of the reaction was monitored by TLC. After completion of the reaction the reaction mixture was quenched with saturated NH₄Cl solution. The aqueous layer was extracted with ethyl acetate. The organic layer was washed with brine, dried over anhydrous Na₂SO₄ and concentrated under reduced pressure. The crude compound was triturated with pentane and dried well to afford compound 71-2 (1.1 g, 98.0%) as an off white solid. LC-MS: m/z 258.0 [M+H]⁺.

### Methyl (1S,3R)-3-(methylamino)cyclopentane-1-carboxylate (71-3)

A mixture of compound 71-2 (1.1 g, 4.28 mmol) and 30% TFA in DCM (15 mL) was stirred at 0° C. for 5 min followed by at RT for 3 h. The progress of the reaction was monitored by TLC. After completion of the reaction, the reaction mixture was concentrated under reduced pressure. The crude compound was triturated with diethyl ether followed by pentane and dried well to afford compound 71-3 (TFA salt, 1.0 g) as a light brown solid. LC-MS: m/z 158.0 [M+H]⁺.

### Methyl (1S,3R)-3-(N-methylacetamido)cyclopentane-1-carboxylate (71-5)

To a stirred solution of compound 71-3 (0.50 g, 3.18 mmol) in DCM (10 mL) was added TEA (1.37 mL, 9.50 mmol) at RT and the reaction mixture was stirred for 10 min. To the resulting reaction mixture acetyl chloride (0.37 g, 4.70 mmol) was added at 0° C. and the reaction mixture was stirred at RT for 2 h. The progress of the reaction was monitored by TLC. After completion of the reaction the reaction mixture was poured into cold water. The aqueous layer was extracted with DCM. The organic layers were combined and dried over anhydrous Na₂SO₄ and concentrated under reduced pressure. The crude compound was purified by silica gel column chromatography (2% MeOH/DCM) to afford compound 71-5 (0.30 g, 47.0%) as colorless thick oil. LC-MS: m/z 200.0 [M+H]⁺.

### (1S,3R)-3-(N-methylacetamido)cyclopentane-1-carboxylic acid (71-6)

To a stirred solution of compound 71-5 (0.30 g, 1.50 mmol) in MeOH/THF (6 mL) and water (1 mL) was added NaOH (0.12 g, 3.00 mmol) and the reaction mixture was stirred at RT for 2 h. The progress of the reaction was monitored by TLC. After completion of the reaction the reaction mixture was diluted with water and extracted with ethyl acetate. The aqueous layer was acidified with 2N HCl to pH 3. The aqueous layer was extracted with 10% MeOH/DCM. The organic layer was dried over anhydrous Na₂SO₄ and concentrated under reduced pressure to afford compound 71-6 (0.2 g, 72.0%) as an off white solid. LC-MS: m/z 186.0 [M+H]⁺.

### (1S,3R)—N-(7-fluoronaphtho[2,1-d]thiazol-2-yl)-3-(N-methylacetamido)cyclopentane-1-carboxamide (71)

To a stirred solution of compound 71-6 (0.19 g, 1.03 mmol) in DMF (3 mL) at 0° C. was added DIPEA (0.4 mL, 2.0 mmol) followed by HATU (0.39 g, 2.65 mmol) and the reaction mixture was stirred at RT for 15 min. To the resulting reaction mixture compound 4-3 (0.15 g, 0.68 mmol) was added and the reaction mixture was stirred at RT for 12 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (70% EtOAc/hexane) followed by prep HPLC to afford 71 (0.02 g, 5.0%) as an off white solid. ¹H NMR (400 MHz, DMSO-d₆) δ 12.55 (d, J=12.23 Hz, 1H), 8.13 (dd, J=5.87, 8.80 Hz, 1H), 7.85-7.97 (m, 3H), 7.51-7.58 (m, 1H), 4.25-4.98 (m, 1H), 3.02-3.15 (m, 1H), 2.72-2.90 (m, 3H), 2.08-2.17 (m, 1H), 1.98-2.08 (m, 3H), 1.89-1.97 (m, 2H), 1.63-1.87 (m, 3H); LC-MS: m/z 396.10 [M+H]⁺; HPLC: 99.36%; C-HPLC: 98.36% (RT: 11.46); SOR: 74.06, Solvent: DMSO, Path length: 100 mm, Concentration: 0.25 w/v %.

### Example 40. (S)-1-acetyl-N-(7-fluoronaphtho[2,1-d]thiazol-2-yl)azetidine-2-carboxamide (73) and Its Intermediates

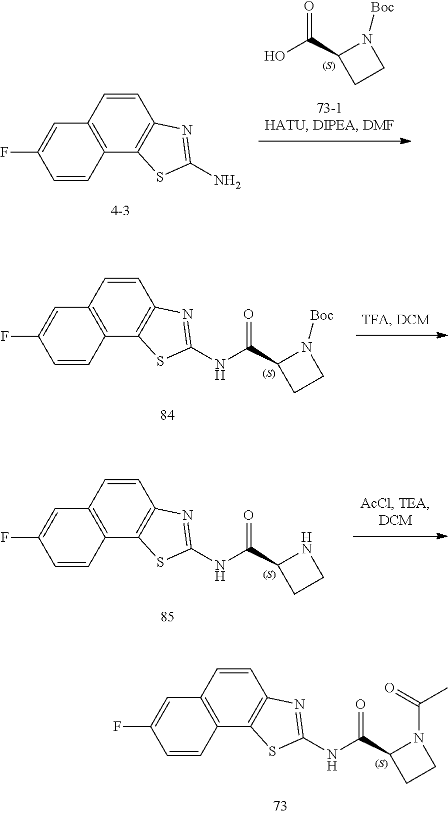

### Tert-butyl (S)-2-((7-fluoronaphtho[2,1-d]thiazol-2-yl)carbamoyl)azetidine-1-carboxylate (84)

To a stirred solution of (S)-1-(tert-butoxycarbonyl)azetidine-2-carboxylic acid (73-1, 0.22 g, 1.10 mmol) in DMF (5 mL). DIPEA (0.50 mL, 2.75 mmol) was added followed by HATU (0.52 g, 1.38 mmol) at 0° C. The reaction mixture was allowed to warm to RT and stirred at RT for 15 min. To the resulting reaction mixture compound 4-3 (0.20 g, 0.92 mmol) was added and the reaction mixture was stirred at RT for 14 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (50% EtOAc/hexane) to afford 84 (0.21 g, 57.0%) as an off white solid. ¹H NMR (400 MHz, DMSO-d₆) δ 12.59-12.79 (m, 1H), 8.17 (dd, J=5.38, 8.80 Hz, 1H), 7.86-8.00 (m, 3H), 7.51-7.59 (m, 1H), 4.87 (dd, J=5.14, 8.56 Hz, 1H), 3.91-4.05 (m, 1H), 3.75-3.90 (m, 1H), 2.13-2.27 (m, 1H), 1.19-1.46 (m, 9H), 1H merged in solvent peak; LC-MS: m/z 402.5 [M+H]⁺; HPLC: 98.16%; SOR: −81.51, Solvent: DMSO, Path length: 100 mm, Concentration: 0.25 w/v %.

### (S)—N-(7-fluoronaphtho[2,1-d]thiazol-2-yl)azetidine-2-carboxamide (85)

A mixture of 84 (0.18 g, 0.45 mmol) and 30% TFA in DCM (7 mL) was stirred at 0° C. for 5 min followed by at RT for 1 h. The progress of the reaction was monitored by TLC. After completion of the reaction, the reaction mixture was concentrated under reduced pressure. The crude compound was triturated with diethyl ether followed by pentane and dried well to afford 85 (TFA salt, 0.15 g) as an off white solid. ¹H NMR (400 MHz, DMSO-d₆) δ 13.15 (brs, 1H), 9.12-9.44 (m, 2H), 8.19 (dd, J=5.38, 8.80 Hz, 1H), 7.88-8.02 (m, 3H), 7.54-7.61 (m, 1H), 5.27 (t, J=8.07 Hz, 1H), 4.00-4.11 (m, 2H), 2.72-2.83 (m, 1H), 2.60-2.71 (m, 1H); LC-MS: m/z 302.4 [M+H]⁺; HPLC: 98.53%.

### (S)-1-acetyl-N-(7-fluoronaphtho[2,1-d]thiazol-2-yl)azetidine-2-carboxamide (73)

To a stirred solution of 85 (0.15 g, 0.50 mmol) in DCM (5 mL), TEA (0.21 mL, 1.50 mmol) was added at RT and the reaction mixture was stirred for 10 min. The reaction mixture was cooled to 0° C. and treated with acetyl chloride (0.06 g, 0.75 mmol). The reaction mixture was allowed to warm to RT and stirred at RT for 1 h. The progress of the reaction was monitored by TLC. After completion of the reaction the reaction mixture was poured into cold water. The aqueous layer was extracted with DCM. The combined organic layers were dried over anhydrous Na₂SO₄ and concentrated under reduced pressure. The crude compound was purified by prep HPLC to afford 73 (0.011 g, 6.5%) as an off white solid. ¹H NMR (400 MHz, DMSO-d6) δ 8.13 (dd, J=5.38, 8.80 Hz, 1H), 8.03 (dd, J=5.62, 9.05 Hz, 1H), 7.85-7.97 (m, 2H), 7.76-7.83 (m, 1H), 7.44-7.58 (m, 1H), 4.90-5.04 (m, 1H), 4.09-4.22 (m, 1H), 3.73-3.86 (m, 1H), 2.11-2.31 (m, 1H), 1.70-1.86 (m, 3H), 1H merged in solvent peak; LC-MS: m/z 343.9 [M+H]⁺; HPLC: 99.25%; SOR: −132.89, Solvent: DMSO, Path length: 100 mm, Concentration: 0.25 w/v %.

### Example 41. (S)—N-(7-fluoronaphtho[2,1-d]thiazol-2-yl)tetrahydrofuran-2-carboxamide (78)

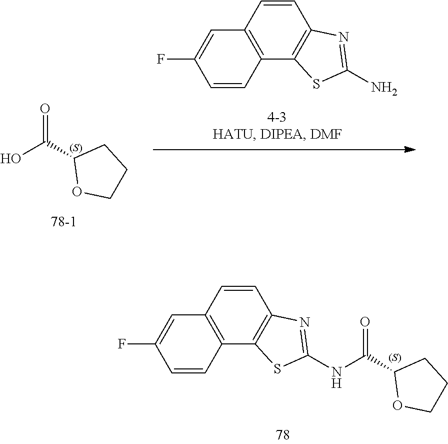

### (S)—N-(7-fluoronaphtho[2,1-d]thiazol-2-yl)tetrahydrofuran-2-carboxamide (78-1)

To a stirred solution of (S)-tetrahydrofuran-2-carboxylic acid (78-1, 0.08 g, 0.68 mmol) in DMF (3 mL) at 0° C. was added DIPEA (0.25 mL, 1.37 mmol) followed by HATU (0.26 g, 0.68 mmol). The reaction mixture was stirred at RT for 15 min. To the resulting reaction mixture was added compound 4-3 (0.10 g, 0.46 mmol) and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (30% EtOAc/hexane) to afford 78 (0.05 g, 35.0%) as an off white solid. ¹H NMR (400 MHz, DMSO-d₆) δ 12.36 (brs, 1H), 8.12-8.19 (m, 1H), 7.85-7.98 (m, 3H), 7.50-7.58 (m, 1H), 4.58-4.66 (m, 1H), 3.97-4.05 (m, 1H), 3.84 (q. J=6.68 Hz, 1H), 2.20-2.31 (m, 1H), 1.99-2.09 (m, 1H), 1.91 (dd, J=6.60, 13.45 Hz, 2H); LC-MS: m/z 317.10 [M+H]⁺; HPLC: 99.27%. SOR: −44.80, Solvent: Methanol, Path length: 100 mm, Concentration: 0.25 w/v %.

### Example 42. (1R,3S)-3-acetamido-N-(7-fluoronaphtho[2,1-d]thiazol-2-yl)cyclohexane-1-carboxamide (79)

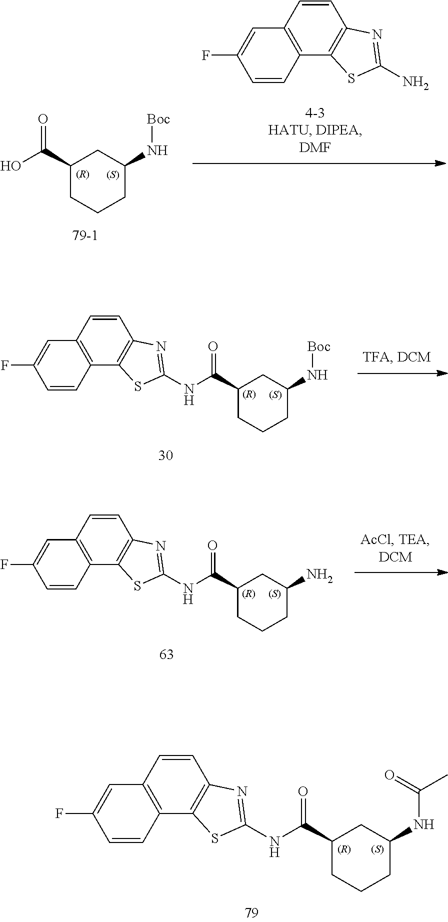

### Tert-butyl ((1S,3R)-3-((7-fluoronaphtho[2,1-d]thiazol-2-yl)carbamoyl)cyclohexyl) carbamate (30)

To a stirred solution of (1R,3S)-3-((tert-butoxycarbonyl)amino)cyclohexane-1-carboxylic acid (79-1, 0.22 g, 0.92 mmol) in DMF (5 mL) cooled to 0° C. was added DIPEA (0.5 mL, 2.75 mmol) followed by HATU (0.52 g, 1.37 mmol). The reaction mixture was stirred at RT for 15 min. To the resulting reaction mixture compound 4-3 (0.20 g, 0.92 mmol) was added and the reaction mixture was stirred at RT for 16 h. The progress of the reaction was monitored by TLC. After completion of the reaction, the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (50% EtOAc/hexane) to afford 79-30 (0.2 g, 49.0%) as an off white solid. ¹H NMR (400 MHz, DMSO-d₆) δ 12.46 (brs, 1H), 8.09-8.16 (m, 1H), 7.84-7.96 (m, 3H), 7.50-7.58 (m, 1H), 6.86 (d, J=7.83 Hz, 1H), 2.59-2.71 (m, 1H), 1.98 (d, J=12.72 Hz, 1H), 1.74-1.88 (m, 3H), 1.38 (s, 9H), 1.25-1.35 (m, 3H), 1.09-1.19 (m, 1H), 1H merged in solvent peak; LC-MS: m/z 444.10 [M+H]⁺; HPLC: 99.23%; SOR: 66.62, Solvent: DMSO, Path length: 100 mm, Concentration: 0.2 w/v %.

### (1R,3S)-3-amino-N-(7-fluoronaphtho[2,1-d]thiazol-2-yl)cyclohexane-1-carboxamide (63)

A mixture of 30 (0.2 g, 0.45 mmol) and 30% TFA in DCM (6 mL) was stirred at 0° C. for 5 min followed by at RT for 3 h. The progress of the reaction was monitored by TLC. After completion of the reaction, the reaction mixture was concentrated under reduced pressure. The crude compound was triturated with diethyl ether followed by pentane and dried to afford 63 (TFA salt, 0.14 g) as an off white solid. ¹H NMR (400 MHz, DMSO-d₆) δ 12.59 (s, 1H), 8.14 (dd, J=5.38, 8.80 Hz, 1H), 7.83-7.97 (m, 5H), 7.50-7.59 (m, 1H), 3.11 (d, J=3.42 Hz, 1H), 2.70 (t, J=11.98 Hz, 1H), 2.11 (d, J=11.74 Hz, 1H), 1.83-2.01 (m, 3H), 1.55 (q, J=11.90 Hz, 1H), 1.20-1.43 (m, 3H); LC-MS: m/z 344.20 [M+H]⁺; HPLC: 99.88%; SOR: 12.67, Solvent: DMSO, Path length: 100 mm, Concentration: 0.3 w/v %.

### (1R,3S)-3-acetamido-N-(7-fluoronaphtho[2,1-d]thiazol-2-yl)cyclohexane-1-carboxamide (79)

To a stirred solution of 63 (0.12 g, 0.35 mmol) in DCM (5 mL) was added TEA (0.10 mL, 1.04 mmol) at RT and the reaction mixture was stirred for 10 min. The resulting reaction mixture was cooled to 0° C. and treated with acetyl chloride (0.03 g, 0.42 mmol). The reaction mixture was stirred at RT for 12 h. The progress of the reaction was monitored by TLC. After completion of the reaction the reaction mixture was poured into cold water. The aqueous layer was extracted with DCM. The organic layer was dried over anhydrous Na₂SO₄ and concentrated under reduced pressure. The crude compound was purified by prep HPLC to afford 79 (0.012 g, 9.0%) as an off white solid. ¹H NMR (400 MHz, DMSO-d₆) δ 12.48 (brs, 1H), 8.10-8.16 (m, 1H), 7.79-7.96 (m, 4H), 7.49-7.58 (m, 1H), 3.60 (dd, J=3.91, 7.83 Hz, 1H), 2.63-2.74 (m, 1H), 1.98 (d, J=12.23 Hz, 1H), 1.80-1.90 (m, 3H), 1.78 (s, 3H), 1.28-1.41 (m, 3H), 1.05-1.18 (m, 1H); LC-MS: m/z 386.05 [M+H]⁺; HPLC: 99.54%; SOR: 115.04, Solvent: Methanol, Path length: 100 mm, Concentration: 0.275 w/v %.

### Example 43. (1S,3R)—N-(7-fluoronaphtho[2,1-d]thiazol-2-yl)-3-(methylamino)cyclopentane-1-carboxamide (87)

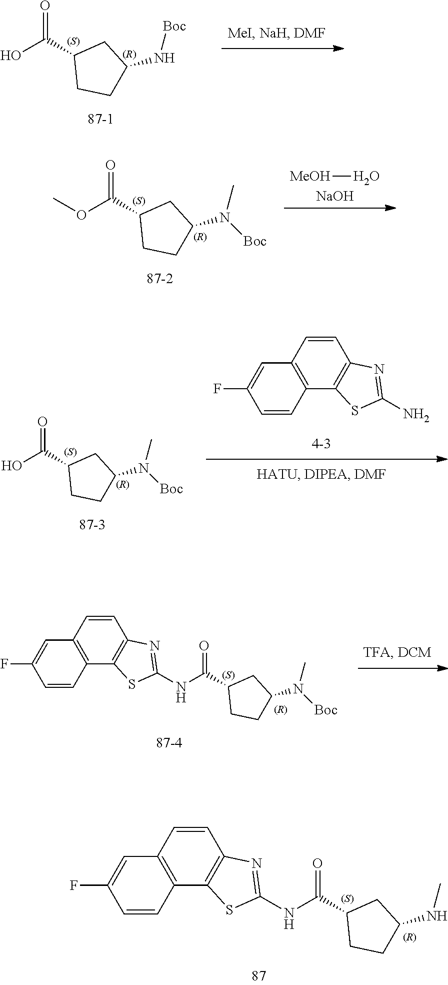

### Methyl (1S,3R)-3-((tert-butoxycarbonyl)(methyl)amino)cyclopentane-1-carboxylate (87-2)

To a stirred solution of (1S,3R)-3-((tert-butoxycarbonyl)amino)cyclopentane-1-carboxylic acid (87-1, 1.0 g, 4.37 mmol) in DMF (10 mL), was added NaH (60% in mineral oil, 0.35 g, 8.70 mmol) at 0° C. followed by Mel (3.1 g, 21.8 mmol) and the reaction mixture was stirred at RT for 3 h. The progress of the reaction was monitored by TLC. After completion of the reaction the reaction was quenched with saturated NH₄Cl solution. The aqueous layer was extracted with ethyl acetate. The organic layer was washed with brine, dried over anhydrous Na₂SO₄ and concentrated under reduced pressure. The crude compound was triturated with pentane and dried well to afford compound 87-2 (1.1 g, 98.0%) as an off white solid. LC-MS: m/z 258.0 [M+H]⁺.

### (1S,3R)-3-((tert-butoxycarbonyl)(methyl)amino)cyclopentane-1-carboxylic acid (87-3)

To a stirred solution of compound 87-2 (0.35 g, 1.36 mmol) in MeOH:H₂O (1:1, 5 mL) was added NaOH (0.11 g, 2.72 mmol) and the reaction mixture was stirred at 70° C. for 1 h. The progress of the reaction was monitored by TLC. After completion of the reaction the reaction mixture was diluted with water and extracted with ethyl acetate. The aqueous layer was acidified with 2N HCl to pH 3. The aqueous layer was extracted with 10% MeOH/DCM. The organic layer was dried over anhydrous Na₂SO₄, concentrated under reduced pressure and triturated with diethyl ether followed by pentane to afford compound 87-3 (0.3 g, 91.0%) as an off white solid. LC-MS: m/z 244.0 [M+H]⁺.

### Tert-butyl ((1R,3S)-3-((7-fluoronaphtho[2,1-d]thiazol-2-yl)carbamoyl)cyclopentyl)(methyl)carbamate (87-4)

To a stirred solution of compound 87-3 (0.25 g, 1.03 mmol) in DMF (5 mL) was added DIPEA (0.27 g, 2.06 mmol) followed by HATU (0.39 g, 1.03 mmol) at 0° C. and the reaction mixture was stirred at RT for 15 min. To the resulting reaction mixture compound 4-3 (0.15 g, 0.7 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (70% EtOAc/hexane) to afford compound 87-4 (0.07 g, 23.0%) as an off white solid. LC-MS: m/z 444.0 [M+H]⁺.

### (1S,3R)—N-(7-fluoronaphtho[2,1-d]thiazol-2-yl)-3-(methylamino)cyclopentane-1-carboxamide (87)

A mixture of compound 87-4 (0.06 g, 0.13 mmol) and 30% TFA in DCM (3.5 mL) was stirred at 0° C. for 5 min followed by RT for 12 h. The progress of the reaction was monitored by TLC. After completion of the reaction the reaction mixture was concentrated under reduced pressure. The crude compound was triturated with diethyl ether followed by pentane and dried well to afford 87 (TFA salt, 0.05 g) as an off white solid. ¹H NMR (400 MHz, DMSO-d₆) δ 12.63 (brs, 1H), 8.60 (brs, 2H), 8.13 (dd, J=5.62, 8.56 Hz, 1H), 7.84-8.00 (m, 3H), 7.49-7.61 (m, 1H), 3.06-3.21 (m, 1H), 2.60 (s, 3H), 2.32-2.41 (m, 1H), 1.83-2.15 (m, 5H), 1.71-1.82 (m, 1H); LC-MS: m/z 344.05 [M+H]⁺; HPLC: 99.09%; SOR: 5.17, Solvent: DMSO, Path length: 100 mm, Concentration: 0.25 w/v %.

### Example 44. (S)—N-(7-fluoronaphtho[2,1-d]triazol-2-yl)tetrahydrofuran-3-carboxamide (91)

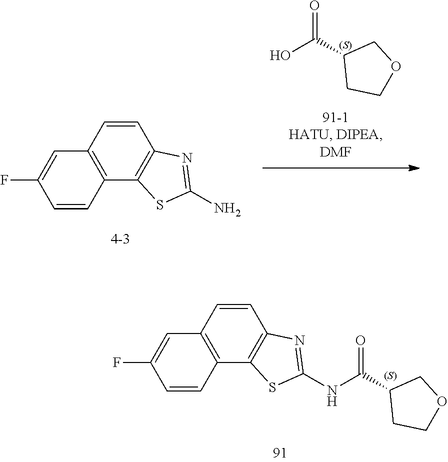

To a stirred solution of (S)-tetrahydrofuran-3-carboxylic acid (91-1, 0.10 g, 0.83 mmol) in DMF (5 ml) at 0° C. was added DIPEA (0.27 g, 2.06 mmol) followed by HATU (0.39 g, 1.03 mmol) and the reaction mixture was stirred at RT for 15 min. To the resulting reaction mixture compound 4-3 (0.15 g, 0.69 mmol) was added and the reaction mixture was stirred at RT for 16 h. The progress of the reaction was monitored by TLC. After completion of the reaction the reaction mixture was diluted with water. The aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (50% EtOAc/hexane) to afford 91 (0.045 g, 20.5%) as light brown solid. ¹H NMR (400 MHz, DMSO-d₆) δ 12.64 (s, 1H), 8.10-8.17 (m, 1H), 7.85-7.97 (m, 3H), 7.50-7.58 (m, 1H), 3.92-4.01 (m, 1H), 3.78-3.87 (m, 2H), 3.69-3.78 (m, 1H), 3.35-3.43 (m, 1H), 2.10-2.22 (m, 2H); LC-MS: m/z 317.05 [M+H]⁺; HPLC: 96.87%; C-HPLC: 95.80% (RT: 6.26); SOR: 12.03, Solvent: DMSO, Path length: 100 mm, Concentration: 0.25 w/v %.

### Example 45. Biological Assay Protocol(s)/Data

In vitro kinase assay was performed in white, 384-low volume plates in 5 μl reaction volume consisting of 30 ng CDK9/CycTI (Carna biosciences #04-110), 30 μM CDKtide (Signalchem #C06-58) and 10 μM ATP made in reaction buffer (40 mM Tris, pH 7.5; 20 mM MgCl2; 0.1 mg/ml BSA; 50 μM DTT) at 1 μM & 10 μM compound concentration for Tier1 assay and 8 to 10-point dose response curve for Tier2 assay. After 2 hours of incubation at room temperature, the amounts of remaining ATP in the kinase reaction were quantified using ADP-Glo™ Kinase Assay Kit (Promega #V9102). Briefly, 5 μl of ADP-Glo™ reagent was added to stop the kinase reaction and plate was incubated for 40 minutes at room temperature. Subsequently, 10 μl of Kinase Detection Reagent was added and luminescence was recorded after 30 minutes of incubation at room temperature.

The inhibition of kinase activity was determined relative to positive control (2% DMSO) and IC50 was calculated using GraphPad prism software (four parameter-variable slope equation).

IC50 values for the compounds used in Example 87 can be found in Table 2. As set forth in table 2 below, an IC50 value of greater than or equal to 0.001 μM and less than or equal to 0.1 μM is marked “A”; a value greater than 0.10 μM and less than or equal to 0.5 μM is marked “B”; a value greater than 0.5 μM and less than or equal to 1.0 μM is marked “C”; and a value greater than 1.0 μM and less than 20.0 μM is marked “D.”

TABLE 2

| | IC₅₀ |
|---|---|
| Patent No. | Score |
| 1 | D |
| 2 | D |
| 3 | B |
| 4 | A |
| 5 | D |
| 6 | C |
| 7 | A |
| 8 | A |
| 9 | A |
| 10 | B |
| 11 | A |
| 12 | B |
| 13-a | A |
| 13-b | B |
| 14 | A |
| 15-a | A |
| 15-b | C |
| 16 | A |
| 17 | A |
| 18-a | B |
| 18-b | D |
| 19 | A |
| 20 | D |
| 21 | A |
| 22 | B |
| 23 | A |
| 24 | D |
| 25 | C |
| 26 | A |
| 27 | A |
| 28 | B |
| 29 | D |
| 30 | B |
| 31 | B |
| 32 | D |
| 33 | B |
| 34 | B |
| 35 | D |
| 36 | A |
| 37 | A |
| 38 | B |
| 39 | A |
| 40 | D |
| 41 | A |
| 42 | B |
| 43 | B |
| 44 | A |
| 45 | A |
| 46 | C |
| 47 | C |
| 48 | A |
| 49 | D |
| 50 | D |
| 51 | A |
| 52 | A |
| 53 | D |
| 54 | A |
| 55 | A |
| 57 | A |
| 58 | B |
| 59 | C |
| 60 | A |
| 61 | A |
| 62 | D |
| 63 | B |
| 64 | C |
| 65 | B |
| 66 | D |
| 67 | D |
| 68 | C |
| 69 | A |
| 70 | A |
| 71 | A |
| 72 | A |
| 73 | D |
| 74 | C |
| 75 | A |
| 76 | A |
| 77 | D |
| 78 | B |
| 79 | A |
| 80 | D |
| 81 | C |
| 82 | B |
| 83 | A |
| 84 | D |
| 85 | B |
| 86 | A |
| 87 | A |
| 88 | A |
| 89 | D |
| 90 | C |
| 91 | B |
| 92 | A |

1. A compound of formula (I): 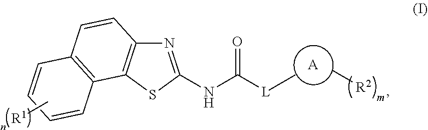
or a pharmaceutically acceptable salt thereof,
wherein
L is selected from a group consisting of a bond, an optionally substituted C₁-C₃ alkylene chain, or —C(H)═C(H)—;
Ring A is selected from the group consisting of optionally substituted C₃-C₆ carbocyclyl, optionally substituted phenyl, optionally substituted naphthyl, optionally substituted 5-10-membered heteroaryl containing 1-3 heteroatoms selected from the group consisting of N, O, and S, and optionally substituted 5-10-membered heterocyclyl containing 1-3 heteroatoms selected from the group consisting of N, O, and S;
each R¹ is independently selected from the group consisting of halogen, —CN, —OR^{a}, —NR^{a}R^{b}, —CO₂R^{b}, optionally substituted C₁-C₆ alkyl, C₁-C₃ haloalkyl, optionally substituted C₁-C₆ alkoxy, and C₁-C₃ haloalkoxy;
each R² is independently selected from the group consisting of halogen, oxo, —CN, —OH, —NR^{a}R^{b}, —NR^{a}C(O)R^{d}, —S(O)₂R^{c}, —NR^{a}S(O)₂R^{c}, —C(O)R^{d}, —C(O)OH, optionally substituted C₁-C₆alkyl, C₁-C₃ haloalkyl, optionally substituted C₁-C₆ alkoxy, C₁-C₃ haloalkoxy, optionally substituted phenyl, optionally substituted 5-6-membered heteroaryl containing 1-3 heteroatoms selected from the group consisting of N, O, and S, and optionally substituted 5-6-membered heterocyclyl containing 1-3 heteroatoms selected from the group consisting of N, O, and S;
R^{a} is hydrogen or optionally substituted C₁-C₆ alkyl;
R^{b} is hydrogen or optionally substituted C₁-C₆ alkyl;
R^{c} is optionally substituted C₁-C₆ aliphatic or optionally substituted phenyl;
R^{d} is optionally substituted C₁-C₆ alkyl, C₁-C₃ haloalkyl, optionally substituted phenyl, optionally substituted benzyl or —O(optionally substituted C₁-C₆ alkyl);
n is 0, 1, 2, or 3; and
m is 0, 1, 2, or 3.

2. The compound of claim 1, wherein the compound is of formula (I-a)

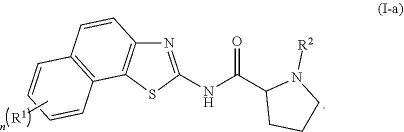

3. The compound of claim 1, wherein the compound is of formula (I-b)

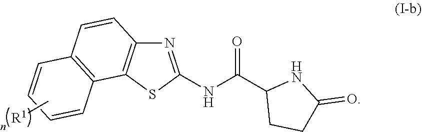

4. The compound of claim 1, wherein Ring A is selected from the group consisting of:

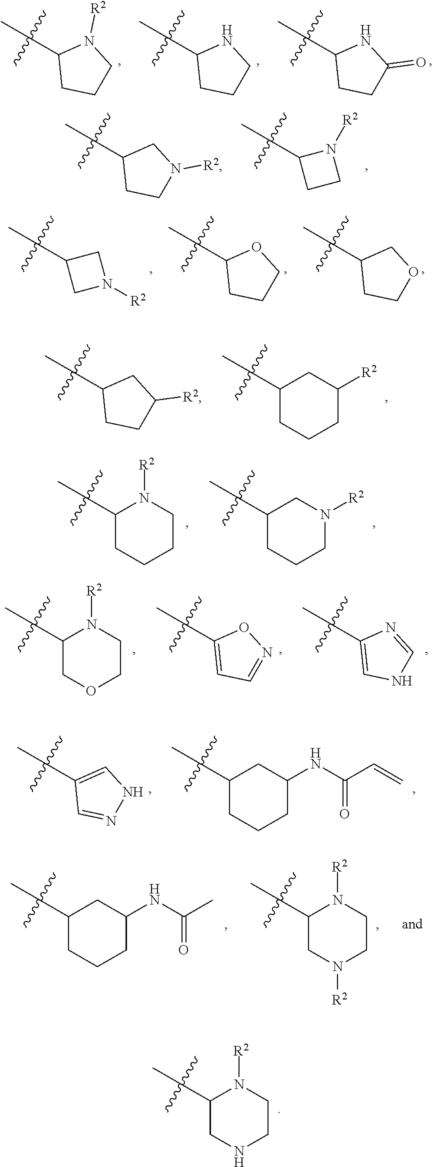

5. The compound of any of claims 1-4, wherein R² is —C(O)R^{d}.

6. The compound of any of claims 1-5, wherein R^{d} is methyl.

7. A compound selected from the group consisting of

| Compound No. | Structure |
|---|---|
| 1 | 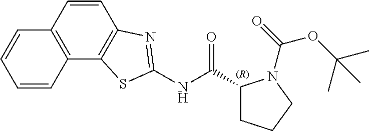 |
| 2 | 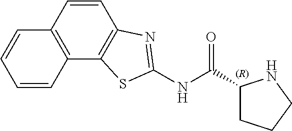 |
| 3 | 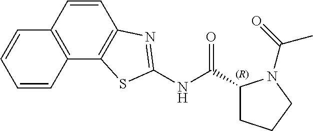 |
| 4 | 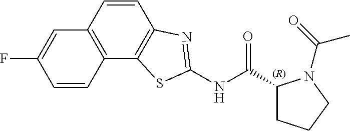 |
| 5 | 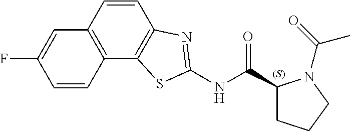 |
| 6 | 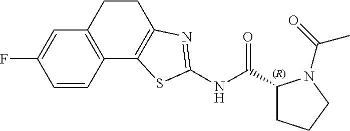 |
| 7 | 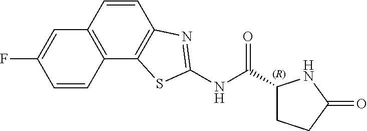 |
| 8 | 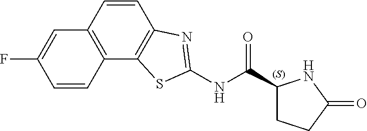 |
| 9 | 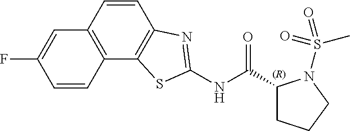 |
| 10 | 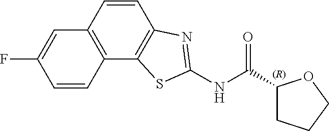 |
| 11 | 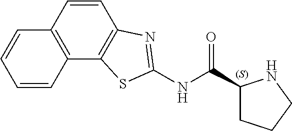 |
| 12 | 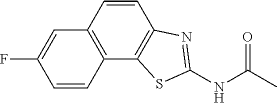 [SMILES: CC(=O)Nc1nc2ccc3cc(F)ccc3c2s1] |
| 13-a | 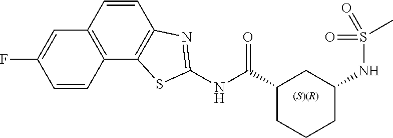 |
| 13-b | 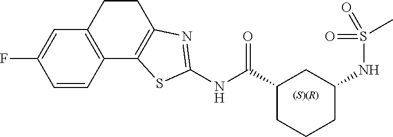 |
| 14 | 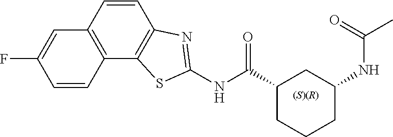 |
| 15 | 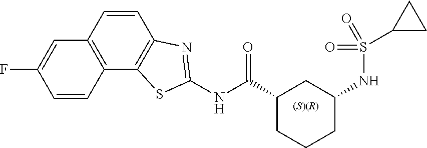 |
| 16 | 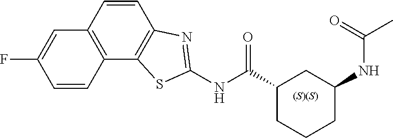 |
| 17 | 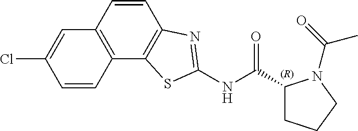 |
| 18-a* | 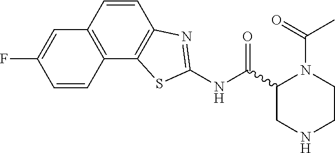 [SMILES: CC(=O)N1CCNCC1C(=O)Nc1nc2ccc3cc(F)ccc3c2s1] |
| 18-b* | 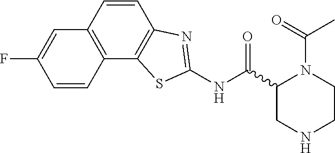 [SMILES: CC(=O)N1CCNCC1C(=O)Nc1nc2ccc3cc(F)ccc3c2s1] |
| 19 | 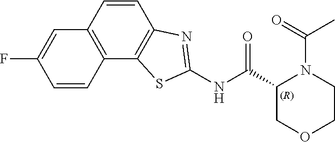 |
| 20 | 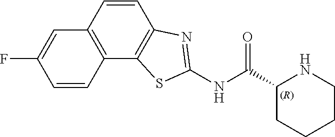 |
| 21 | 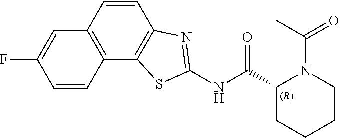 |
| 22 | 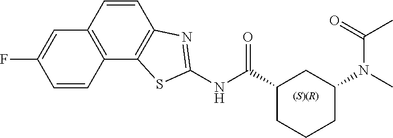 |
| 23 | 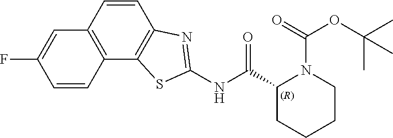 |
| 24 | 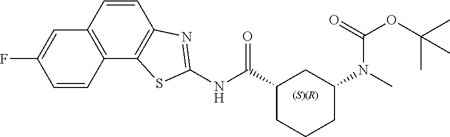 |
| 25 | 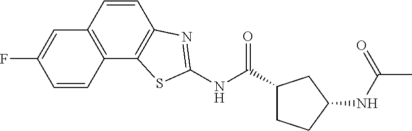 [SMILES: CC(=O)N[C@@H]1CC[C@H](C(=O)Nc2nc3ccc4cc(F)ccc4c3s2)C1] |
| 26 | 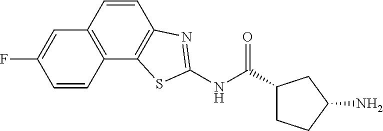 [SMILES: N[C@@H]1CC[C@H](C(=O)Nc2nc3ccc4cc(F)ccc4c3s2)C1] |
| 27 | 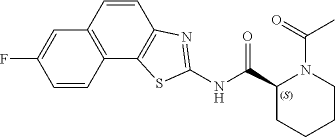 |
| 28 | 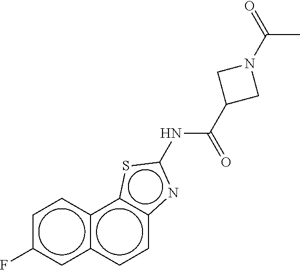 |
| 29 | 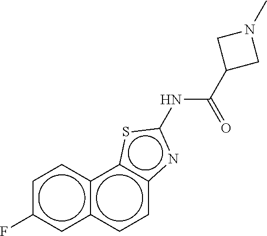 |
| 30 | 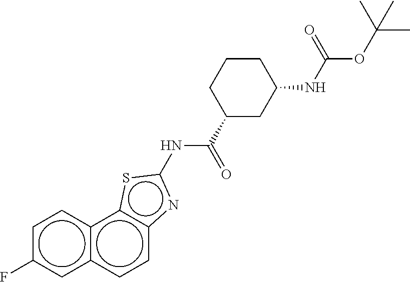 |
| 31 | 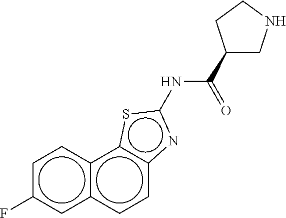 |
| 32 | 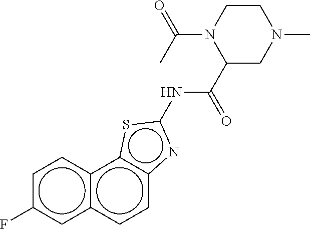 |
| 33 | 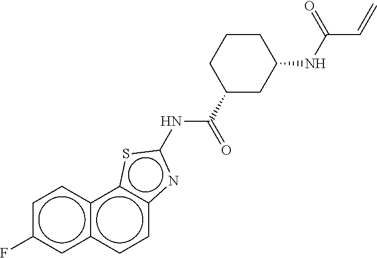 |
| 34 | 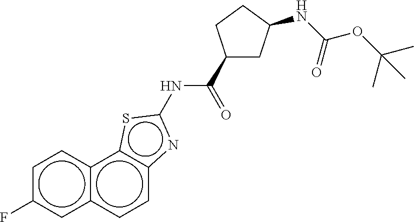 |
| 35 | 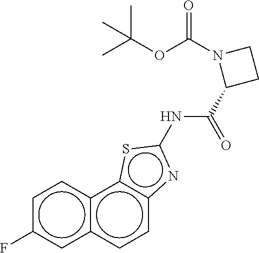 |
| 36 | 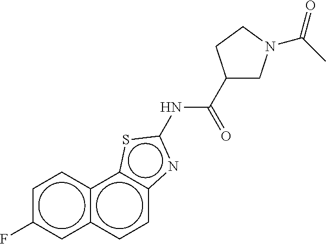 |
| 37 | 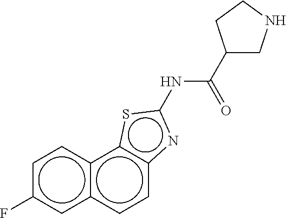 |
| 38 | 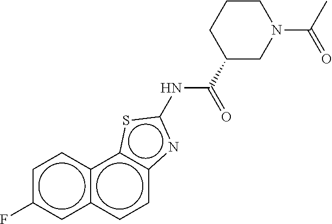 |
| 39 | 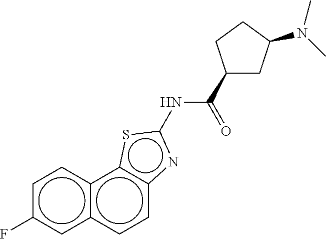 |
| 40 | 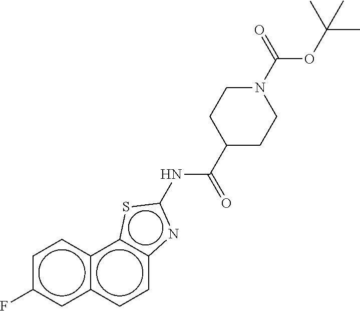 |
| 41 | 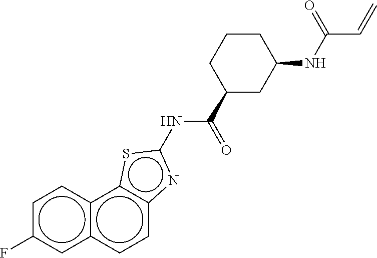 |
| 42 | 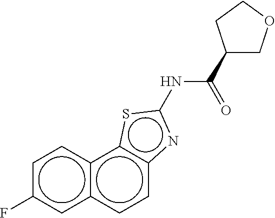 |
| 43 | 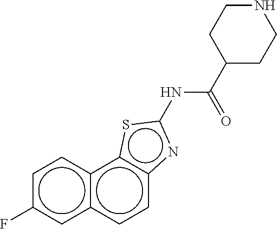 |
| 44 | 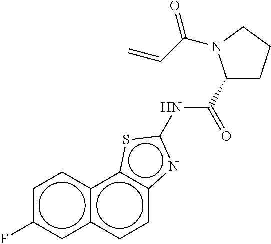 |
| 45 | 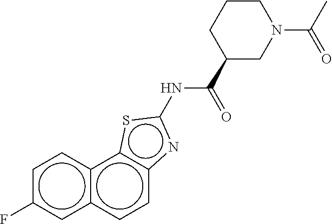 |
| 46 | 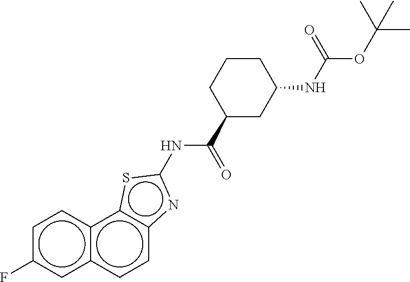 |
| 47 | 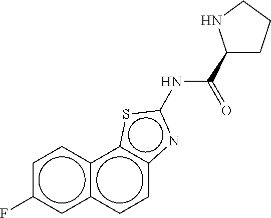 |
| 48 | 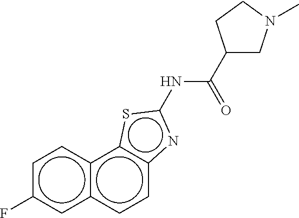 |
| 49 | 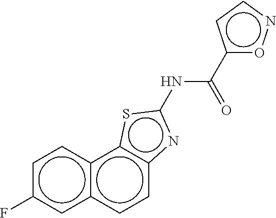 |
| 50 | 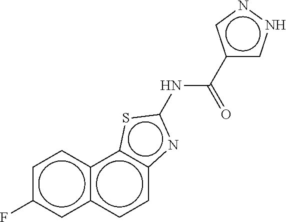 |
| 51 | 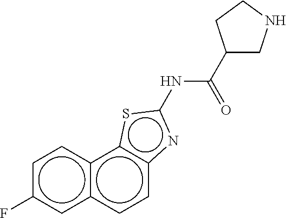 |
| 52 | 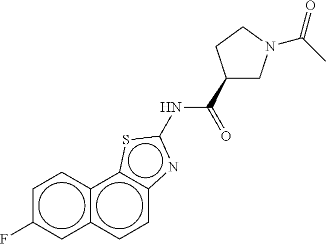 |
| 53 | 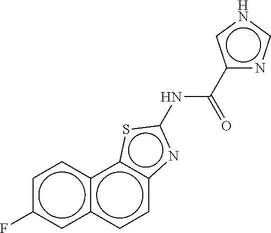 |
| 54 | 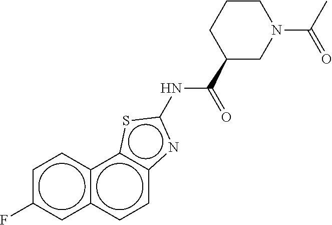 |
| 55 | 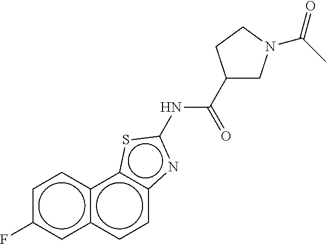 |
| 57 | 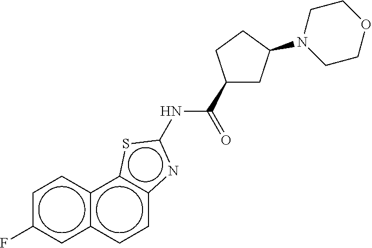 |
| 58 | 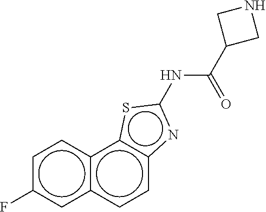 |
| 59 | 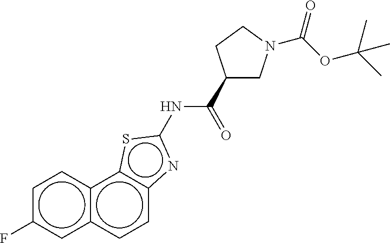 |
| 60 | 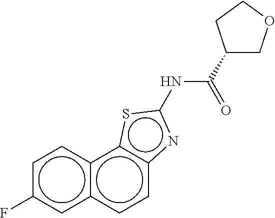 |
| 61 | 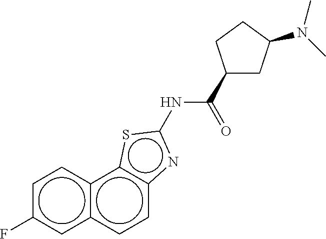 |
| 62 | 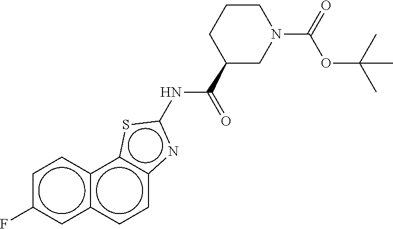 |
| 63 | 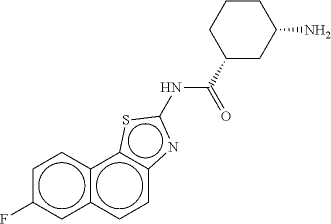 |
| 64 | 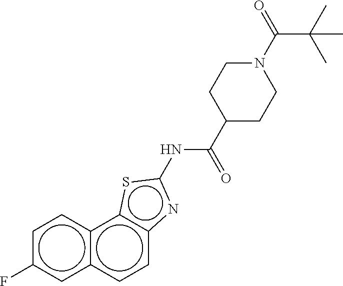 |
| 65 | 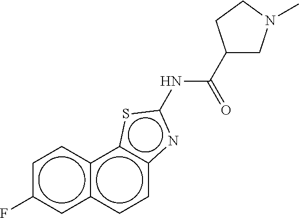 |
| 66 | 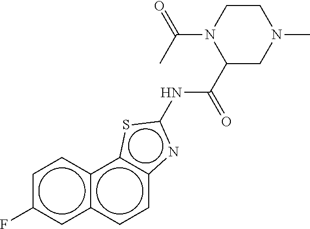 |
| 67 | 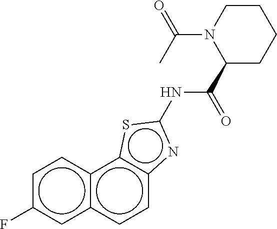 |
| 68 | 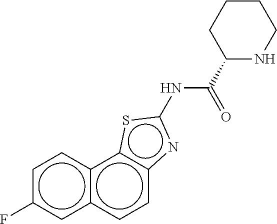 |
| 69 | 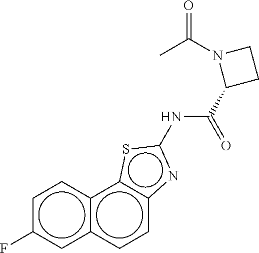 |
| 70 | 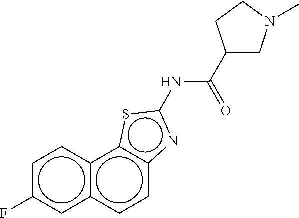 |
| 71 | 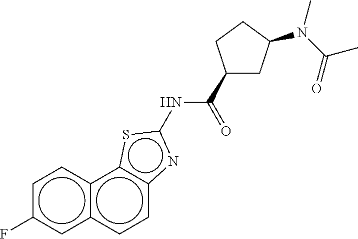 |
| 72 | 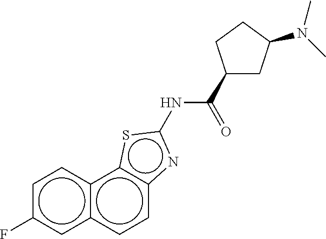 |
| 73 | 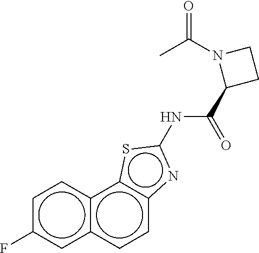 |
| 74 | 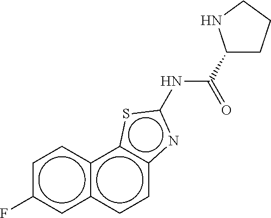 |
| 75 | 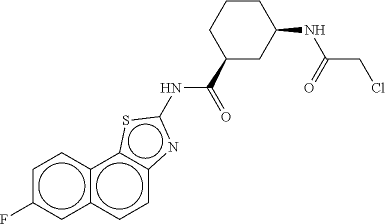 |
| 76 | 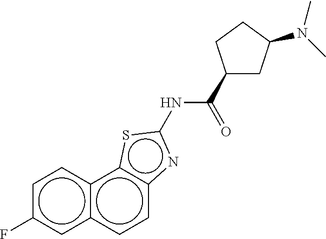 |
| 77 | 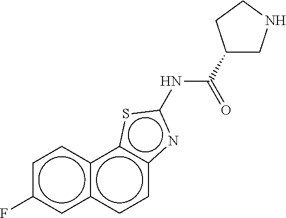 |
| 78 | 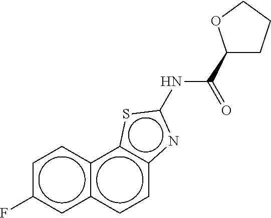 |
| 79 | 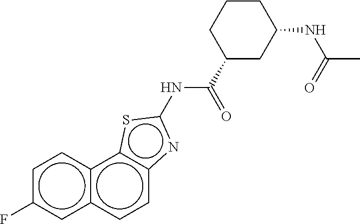 |
| 80 | 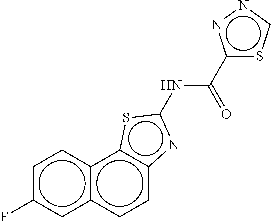 |
| 81 | 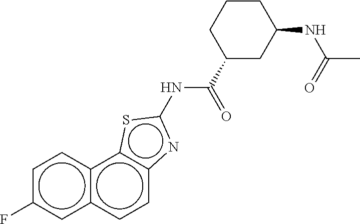 |
| 82 | 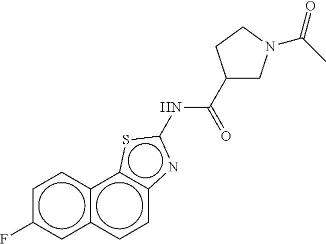 |
| 83 | 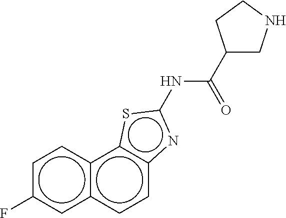 |
| 84 | 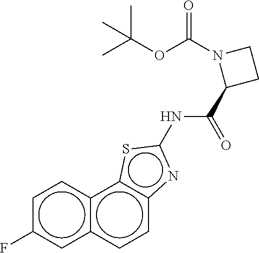 |
| 85 | 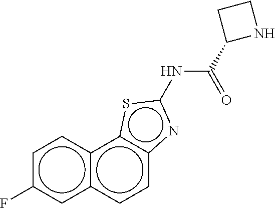 |
| 86 | 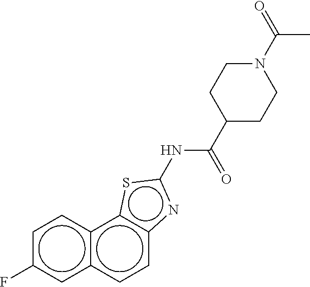 |
| 87 | 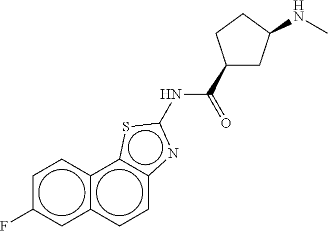 |
| 88 | 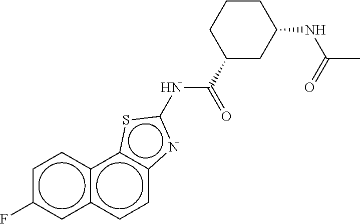 |
| 89 | 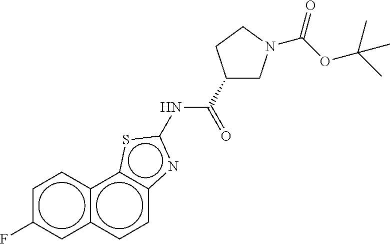 |
| 90 | 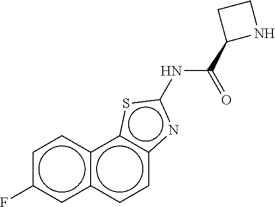 |
| 91 | 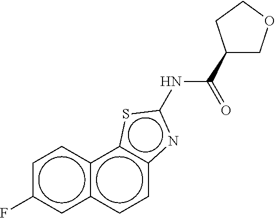 |
| 92 | 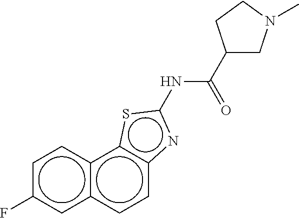 |

or a pharmaceutically acceptable salt thereof.

8. A pharmaceutical composition comprising a compound of any of claims 1-7 and a pharmaceutically acceptable carrier, adjuvant or vehicle.

9. A method for inhibiting the activity of CDK9, or a mutant thereof, in a patient or in a biological sample comprising a step of administering to said patient or contacting said biological sample with a compound of any of claims 1-7 or the pharmaceutical composition of claim 8.

10. A method of treating a disease or disorder in a patient in need thereof comprising a step of administering to said patient a compound of any of claims 1-7 or the pharmaceutical composition of claim 8.

11. The method of claim 10 wherein, the disease or disorder is cancer.

12. The method of claim 11, wherein the cancer is selected from the group consisting of non-small cell lung carcinoma, prostate carcinoma, pancreatic ductal adenocarcinoma, cervical carcinoma, melanoma comprising, glioma, acute myeloid leukemia, multiple myeloma, chronic lymphocytic leukemia, diffuse large B cell lymphoma, Burkitt's lymphoma, follicular lymphoma breast cancer, lung cancer, neuroblastoma and colon cancer.

13. The method of claim 12, wherein the cancer is glioma.

14. The method of any of claims 9-13 further comprising administration of an additional therapeutic agent.
